(19) United States
(12) Patent Application Publication
(10) Pub. No.: US 2021/0116454 A1
(43) Pub. Date: Apr. 22, 2021

(54) METHODS FOR TREATING A HEMATOLOGICAL CANCER AND THE USE OF COMPANION BIOMARKERS FOR 2-(2,6-DIOXOPIPERIDIN-3-YL)-4-((2-FLUORO-4-((3-MORPHOLINOAZETIDIN-1-YL)METHYL)BENZYL)AMINO)ISOINDOLINE-1,3-DIONE

(71) Applicant: CELGENE CORPORATION, Summit, NJ (US)

(72) Inventors: Maria Soraya Carrancio ANTON, San Diego, CA (US); Celia Fontanillo FONTANILLO, San Diego, CA (US); Shailaja KASIBHATLA, San Diego, CA (US); Antonia LOPEZ-GIRONA, San Diego, CA (US); Gang LU, San Diego, CA (US); Kai WANG, San Diego, CA (US)

(73) Assignee: CELGENE CORPORATION, Summit, NJ (US)

(21) Appl. No.: 17/075,594

(22) Filed: Oct. 20, 2020

Related U.S. Application Data
(60) Provisional application No. 62/924,044, filed on Oct. 21, 2019.

(51) Int. Cl. G01N 33/574; A61K 31/5377; A61K 45/06; A61P 35/00

(52) U.S. Cl. CPC G01N 33/57484; A61P 35/00; A61K 45/06; A61K 31/5377

(57) ABSTRACT

A method of identifying a subject having a hematological cancer who is likely to be responsive to a treatment compound, comprising administering the treatment compound to the subject having the hematological cancer; obtaining a sample from the subject; determining the level of a biomarker in the sample from the subject; and diagnosing the subject as being likely to be responsive to the treatment compound if the level of the biomarker in the sample of the subject changes as compared to a reference level of the biomarker; wherein the treatment compound is Compound 1, Compound 2, or Compound 3.

### CROSS-REFERENCE TO RELATED APPLICATIONS

This application claims the benefit of U.S. Provisional Patent Application No. 62/924,044, filed Oct. 21, 2019, which is incorporated by reference herein in its entirety.

### 1. FIELD

Provided herein are methods for identifying and diagnosing patients with hematological cancer, such as diffuse large B-cell lymphoma (DLBCL) or chronic lymphocytic leukemia/small lymphocytic lymphoma (CLL/SLL). In particular, provided herein are methods of determining the expression level of certain biomarkers for identifying patients whose hematological cancer, such as DLBCL or CLL/SLL, is likely to be responsive to treatment with a compound of Formula (I) or an enantiomer, mixture of enantiomers, tautomer, isotopolog, or pharmaceutically acceptable salt thereof. Also provided herein are methods of treating such patients with a compound of Formula (I) or an enantiomer, mixture of enantiomers, tautomer, isotopolog, or pharmaceutically acceptable salt thereof that incorporate the above methodology. Further provided here in are kits for carrying out the methods described herein.

### 2. BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and metastasis. Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Current cancer therapy may involve surgery, chemotherapy, hormonal therapy and/or radiation treatment to eradicate neoplastic cells in a patient. Recent advances in cancer therapeutics are discussed by Rajkumar et al. in Nature Reviews Clinical Oncology 11, 628-630 (2014).

NHL is the fifth most common cancer for both men and women in the United States. An estimated 385,700 patients worldwide were diagnosed with NHL in 2012 and approximately 199,700 patients died as a result of the disease. (Torre, L. A. et al. Global cancer statistics, 2012; CA Cancer J. Clin. 65, 87-108 (2015)). Diffuse large B-cell lymphoma (DLBCL) accounts for approximately one-third of non-Hodgkin's lymphoma (NHL), and is the most common form of B-cell NHL, DLBCL had an estimated 27,650 new cases in the USA in 2016, accounting for approximately 26% of all mature B-cell NHL neoplasms diagnosed. (Teras, L. R. et al. 2016 US lymphoid malignancy statistics by World Health Organization subtypes; CA Cancer J. Clin. 66, 443-459 (2016)). While some DLBCL patients are cured with traditional chemotherapy, the remainder die from the disease.

A major obstacle to the treatment of DLBCL with current therapies is the ability of certain lymphomas to acquire resistance to or be refractory to the standard front-line therapy R-CHOP (rituximab, cyclophosphamide, doxorubicin, vincristine, and prednisone) or to newer agents like venetoclax and ibrutinib. Approximately 30 to 40% of patients will develop relapsed/refractory disease that remains a major cause of morbidity and mortality due to the limited therapeutic options (Camicia et al. Mol. Cancer 14, 207 (2015)). As a result, patients with relapsed/refractory DLBCL have a poor prognosis.

Recent advances in gene expression profiling have led to the identification of at least three distinct molecular subtypes of DLBCL: a germinal center B cell-like subtype, an activated B cell-like subtype, and a primary mediastinal B-cell lymphoma subtype. The ABC-DLBCL subtype is associated with a very poor prognosis when treated with CHOP only, the majority of ABC-DLBCL patients treated with CHOP alone will succumb to their disease. The 3-year progression free survival (PFS) rate and overall survival (OS) rate for R-CHOP treated patients with ABC-DLBCL are approximately 40% and 45%, respectively, while the corresponding PFS and OS rate for R-CHOP treated patients with GCB-DLBCL are approximately 74% and 80%, respectively. These groups of patients pose a particular urgent clinical need because of a very aggressive clinical course, high chemorefractoriness and inferior overall survival when treated with R-CHOP.

In addition, disease progression in lymphoma patients has been associated with impaired immune system function. For example, T-cell exhaustion has been observed in B cell non-Hodgkin's lymphoma (NHL) patients (Yang, 2014; Yang, 2015). Exhausted T cells show reduced differentiation, proliferation and function in cytokine production. Therefore, improvement in the immune system function activation of the immune system may help treatment of a hematological cancer, such as DLBCL.

Cytotoxic chemotherapy predictably suppresses the hematopoietic system, impairing host protective mechanisms, and is a serious toxicity associated with cancer chemotherapy. The degree and duration of neutropenia determine the risk of infection (Crawford, 2004). As a result, preclinical assessment of myeloid toxicity remains crucial for development of new therapeutic options for hematological cancer patients. Neutrophils represent the first line of defense against infection as the first cellular component of the inflammatory response and a key component of innate immunity. Moreover, neutropenia blunts the inflammatory response to nascent infections, allowing bacterial multiplication and invasion. Therefore, assessment of neutropenia can help in monitoring a hematological cancer treatment, such as the treatment regimen for DLBCL or CLL/SLL.

Chronic lymphocytic leukemia (CLL) is a lymphoproliferative malignancy characterized by the progressive accumulation of morphologically mature but functionally incompetent B lymphocytes in the blood, bone marrow, and lymphoid tissues with a unique cluster of differentiation (CD) CD19+, CD5+, and CD23+ phenotype. It is the most common leukemia in North America and Europe with an incidence of 4.0 cases per 100,000 persons per year that affects mainly elderly patients with the median age at presentation of 72 years. The clinical course of CLL ranges from indolent disease with long-term survival over 12 years to aggressive disease with median survival of 2 years and is influenced by stage at presentation and certain disease-specific characteristics such as cytogenetic abnormalities. Current clinical course and prognosis reflect an evolving therapeutic landscape including emerging newer agents becoming available for the treatment of CLL. Despite the recent introduction of several highly effective agents CLL remains an incurable disease for patients who do not undergo allogeneic stem cell transplantation and therefore warrants development of alternative and additional treatment options.

The molecular pathogenesis of CLL/SLL is a complex, multi-faceted process characterized by specific genetic aberrations and represents the convergence of alterations in cell signaling pathways including the B-cell receptor and apoptotic pathways, and the influence of the tumor-immune microenvironment. The term CLL is used when the disease manifests primarily in the blood, whereas the term small lymphocytic lymphoma (SLL) is used when involvement is primarily nodal. Specifically, SLL as defined by the International Workshop on Chronic Lymphocytic Leukemia (iwCLL) criteria, is a disease in patients who would otherwise be diagnosed as CLL, but which presents with a relatively normal peripheral lymphocyte count, and which requires the presence of lymphadenopathy and/or splenomegaly. In contrast to CLL, which is often found in the blood and bone marrow, as well as other disease locations, such as lymph nodes, spleen and extranodal locations, patients with SLL have less prominent manifestations in the peripheral blood.

As evidenced by recent regulatory approvals of several new targeted agents such as ibrutinib and venetoclax, the CLL treatment landscape is evolving. However, despite the availability of these newer agents, patients continue to relapse or are refractory to treatment. Moreover, patients with poor risk cytogenetic features continue to have worse outcomes compared with patients without these characteristics. Improved and novel combination treatments for CLL will remain an important medical need. In addition, the increased use of targeted therapies has triggered the emergence of novel mutations that have been shown to confer resistance to therapy. For example, resistance to the BTK inhibitor ibrutinib has been associated with mutations either in the BTK binding site or mutations that resulted in autonomous B-cell receptor activity. Therefore, exploration of agents with novel mechanisms is important to offer treatment options with unique mechanism of actions (MOAs) for patients who may develop resistance to emerging targeted agents.

There remains a significant need for safe and effective methods of treating, preventing and managing hematological cancer, such as DLBCL or CLL/SLL, particularly for DLBCL or CLL/SLL that is refractory to standard treatments, while reducing or avoiding the toxicities and/or side effects associated with conventional therapies. The present invention satisfies this need and provides related advantages as well.

Citation or identification of any reference in this section of this application is not to be construed as an admission that the reference is prior art to the present application.

### 3. SUMMARY OF THE INVENTION

In one aspect, provided herein is a method of identifying a subject having a hematological cancer who is likely to be responsive to a treatment compound or predicting the responsiveness of a subject having or suspected of having a hematological cancer to a treatment compound, comprising:

(a) obtaining a sample from the subject;

(b) determining a biomarker level in the sample;

(c) diagnosing the subject as being likely to be responsive to the treatment compound if:
(i) the biomarker level in the sample is detectable; or
(ii) the biomarker level in the sample is an altered level relative to a reference biomarker level; and
   wherein the treatment compound is a compound of Formula (I):

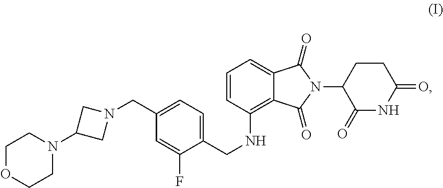 [SMILES: O=C1CCC(N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof.

In another aspect, provide herein is a method of selectively treating a hematological cancer in a subject having a hematological cancer, comprising:

(a) obtaining a sample from the subject having a hematological cancer;

(b) determining a biomarker level in the sample;

(c) diagnosing the subject as being likely to be responsive to a treatment compound if:
(i) the biomarker level in the sample is detectable; or
(ii) the biomarker level is an altered level relative to a reference level of the biomarker; and

(d) administering a therapeutically effective amount of the treatment compound to the subject diagnosed as being likely to be responsive to the treatment compound;

wherein the treatment compound is a compound of Formula (I):

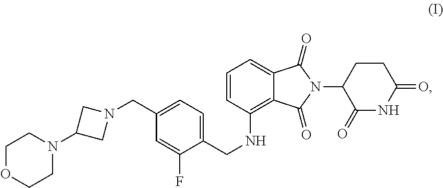 [SMILES: O=C1CCC(N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof.

In some embodiments of the methods provided herein, the biomarker is cereblon (CRBN), and the method includes diagnosing the subject as being likely to be responsive to the treatment compound if CRBN is detectable or higher than a reference level in the sample.

In other embodiments of the methods provided herein, the biomarker is Ikaros, Aiolos, ZFP91, or a combination thereof, and the method includes diagnosing the subject as being likely to be responsive to the treatment compound if the level of biomarker in the sample is lower than a reference level. In some embodiments, the biomarker is the combination of Ikaros and Aiolos, and the method includes diagnosing the subject as being likely to be responsive to the treatment compound if the level of both Ikaros and Aiolos are lower than their respective reference levels.

In another aspect, provided herein is a method of identifying a subject having a hematological cancer who is likely to be responsive to a treatment compound or predicting the responsiveness of a subject having or suspected of having a hematological cancer to a treatment compound, comprising:

(a) obtaining a sample from the subject;

(b) administering the treatment compound to the sample;

(c) determining a biomarker level in the sample; and

(d) diagnosing the subject as being likely to be responsive to the treatment compound if the biomarker level in the sample is an altered level relative to a reference biomarker level;

wherein the treatment compound is a compound of Formula (I):

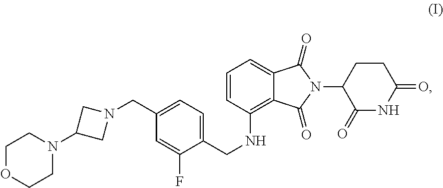 [SMILES: O=C1CCC(N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof.

In yet another aspect, provided herein is a method of identifying a subject having a hematological cancer who is likely to be responsive to a treatment compound or predicting the responsiveness of a subject having or suspected of having a hematological cancer to a treatment compound, comprising:

(a) administering a treatment compound to a subject;

(b) obtaining a sample from the subject;

(c) determining a biomarker level in the sample; and

(d) diagnosing the subject as being likely to be responsive to the treatment compound if the biomarker level in the sample is an altered level relative to a reference biomarker level;

wherein the treatment compound is a compound of Formula (I):

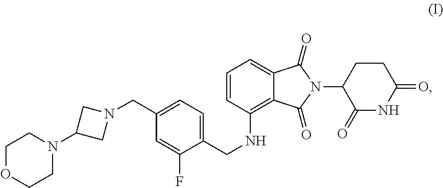 [SMILES: O=C1CCC(N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof.

In another aspect, provided herein is a method of monitoring the efficacy of a treatment compound in treating a hematological cancer in a subject, comprising:

(a) administering a treatment compound to a subject;

(b) obtaining a sample from the subject;

(c) determining a biomarker level in the sample; and

(d) comparing the biomarker level in the sample with a reference biomarker level, wherein an altered biomarker level is indicative of the efficacy of the treatment compound in treating a hematological cancer in the subject;

wherein the treatment compound is a compound of Formula (I):

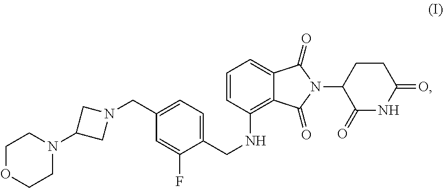 [SMILES: O=C1CCC(N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof.

In yet another aspect, provided herein is a method of adjusting a dosage amount or frequency for treating a subject having a hematological cancer with a treatment compound comprising:

(a) administering a dosage of a treatment compound to a subject;

(b) obtaining one of more samples from the subject at different time points; and

(c) monitoring a biomarker level in the one or more samples, and

(d) adjusting the dosage for subsequent administration of the treatment compound to the subject based upon an altered level of the biomarker in a reference sample,

wherein the treatment compound is a compound of Formula (I):

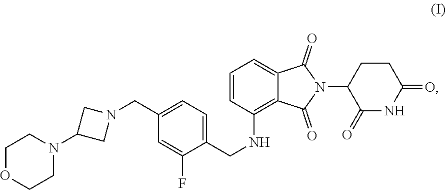 [SMILES: O=C1CCC(N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof.

In some embodiments, the methods provided herein further comprise administering a therapeutically effective amount of the treatment compound to the subject diagnosed as being likely to be responsive to the treatment compound.

In certain embodiments, the altered level of the biomarker in the sample is higher than the reference level of the biomarker. In other embodiments, the altered level of the biomarker in the sample is lower than the reference level of the biomarker.

In some embodiments, an increased biomarker level relative to the reference biomarker level is indicative of the efficacy of the treatment compound in treating the hematological cancer in the subject. In other embodiments, a decreased biomarker level relative to the reference biomarker level is indicative of the efficacy of the treatment compound in treating the hematological cancer in the subject.

In some embodiments of any one of the methods provided herein, the reference biomarker level is the biomarker level in a reference sample obtained from the subject prior to administering the treatment compound to the subject, and wherein the reference sample is from the same source as the sample. In other embodiments, the reference biomarker level is the biomarker level in a reference sample obtained from a healthy subject not having the hematological cancer, and wherein the reference sample is from the same source as the sample. In certain embodiments, the reference biomarker level is a pre-determined biomarker level.

In certain embodiments of the methods provided herein, the biomarker comprises a marker of apoptosis, and the alteration of the biomarker level is indicative of the induction of apoptosis. In specific embodiments, the biomarker is selected from cleaved caspase 3, cleaved caspase 7, cleaved poly (ADP-ribose) polymerase (PARP), BCL2, survivin, phosphatidylserine (PS) and DNA, Bcl-2-like protein 11 (BIM), tumor necrosis factor (TNF), interleukin-10 (IL-10), or interleukin-27 (IL27), or a combination thereof. In certain embodiments, the biomarker that includes a marker of apoptosis is selected from the group consisting of Annexin-V, 7-amino-actinomycin D (7-AAD), and Deep Red Anthraquinone 7 (DRAQ7), or a combination thereof. In some embodiments, the biomarker in the sample is higher than the reference level of the biomarker. In other embodiments, the biomarker in the sample is lower than the reference level of the biomarker.

In some embodiments of the methods provided herein, the biomarker is selected from IL-8, IL-1a, sPGE2, sTNFα, sIgG, sIL-17A, sIL-17F, sIL-2, sIL-6, collagen-I and -III, PAI-1, CD69, or sIL-10, or a combination thereof. In some embodiments, the biomarker in the sample is higher than the reference level of the biomarker. In other embodiments, the biomarker in the sample is lower than the reference level of the biomarker.

In certain embodiments of the methods provided herein, the biomarker is associated with interferon signaling. In specific embodiments, the biomarker associated with interfere signaling includes interleukin-6 signal transducer (IL6ST), interferon-induced transmembrane protein 3 (IFITM3), interferon alpha-inducible protein 6 (IFI6), 2′-5′-oligoadenylate synthase 3 (OAS3), interferon α (IFNα), interferon β (IFN β), or a combination thereof. In some embodiments, the biomarker in the sample is higher than the reference level of the biomarker. In other embodiments, the biomarker in the sample is lower than the reference level of the biomarker.

In some embodiments, the biomarker is associated with cytokine/chemokine signaling. In specific embodiments, the biomarker associated with cytokine/chemokine signaling includes interleukin-23 subunit alpha (IL23A), C—C motif chemokine 1 (CCL1), or a combination thereof. In some embodiments, the biomarker in the sample is higher than the reference level of the biomarker. In other embodiments, the biomarker in the sample is lower than the reference level of the biomarker.

In certain embodiments, the biomarker is associated with cell adhesion. In specific embodiments, the biomarker associated with cell adhesion includes E-selectin (SELE), P-selectin glycoprotein ligand 1 (SELPLG), thromboxane A2 (TXA2), or a combination thereof. In some embodiments, the biomarker in the sample is higher than the reference level of the biomarker. In other embodiments, the biomarker in the sample is lower than the reference level of the biomarker.

In some embodiments, the biomarker is associated with cell-cell junction. In specific embodiments, the biomarker associated with cell-cell junction includes claudin 7 (CLDN7), claudin 12 (CLDN12), or a combination thereof. In some embodiments, the biomarker in the sample is higher than the reference level of the biomarker. In other embodiments, the biomarker in the sample is lower than the reference level of the biomarker.

In certain embodiments, the biomarker is a G-protein coupled receptor. In specific embodiments, the G-protein coupled receptor includes free fatty acid receptor 2 (FFAR2). In some embodiments, the biomarker in the sample is higher than the reference level of the biomarker. In other embodiments, the biomarker in the sample is lower than the reference level of the biomarker.

In some embodiments of the methods provided herein, the biomarker is associated with extracellular matrix. In specific embodiments, the biomarker associated with the extracellular matrix comprises CD209, SERPINA, SERPINB7, or a combination thereof. In some embodiments, the biomarker in the sample is higher than the reference level of the biomarker. In other embodiments, the biomarker in the sample is lower than the reference level of the biomarker.

In certain embodiments, the biomarker is associated with cell cycle. In some embodiments, the biomarker in the sample is higher than the reference level of the biomarker. In other embodiments, the biomarker in the sample is lower than the reference level of the biomarker.

In some embodiments, the biomarker is associated with transcription. In some embodiments, the biomarker in the sample is higher than the reference level of the biomarker. In other embodiments, the biomarker in the sample is lower than the reference level of the biomarker.

In some embodiments of the methods provided herein, the biomarker includes one or more proteins selected from the group consisting of Aiolos (IKZF3), Ikaros (IKZF1), E3 ubiquitin-protein ligase ZFP91 (ZFP91), Protein C-ets-1 (ETS1), Max-binding protein MNT (MNT), myocyte-specific enhancer factor 2B (MEF2B), snRNA-activating protein complex subunit 1 (SNAPC1), lysine-specific demethylase 4B (KDM4B), transcription factor AP-4 (TFAP4), nucleolar transcription factor 1 (UBTF), bromo adjacent homology domain-containing 1 protein (BAHD1), methyl-CpG-binding domain protein 4 (MBD4), chromobox protein homolog 2 (CBX2), tumor protein 63 (TP63), transducin-like enhancer protein 3 (TLE3), forkhead box protein P1 (FOXP1), zinc finger and BTB domain-containing protein 11 (ZBTB11), interferon regulatory factor 4 (IRF4), mediator of RNA polymerase II transcription subunit 26 (MED26), cyclic AMP-dependent transcription factor ATF-7 (ATF7), zinc finger protein 644 (ZNF644), lysine-specific demethylase 5B (KDM5B), upstream stimulatory factor 2 (USF2), transcription factor 25 (TCF25), lysine-specific demethylase 4A (KDM4A), lethal (3) malignant brain tumor-like protein 2 (L3MBTL2), nRNA-activating protein complex subunit 4 (SNAPC4), lysine-specific demethylase 5 (KDM5), transcription factor COE1 (EBF1), forkhead box protein J2 (FOXJ2), nuclear factor of activated T-cells, cytoplasmic 1 (NFATC1), mRNA decay activator protein ZFP36 (ZFP36), hepatoma-derived growth factor (HDGF), ETS-related transcription factor Elf-1 (ELF1), promyelocytic leukemia protein (PML), Myb-related protein B MYBL2, mothers against decapentaplegic homolog 2 (SMAD2), chromodomain-helicase-DNA-binding protein 2 (CHD2), signal transducer and activator of transcription 1 (STAT1), Paired box protein Pax-5 (PAX5), Signal transducer and activator of transcription 2 (STAT2), pygopus homolog 2 (PYGO2), interferon regulatory factor 9 (IRF9), polycomb group RING finger protein 2 (PCGF2), and cyclic AMP-dependent transcription factor ATF-3 (ATF3). In some embodiments, the biomarker in the sample is higher than the reference level of the biomarker. In other embodiments, the biomarker in the sample is lower than the reference level of the biomarker.

In some embodiments of the methods provided herein, the biomarker includes one or more genes selected from the group consisting of Interleukin-23 subunit alpha (IL23A), C—C motif chemokine 2 (CCL2), and SLIT-ROBO Rho GTPase-activating protein 1 (SRGAP1). In some embodiments, the biomarker in the sample is higher than the reference level of the biomarker. In other embodiments, the biomarker in the sample is lower than the reference level of the biomarker.

In certain embodiments, the biomarker comprises a CRBN-associated protein or a transcriptional target of a CRBN-associated protein. In specific embodiments, the CRBN-associated protein includes IKAROS, AIOLOS, or ZFP91. In other specific embodiments, the transcriptional target of a CRBN-associated protein includes BCL6, c-MYC, or IRF4. In yet further embodiments, the transcriptional target of a CRBN-associated protein includes an interferon inducible gene. In specific embodiments, the interferon inducible gene includes interferon regulatory 7 (IRF7), interferon induced protein with tetratricopeptide repeats 3 (IFIT3), DEAD box protein 58 (DDX58), or a combination thereof. In some embodiments, the biomarker in the sample is higher than the reference level of the biomarker. In other embodiments, the biomarker in the sample is lower than the reference level of the biomarker.

In some embodiments of the methods provided herein, the biomarker is selected from the group consisting of cyclin dependent kinase inhibitor 1 (p21). In some embodiments, the biomarker in the sample is higher than the reference level of the biomarker. In other embodiments, the biomarker in the sample is lower than the reference level of the biomarker.

In certain embodiments of the methods provided herein, the biomarker includes a marker of T-cell activation. In specific embodiments, the marker of T-cell activation includes a T-cell activation associated cytokine. In some embodiments, the T-cell activation associated cytokine includes interleukin 2 (IL-2). In some embodiments, the biomarker in the sample is higher than the reference level of the biomarker. In other embodiments, the biomarker in the sample is lower than the reference level of the biomarker.

In some embodiments, the biomarker comprises PD1 and LAG3. In some embodiments, the biomarker in the sample is higher than the reference level of the biomarker. In other embodiments, the biomarker in the sample is lower than the reference level of the biomarker.

In some embodiments of the methods provided herein, the biomarker includes an effector cytokine or effector chemokine. In specific embodiments, the effector cytokine or effector chemokine includes granulocyte-macrophage colony-stimulating factor (GM-CSF), tumor necrosis factor alpha (TNFα), interferon gamma (IFNγ), or a combination thereof. In some embodiments, the biomarker in the sample is higher than the reference level of the biomarker. In other embodiments, the biomarker in the sample is lower than the reference level of the biomarker.

In certain embodiments, the biomarker is expressed in a white blood cell. In specific embodiments, the white blood cell includes a lymphoid cell. In yet more specific embodiments, the lymphoid cell includes a T-cell.

In another aspect, provided herein is a method of treating a hematological cancer, comprising:

(a) obtaining a first sample from a subject having a hematological cancer;

(b) determining a biomarker level in the first sample;

(c) administering a therapeutically effective amount of a treatment compound to the subject;

(d) obtaining at least one additional sample from the subject after the treatment; and

(e) determining the biomarker level in the at least one additional sample; and

if the biomarker level in the at least one additional sample is at or near the biomarker level of the first sample, then administering another therapeutically effective amount of the treatment compound to the subject,
wherein the treatment compound is a compound of Formula (I):

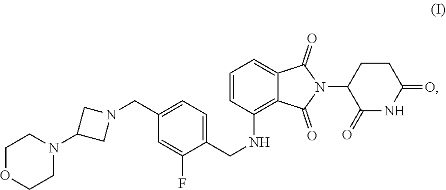 [SMILES: O=C1CCC(N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof.

In certain embodiments of the method of treating a hematological cancer, the biomarker comprises Ikaros. In specific embodiments, the Ikaros biomarker is expressed in a white blood cell. In even more specific embodiments, the white blood cell includes a myeloid cell. In some embodiments, the myeloid cell includes a neutrophil. In specific embodiments, the biomarker includes neutrophils having a phenotype of CD11b⁺, CD34⁻, and CD33⁻.

In some embodiments of any one of the methods provided herein, the compound of Formula (I) includes (S)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof. In other embodiments of any of the methods provided herein, the compound of Formula (I) includes (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof. In some embodiments of any of the methods provided herein, the compound of Formula (I) includes a mixture of (S)-2-(2,6-Dioxopiperidin-3-yl)-4-((2-fluoro-4-((3-morpholinoazetidin-1-yl)methyl)benzyl)amino)isoindoline-1,3-dione, and (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof.

In further embodiments of any of the methods provided herein, the method further includes administering a therapeutically effective amount of a second active agent or a support care therapy. In certain embodiments, the second active agent includes an HDAC inhibitor (e.g., panobinostat, romidepsin, vorinostat, or citarinostat), a BCL2 inhibitor (e.g., venetoclax), a BTK inhibitor (e.g., ibrutinib or acalabrutinib), an mTOR inhibitor (e.g., everolimus), a PI3K inhibitor (e.g., idelalisib), a PKCβ inhibitor (e.g., enzastaurin), a SYK inhibitor (e.g., fostamatinib), a JAK2 inhibitor (e.g., fedratinib, pacritinib, ruxolitinib, baricitinib, gandotinib, lestaurtinib, or momelotinib), an Aurora A kinase inhibitor (e.g., alisertib), an EZH2 inhibitor (e.g., tazemetostat, GSK126, CPI-1205, 3-deazaneplanocin A, EPZ005687, EI1, UNC1999, or sinefungin), a BET inhibitor (e.g., birabresib or 4-[2-(cyclopropylmethoxy)-5-(methanesulfonyl)phenyl]-2-methylisoquinolin-1(2H)-one), a hypomethylating agent (e.g., 5-azacytidine or decitabine), a chemotherapy (e.g., bendamustine, doxorubicin, etoposide, methotrexate, cytarabine, vincristine, ifosfamide, melphalan, oxaliplatin, or dexamethasone), an anti-CD20 monoclonal antibody (e.g., rituximab, or obinutuzumab), or an epigenetic compound, or a combination thereof. In some embodiments, the second active agent comprises rituximab. In certain embodiments, the second active agent comprises obinutuzumab.

In certain embodiments of any one of the methods provided herein, the hematological cancer affects the hematopoietic or lymphoid tissues. In some embodiments, the hematological cancer includes non-Hodgkin's lymphoma. In specific embodiments, the non-Hodgkin's lymphoma includes diffuse large B-cell lymphoma (DLBCL). In yet more specific embodiments, the DLBCL is relapsed, refractory, or resistant to conventional therapy. In other embodiments, the hematological cancer comprises chronic lymphocytic leukemia (CLL) or small lymphocytic lymphoma (SLL). In certain embodiments, the CLL/SLL is relapsed or refractory CLL/SLL.

In certain embodiments of any one of the methods provided herein, the sample includes a hematological cancer cell.

In some embodiments of any one of the methods provided herein, determining the biomarker level includes determining the protein level of the biomarker.

In some embodiments of any one of the methods provided herein, determining the biomarker level includes determining the mRNA level of the biomarker.

In certain embodiments of any one of the methods provided herein, determining the biomarker level includes determining the cDNA level of the biomarker.

### 4. BRIEF DESCRIPTION OF THE FIGURES

FIG. 1A-FIG. 1C illustrate that DLBCL cell lines expressed DLBCL related proteins Myc, BCL2, and BCL6 (FIG. 1A); as well as CRL4^{CRBN} E3 ubiquitin ligase and its substrates Aiolos, Ikaros, and ZFP91 (FIG. 1B). Quantification of CRBN expression levels were normalized to CRBN levels in DF15 cells (FIG. 1C). Beta-Tubulin was used as a loading control.

FIG. 2 illustrates that Compound 1 was active in DLBCL cell lines with acquired resistance to doxorubicin. Viability for the parental (square) and their associated Doxo-R (circle) cell lines in ABC (top panel) and GCB (bottom panel) cell lines, as well as apoptosis induction curves were generated after exposure to serially diluted Compound 1 for 5 days.

FIG. 3 illustrates representative immunoblots showing expression profiles of cereblon and relevant cereblon substrates Aiolos, Ikaros, and ZFP91 and c-Myc, IRF4, BCL2, MCL1, and BCL6 proteins in acquired-doxorubicin resistance cell lines and in the corresponding matching parental cells.

FIG. 4A and FIG. 4B illustrate that Compound 1 was selectively antiproliferative in (FIG. 4A) endothelial cells, T- and B-lymphocytes, and (FIG. 4B) coronary smooth muscle cells, and fibroblasts. The BioMAP Diversity PLUS Panel was assessed after treatment with Compound 1. The X-axis lists the quantitative protein-based biomarker readouts measured in each system. The Y-axis represents a log-transformed ratio of the biomarker readouts for the drug-treated sample (n=1) over vehicle controls (n≥6). The grey region around the Y-axis represents the 95% significance envelope generated from historical vehicle controls. Biomarker activities were annotated when 2 or more consecutive concentrations change in the same direction relative to vehicle controls, are outside of the significance envelope, and have at least one concentration with an effect size >20% (log 10 ratio >0.1). Antiproliferative effects are indicated by a thick grey arrow.

FIG. 5A-FIG. 5D illustrate that Compound 1 activity was dependent on CRBN expression. (FIG. 5A) shows that the protein expression of Aiolos, Ikaros, ZFP91, IRF4, and c-Myc were decreased, and the apoptotic proteins (cleaved caspase 7, cleaved caspase 3, and cleaved PARP), as well as the interferon stimulated gene IFIT3 were induced in a time and concentration dependent manner in SU-DHL2 cells expressing CRBN; (FIG. 5B) shows the quantification of the decreased levels of Aiolos, Ikaros, and ZFP91, as well as increased levels of cleaved caspase 3, and cleaved caspase 7, and cleaved poly (ADP-ribose) polymerase (PARP) that were first normalized to β-tubulin, then further normalized to levels of each protein at the 0-hour time point after treatment with DMSO (circle), 0.001 nM (square), 0.01 nM (upward triangle), or 0.1 nM (downward triangle) of Compound 1; (FIG. 5C) shows that no effect was observed in SU-DHL CRBN knockout cells; (FIG. 5D) shows that apoptosis increased over time using a fluorescent caspase-3 reagent in SU-DHL2^{CRBNWT} cells (top panel) after treatment with 0.01 nM (circle), 0.1 nM (square), 1.0 nM (upward triangle), 10 nM (downward triangle), 100 nM (diamond), or 1000 nM (open circle) of Compound 1, but not in SU-DHL2^{CRBN-/-} cells (bottom panel). WT=wild-type.

FIG. 6A-FIG. 6D illustrate that Compound 1 decreased expression of CRBN substrate proteins, and induced expression of apoptotic and interferon stimulated genes in DLBCL cell lines. (FIG. 6A and FIG. 6C) show that the protein expression of Aiolos, Ikaros, ZFP91, IRF4, and c-Myc were decreased, and the apoptotic proteins (cleaved caspase 7, cleaved caspase 3, and cleaved PARP), as well as the interferon stimulated genes, IRF7, DDX58, and IFIT3 were induced in a time and concentration dependent manner in TMD8 and Karpas-422 cells expressing CRBN, respectively. (FIG. 6B) shows that decreased expression of Aiolos, Ikaros, ZFP91, BCL6, IRF4, c-Myc, the anti-apoptotic proteins BIM, and survivin, and induction of the pro-apoptotic proteins (cleaved caspase 7), as well as the interferon stimulated genes IRF7, DDX58, and IFIT3 were observed in TMD8 cells after 24 hours of treatment with Compound 1. (FIG. 6D) shows that decreased expression of Aiolos, Ikaros, ZFP91, and BCL6 at 24 hours (left panel), followed by induction of p21, IRF7, IFIT3, DDX58 at 48 hours (middle panel), with an induction of pro-apoptotic proteins cleaved caspase 3, cleaved caspase 7, and cleaved PARP, and a decrease in anti-apoptotic proteins survivin and BCL2, as well as MYC were observed at 72 hours (right panel) of treatment with Compound 1 in Karpas-422 cells.

FIG. 7A and FIG. 7B illustrate that the cell fitness of DLBCL cell lines were dependent on individual cereblon substrates. (FIG. 7A) shows a schematic illustrating the design of the flow cytometry-based cellular competition assay to assess for relative changes in cell fitness upon knock-out of a gene of interest (FIG. 7B) shows the relative cell fitness of sgNT-1 (circle and solid line), sgNT-2 (circle and dashed line), sgNC-1 (diamond and solid line), sgIKZF1-1 (upward triangle and solid line), sgIKZF1-2 (upward triangle and dashed line), sgIKZF3-1 (square and solid line), sgIKZF3-2 (square and dashed line), sgZFP91-1 (downward triangle and solid line), sgZFP91-3 (downward triangle and dashed line), and sgETF1-1 (“X” and solid line) cells for the RFP+/GFP+ ratios in KARPAS-422-Cas9, U-2932-Cas9, RIVA-Cas9, SU-DHL-16-Cas9, HT-Cas9, and SU-DHL-4-Cas9 cell lines. Cells were normalized to the RFP+/GFP+ ratio of sgNT-1 cells at each timepoint. The knock-out of ETF1 (sgETF1-1) serves as a positive control for robust decrease in the RFP+/GFP+ ratio. Error bars represent standard error of the mean of 3 independent experiments.

FIG. 8A and FIG. 8B illustrate that loss of Ikaros, Aiolos, and ZFP91 sensitized DLBCL cells to Compound 1 as measured by flow cytometry-based cellular competition assay to assess for relative changes in cell fitness upon knock-out of a gene of interest (FIG. 8A) KARPAS-422-Cas9, and (FIG. 8B) SU-DHL-4-Cas9 cells. DMSO=dimethyl sulfoxide; GFP=green fluorescent protein; RFP=red fluorescent protein. Gene knock-out is indicated above each set of RFP+/GFP+ ratio. Error bars represent standard error of the mean of 3 independent experiments

FIG. 9 illustrates that treatment of DLBCL cells with Compound 1 was comparable to genetic knockout of CRBN substrates Ikaros, Aiolos, and ZFP91 as measured by immunoblot KARPAS-422-Cas9 (top left), U-2932-Cas9 (top right), and SU-DHL-4-Cas9 (bottom left). DMSO=dimethyl sulfoxide.

FIG. 10A and FIG. 10B illustrate that dual knock-out of Ikaros and Aiolos had a greater inhibition on cell fitness compared to single Ikaros or Aiolos knock-out in DLBCL cell lines. FIG. 10A shows a schematic illustrating the design of the flow cytometry-based cellular competition assay to assess relative changes in cell fitness upon knock-out of the gene(s) of interest FIG. 10B shows the relative cell fitness of sgNT-1+sgNT-2 (filled circle), sgIKZF1-1+sgNT-1 (upward triangle and solid line), sgIKZF1-1+sgNT-2 (upward triangle and dashed line), sgIKZF3-1+sgNT-1 (solid square and solid line), sgIKZF3-1+sgNT-2 (solid square and dashed line), sgIKZF1-1+sgIKZF3-1 (empty circle with solid line), and sgIKZF1-2+sgIKZF3-2 (empty circle with dashed line) cells normalized to their respective RFP+/GFP+ Day 0 ratio in KARPAS-422-Cas9, U-2932-Cas9, RIVA-Cas9, SU-DHL-16-Cas9, HT-Cas9, and SU-DHL-4-Cas9 cell lines. Error bars represent standard error of the mean of 3 independent experiments.

FIG. 11 illustrates that ectopic expression of degradation-resistant mutants of Ikaros (IKZF1-G151A) (upward triangle), Aiolos (IKZF3-G152A) (square), and ZFP91 (ZFP91-G405A) (downward triangle) provided protection from Compound 1 in KARPAS-422, RIVA, HT, and SU-DHL-4 cell lines as measured by luciferase.

FIG. 12 illustrates that Compound 1 induced the degradation of Ikaros in Peripheral Blood Mononuclear Cells (PBMCs) from four healthy donors (HD1-4). Percentage of Ikaros positive cells normalized to DMSO control after continuous exposure to Compound 1 for 3, 4, or 7 days in four donors. Ikaros was measured by flow cytometry. Data represent mean of percentage of cells positive for Ikaros. Error bars represent standard error of the mean (SEM). N=4 donors in triplicate.

FIG. 13 illustrates that Compound 1 increased the absolute levels of interleukin-2 (IL2) secretion after healthy donor PBMCs were exposed to Compound 1 for 3 (circle), 4 (square), and 7 (triangle) days. The supernatant was diluted 1:10 and IL-2 secretion was measured by MSD technology. Data points represent mean of n=3 replicates. Error bars represent standard error of the mean (SEM). DMSO=dimethyl sulfoxide; HD=healthy donor; IL-2=interleukin-2; mL=milliliter; nM=nanomolar; pg=picogram.

FIG. 14 illustrates that Compound 1 increased the fold change of interleukin-2 (IL2) secretion after healthy donor PBMCs were exposed to Compound 1 for 3 (circle), 4 (square), and 7 (triangle) days, relative to DMSO. The supernatant was diluted 1:10 and IL-2 secretion was measured by MSD technology. Data points represent mean of n=3 replicates. Error bars represent standard error of the mean (SEM). DMSO=dimethyl sulfoxide; HD=healthy donor; IL-2=interleukin-2; mL=milliliter; nM=nanomolar; pg=picogram.

FIG. 15A-FIG. 15D illustrate that Compound 1 re-stimulated T-cells in a staphylococcal enterotoxin B exhaustion assay. FIG. 15A and FIG. 15C show schematic diagrams of the SEB-induced T-cell exhaustion assay. Briefly, PBMCs were treated with 100 ng/mL SEB for 72 hours and the T-cell exhaustion phenotype was assessed by FACS analysis for PD-1 and LAG3 expression. FIG. 15B and FIG. 15D show the expression levels of PD-1 and LAG3 in control and SEB treated cells. FACS=fluorescence activated cell sorting; PBMCs=peripheral blood mononuclear cells; SEB=staphylococcal enterotoxin B; sups=supernatants.

FIG. 16 illustrates that Compounds 2 and 3 decreased the expression of Ikaros, Aiolos, and ZFP91 in SU-DHL-2 cells after treatment for 1, 2, or 6 hours with vehicle control (0.1% DMSO), Compound 1 (1, 10, 100 nM), as measured by immunoblot.

FIG. 17 illustrates that Compound 1 (circle and solid line) and Compound 2 (triangle and dashed line) degraded Ikaros in a concentration and time dependent manner in DF-15 cells expressing Enhanced ProLabel (ePL)-Aiolos, ePL-Ikaros, or ePL-ZFP91 after exposure for 45 min, 60 min, 90 min, or 3 hours.

FIG. 18A and FIG. 18B illustrate that Compound 1 did not affect the viability of neutrophil precursor (CD34+) cells, as measured by Annexin V and 7-actinomycin D (7-AAD) after exposure to DMSO (closed circle), 0.1 nM (square), 1 nM (upward triangle), 10 nM (downward triangle), 100 nM (diamond), or 1000 nM (empty circle) of Compound for 14 days (FIG. 18A) or 5 days starting on days 9 (FIG. 18B).

FIG. 19A and FIG. 19B illustrate that the percentage of mature (Stage IV) cells rebounded after CD34⁺ cells derived from healthy donor bone marrow were initially exposed to Compound 1 for 14 days (FIG. 19A) or for 5 days starting on day 9 (FIG. 19B), ex vivo, followed by washout and reincubation for 7 more days in the absence of Compound 1. Data represent the percentage of Stage IV cells defined as CD34⁻/CD33⁻/CD11b⁺.

FIG. 20A and FIG. 20B illustrate that mature (Stage IV) cells recovered after CD34⁺ cells derived from healthy donor bone marrow were exposed to 0.1 nM (square), 1 nM (upward triangle), or 10 nM (downward triangle) of Compound 1 for 14 days (FIG. 20A) or 5 days starting at day 9 of culture (FIG. 20B). DMSO (circle) served as a control. Data represent the CD34⁺ cells derived from healthy donor bone marrow percentage Stage IV cells defined as CD34⁻/CD33⁻/CD11b⁺. The thick black line at represents 50% Stage IV cells in the DMSO control.

FIG. 21 illustrates that Ikaros is initially inhibited after exposure to Compound 1 for fourteen days, and then recovers after Compound 1 washout Percentage of Ikaros inhibition compared to DMSO control upon continuous exposure to Compound 1 for 14 days with one-week washout after treatment of cells from Donors No. 1, No. 2, and No. 3. Ikaros was measured by flow cytometry every two or three days. DMSO=dimethyl sulfoxide; nM=nanomolar.

FIG. 22 illustrates that the percentage of Ikaros inhibition is initially inhibited after continuous exposure to Compound 1 for 5 days starting on Day 9, and then begins to recover after one-week washout following treatment of cells from one donor. Ikaros was measured by flow cytometry every two or three days. DMSO=dimethyl sulfoxide; nM=nanomolar.

FIG. 23 illustrates that Ikaros protein inhibition and recovery correlated with the percentage of stage IV population during ex vivo myeloid differentiation of CD34⁺ bone marrow-derived cells exposed to 0.1 nM (downward empty triangle), 1.0 nM (square), 10 nM (upward triangle), 100 nM (diamond), or 1000 nM (downward filled triangle) of Compound 1 for fourteen days with one week washout after treatment Graph presents the percentage of Stage IV cells defined as CD34⁻/CD33⁻/CD11b⁺ (solid lines) versus the percentage of Ikaros protein inhibition (dashed lines) compared to DMSO (circle) control cultures treated for 14 days. Both parameters were measured by flow cytometry every two or three days. Data represented is from 3 donors.

FIG. 24 illustrates a causal-mechanistic flow network model to infer Compound 1 effects in DLBCL models. Proteomic effects were measured at 6 and 18 hours, and transcriptional effects were measured at 12, 24, and 48 hours, and the results were integrated to identify pathways modulated by Compound 1 treatment.

FIG. 25 illustrates exemplary pathways and genes involved in Compound 1 treatment responses, such as genes associated with interferon signalling (e.g., IL6ST, IFITM3, IFI6, OAS3, interferon α/β signaling), cytokine/chemokine signaling (e.g., IL23A, CCL1), apoptosis (e.g., IL27, TNF, IL10, caspase), cell adhesion (e.g., SELE, SELPLG, TXA2PA), cell-cell junction (e.g., CLDN7, CLDN12), G-protein coupled receptors (e.g., FFAR2), extracellular matrix (e.g., CD209, SERPINA, SERPINB7), cell cycle and transcription.

FIG. 26A-FIG. 26C illustrates exemplary gene responses upon treatment with Compound 1 after 12, 24, or 48 hours. Expression of IL23A (FIG. 26A), CCL2 (FIG. 26B), and SRGAP1 (FIG. 26C) were all increased after treatment with Compound 1 in sensitive cell lines, relative to intermediate or resistant cell lines.

FIG. 27 illustrates protein expression levels of a panel of genes that are differentially expressed after treatment with Compound 1 for 6 or 18 hours.

### 5. DETAILED DESCRIPTION OF THE INVENTION

The methods provided herein are based, in part, on the discovery that a changed level, e.g., an increased level and/or a decreased level, or a detection of certain molecules (e.g., mRNAs, cDNAs, or proteins) in a biological sample can be used to identify a subject having a hematological cancer, such as diffuse large B-cell lymphoma (DLBCL) or CLL/SLL, who is likely to be responsive to a treatment compound, predict responsiveness of a subject having or suspected to have a hematological cancer, such as DLBCL or CLL/SLL, to a treatment compound, or monitor the efficacy of a treatment compound in treating a hematological cancer in a subject, where the compound is, for example, Compound 1, or a tautomer, isotopolog, or pharmaceutically acceptable salt thereof; Compound 2, or a tautomer, isotopolog, or pharmaceutically acceptable salt thereof; or Compound 3, or an enantiomer, a mixture of enantiomers, tautomer, isotopolog or pharmaceutically acceptable salt thereof.

### 5.1 Definitions

As used herein, the term “a cancer” includes, but is not limited to, solid cancer and blood borne cancer. The terms “cancer” and “cancerous” refer to or describe the physiological condition in mammals that is typically characterized by unregulated cell growth. A cancer can be a cancer of the hematopoietic and lymphoid tissue. A hematological cancer refers to a cancer that affects the blood, bone marrow, lymph, and lymphatic system.

As used herein, the term “diffuse large B-cell lymphoma (DLBCL)” refers to a neoplasm of medium or large B lymphoid cells whose nuclei are the same size as, or larger than, those of normal macrophages, or more than twice the size of those of normal lymphocytes, with a diffuse growth pattern. DLBCL is a type of Non-Hodgkin lymphoma (NHL) with at least three known subtypes: germinal center B cell type (GCB), activated B cell type (ABC), and primary mediastinal B-cell lymphoma (PMBL). DLBCL cells frequently have a concurrent rearrangement of MYC and/or BCL2 and/or BCL6. For example, in some variations, DLBCL can involve chromosomal alterations of the BCL-6 gene at the 3q27 locus, which is critical for germinal center formation, as well as additional rearrangements affecting BCL6. In addition, DLBCL can have gene rearrangements corresponding to, for example, MYC, BCL2, or BCL6 into an immunoglobulin (IG) heavy chain locus, such as a t(8;14)(q24;q32) and/or t(14;18)(q32;q21.3). The translocation of MYC, BCL6, or BCL2 to an IG locus usually leads to high levels of mRNA and protein due to the active transcription driven by a constitutively active IG promoter. Accordingly, DLBCL cells often have high levels of MYC, BCL6, or BCL2 protein.

As used herein, “subject” or “patient” is an animal, typically a mammal, including a human, such as a human patient. The term “healthy subject,” as used herein, is intended to mean an individual that does not have a hematological cancer, such as DLBCL or CLL/SLL. An exemplary “healthy subject” has no pre-existing medical conditions. However, it is understood that a “healthy subject” can have medical conditions that are unrelated to a hematological cancer, such as for example diabetes, cardiovascular disease, or any other disease or disorder that does not affect the diagnosis, treatment, biomarker level, and/or pharmacodynamics of therapy for a hematological cancer, such as DLBCL or CLL/SLL therapy.

As used herein, the terms “likely” generally refer to an increase in the probability of an event. The term “likely” when used in reference to the responsiveness of a patient generally contemplates an increased probability that the patient will be responsive to a treatment compound. The term “likely” when used in reference to a response to a treatment compound generally contemplates an increased probability that the compound will decrease the rate of disease progression or a hematological cancer cell growth. The term “likely” when used in reference to a response to a treatment compound can also generally mean the increase of indicators, such as mRNA or protein expression, that may evidence an increase in the response to a treatment compound.

As used herein, the term “responsive” or “responsiveness” when used in reference to a treatment refers to the degree of effectiveness of the treatment in lessening or decreasing the symptoms of a disease, e.g. DLBCL or CLL/SLL, being treated. For example, the term “increased responsiveness” when used in reference to a treatment of a cell or a subject refers to an increase in the effectiveness in lessening or decreasing the symptoms of the disease when measured using any methods known in the art. In certain embodiments, the increase in the effectiveness is at least about 5%, at least about 10%, at least about 20%, at least about 30%, at least about 40%, or at least about 50%, or more. However, it is also understood that responsiveness can also halt disease progression, and does not necessarily require lessening or decreasing the symptoms of the disease.

As used herein and unless otherwise indicated, the term “relapsed” refers to a disorder, disease, or condition that responded to treatment (e.g., achieved a complete response) then had progression. The treatment can include one or more lines of therapy. In one embodiment, the disorder, disease or condition has been previously treated with one or more lines of therapy. In another embodiment, the disorder, disease or condition has been previously treated with one, two, three or four lines of therapy. In one embodiment, the disorder, disease, or condition is a hematological cancer, for example DLBCL or CLL/SLL.

As used herein and unless otherwise indicated, the term “refractory” refers to a disorder, disease, or condition that has not responded to prior treatment that can include one or more lines of therapy. In one embodiment, the disorder, disease, or condition has been previously treated one, two, three or four lines of therapy. In one embodiment, the disorder, disease, or condition has been previously treated with two or more lines of treatment, and has less than a complete response (CR) to most recent systemic therapy containing regimen. In one embodiment, the disorder, disease, or condition is a hematological cancer, for example DLBCL or CLL/SLL.

In one embodiment, “relapsed or refractory” CLL/SLL may refer to CLL/SLL that has been previously treated with one or more lines of therapy. In one embodiment, the relapsed or refractory CLL/SLL is CLL/SLL that has been previously treated with one, two, three or four lines of therapy. In one embodiment, the relapsed or refractory CLL/SLL is CLL/SLL that has been previously treated with two or more lines of therapy. In one embodiment, the relapsed or refractory CLL/SLL is CLL/SLL that has been previously treated with a Bruton's tyrosine kinase (BTK) inhibitor. In one embodiment, the relapsed or refractory CLL/SLL is relapsed or refractory to a BTK inhibitor. In one embodiment, the BTK inhibitor is ibrutinib. In one embodiment, the BTK inhibitor is acalabrutinib. In one embodiment, the BTK inhibitor is zanubrutinib. In one embodiment, the BTK inhibitor is tirabrutinib.

As used herein, the term “treatment compound” refers to a compound of Formula (I), and includes Compound 1, or a tautomer, isotopolog, or pharmaceutically acceptable salt thereof; Compound 2, or a tautomer, isotopolog, or pharmaceutically acceptable salt thereof; or Compound 3, or an enantiomer, a mixture of enantiomers, tautomer, isotopolog or pharmaceutically acceptable salt thereof. It is to be understood that the compounds provided herein may contain chiral centers. Such chiral centers may be of either the (R) or (S) configuration, or may be a mixture thereof. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form. Optically active (+) and (−), (R)- and (S)-, or (D)- and (L)-isomers may be prepared using chiral synthons or chiral reagents, or resolved using conventional techniques, such as chromatography on a chiral stationary phase. In the description herein, if there is any discrepancy between a chemical name and chemical structure, the structure controls.

As used herein, the term “tautomer” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

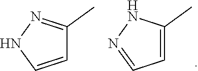

Unless specifically stated otherwise, where a compound may assume alternative tautomeric, regioisomeric and/or stereoisomeric forms, all alternative isomers are intended to be encompassed within the scope of the claimed subject matter. For example, where a compound can have one of two tautomeric forms, it is intended that both tautomers be encompassed herein. Thus, the compounds provided herein may be enantiomerically pure, or be stereoisomeric or diastereomeric mixtures. As used herein and unless otherwise indicated, the term “enantiomerically pure” means a stereomerically pure composition of a compound having one chiral center.

As used herein and unless otherwise indicated, the term “stereoisomer” or “stereoisomerically pure” means one stereoisomer of a compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments provided herein, including mixtures thereof.

The use of stereoisomerically pure forms of such compounds, as well as the use of mixtures of those forms, are encompassed by the embodiments provided herein. For example, mixtures comprising equal or unequal amounts of the enantiomers of a particular compound may be used in methods and compositions provided herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); Wilen, S. H., Tables of Resolving Agents and Optical Resolutions p. 268 (E. L. Eliel, Ed., Univ. of Notre Dame Press, Notre Dame, Ind., 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuj a, S., Chiral Separation Methods for Pharmaceutical and Biotechnological Products (John Wiley & Sons, 2011).

As used herein, an “isotopolog” or “isotopologue” refers to an isotopically enriched compound. The term “isotopically enriched” refers to an atom or compound having an isotopic composition other than the natural isotopic composition of that atom or compound. Radiolabeled and isotopically enriched compounds are useful as therapeutic agents, e.g., a hematological cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. Exemplary isotopologs include deuterium, carbon-13, or nitrogen-15 enriched compounds. For example, an isotopolog can be a deuterium enriched compound, such as Compound 1, 2, or 3, where the deuteration occurs on the chiral center.

It should be noted that if there is a discrepancy between a depicted structure and a name given to that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

As used herein and unless otherwise indicated, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of a compound provided herein include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p toluenesulfonic acid. Others are well-known in the art, see for example, Remington's Pharmaceutical Sciences, 18th eds., Mack Publishing, Easton Pa. (1990) or Remington: The Science and Practice of Pharmacy, 19th eds., Mack Publishing, Easton Pa. (1995).

The term “sample” as used herein relates to a material or mixture of materials, typically, although not necessarily, in fluid form, containing one or more components of interest. An exemplary sample is a “biological sample” obtained from a biological subject, including a sample of biological tissue or fluid origin, obtained, reached, or collected in vivo or in situ. A biological sample also includes samples from a region of a biological subject containing pre-cancerous or cancer cells or tissues. Such samples can be, but are not limited to, organs, tissues, and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include, but are not limited to, whole blood, partially purified blood, PBMC, tissue biopsies, including bone marrow core biopsy, bone marrow aspirate, isolated bone marrow mononuclear cells, circulating tumor cells and the like.

A “biological marker” or “biomarker” is a substance whose detection indicates a particular biological state, such as, for example, the presence of a hematological cancer. In some embodiments, biomarkers can be determined individually. In other embodiments, several biomarkers can be measured simultaneously.

In some embodiments, a “biomarker” indicates a change in the level of mRNA expression that may correlate with the risk or progression of a disease, or with the susceptibility of the disease to a given treatment. In some embodiments, the biomarker is a nucleic acid, such as mRNA or cDNA.

In additional embodiments, a “biomarker” indicates a change in the level of polypeptide or protein expression that may correlate with the risk or progression of a disease, or patient's susceptibility to treatment. In some embodiments, the biomarker can be a polypeptide or protein, or a fragment thereof. The relative level of specific proteins can be determined by methods known in the art. For example, antibody based methods, such as an immunoblot, enzyme-linked immunosorbent assay (ELISA), or other methods can be used.

The terms “polypeptide” and “protein,” as used interchangeably herein, refer to a polymer of three or more amino acids in a serial array, linked through peptide bonds. The term “polypeptide” includes proteins, protein fragments, protein analogues, oligopeptides, and the like. The term “polypeptide” as used herein can also refer to a peptide. The amino acids making up the polypeptide may be naturally derived, or may be synthetic. The polypeptide can be purified from a biological sample. The polypeptide, protein, or peptide also encompasses modified polypeptides, proteins, and peptides, e.g., glycopolypeptides, glycoproteins, or glycopeptides; or lipopolypeptides, lipoproteins, or lipopeptides.

As used herein, the term “level” refers to the amount, accumulation, or rate of a biomarker molecule. A level can be represented, for example, by the amount or the rate of synthesis of a messenger RNA (mRNA) encoded by a gene, the amount or the rate of synthesis of a polypeptide or protein encoded by a gene, or the amount or the rate of synthesis of a biological molecule accumulated in a cell or biological fluid. The term “level” refers to an absolute amount of a molecule in a sample or a relative amount of the molecule, determined under steady-state or non-steady-state conditions.

An “altered level” is intended to mean an amount, accumulation, or rate of a biomarker molecule that is different relative to a particular reference. An altered level can either be a decrease, or an increase, depending on the particular biomarker and/or the reference that is being used for comparison. For example, a biomarker level, such as a protein level, can be an altered level if it is decreased in a sample following administration of a treatment compound relative to an untreated sample. However, that same biomarker, could have an altered level that is increased if, for example, the reference level is a treated sample at an earlier time point.

As used herein, the term “reference level” is intended to mean a control level of a biomarker used to evaluate a test level of the biomarker in a sample from an individual. A reference level can be a normal reference level in a sample from a normal subject or a disease reference level from a disease-state subject. A normal reference level is an amount of expression of a biomarker in a non-diseased subject or subjects (i.e., hematological cancer-free). A disease-state reference level is an amount of expression of a biomarker in a subject with a positive diagnosis for the disease or condition. A reference level also can be a stage-specific reference level. A stage-specific reference level refers to a level of a biomarker characteristic of a given stage of progression of a disease or condition. A reference level can also be an amount of expression of a biomarker prior to treatment, or at a different time during treatment. For example, a reference level can be the amount of expression of a biomarker in the bone marrow prior to treatment. In another example, a reference level may be the expression of a biomarker in the blood at some point during or after treatment.

As used herein, the term “detectable” when used in reference to a biomarker is intended to mean that the amount of a biomarker is above the recognition threshold using known techniques for detection of biological molecules, such as immunochemical or histological methods. For example, a detectable biomarker level using an immunoblot can be a level that is above the background and/or the level of a negative control (e.g., no sample). Alternatively, a detectable biomarker level using, for example, quantitative RT-PCR (qPCR) can be a level that is detected at an earlier cycle number than the cycle number of detection for a qPCR reaction using a negative control (e.g., water). It is further understood that a detectable level can refer to qualitatively or quantitatively determining the presence or concentration of the biomolecule under investigation, and that the assays described above are merely exemplary.

As used herein, the terms “predict” or “predicting,” generally mean to determine or tell in advance. When used to “predict” the responsiveness of a treatment, for example, the term “predicting” can mean that the likelihood of responding, or not responding, to the hematological cancer treatment can be determined at the outset, before the treatment has begun, or before the treatment period has progressed substantially.

As used herein, the terms “monitor” or “monitoring,” generally refer to the overseeing, supervision, regulation, watching, tracking, or surveillance of an activity. For example, the term “monitoring the effectiveness of a compound” refers to tracking the effectiveness in treating a hematological cancer in a patient or in a tumor cell culture. Similarly, the term “monitoring,” when used in connection with patient compliance, either individually, or in a clinical trial, refers to the tracking or confirming that the patient is actually taking a drug being tested as prescribed. The monitoring can be performed, for example, by following the expression of mRNA or protein biomarkers.

As used herein, the term “efficacy” refers to the ability to produce a desired or intended result. When used in reference to the efficacy of a treatment compound, “efficacy” is intended to mean a decrease or inhibition in the growth, or progression of a hematological cancer, such as DLBCL or CLL/SLL. It can also refer to prevention of a recurrence or relapse in a hematological cancer, such as DLBCL or CLL/SLL.

As used herein, the term “time points” refers to samples that are obtained at separate intervals that are spaced sufficiently in time to allow for a response, if one is expected. A time point can be before, during, or after treatment. It is understood that multiple time points can be taken at each stage of a treatment cycle. For example, a sample can be obtained more than a month before treatment, and again immediately prior to treatment, or twice a day during treatment, or before, during and after treatment. It is understood that the examples provided above are merely exemplary and are not intended to be limiting.

As used herein, and unless otherwise specified, the terms “therapeutically effective amount” and “effective amount” of a compound refer to an amount sufficient to provide a therapeutic benefit in the treatment, prevention and/or management of a disease, for example DLBCL or CLL/SLL, or to delay or minimize one or more symptoms associated with the disease or disorder to be treated. The terms “therapeutically effective amount” and “effective amount” can encompass an amount that improves overall therapy, reduces or avoids symptoms or causes of disease or disorder, or enhances the therapeutic efficacy of another therapeutic agent.

As used herein and unless otherwise indicated, the terms “treat,” “treating” and “treatment” refer to alleviating in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself, for example, a hematological cancer such as DLBCL or CLL/SLL.

As used herein and unless otherwise indicated, the term “preventing” means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition.

As used herein and unless otherwise indicated, the term “managing” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

As described herein, the terms “near,” “about,” or “approximately” refer to a level that is within a close range of the reference level. The biomarker level that is at or near a reference level can be lower or higher than the reference level, such that it is within a range of the range of 50% or greater than the reference level. Those skilled in the art will understand that a biomarker level need not be equal to the reference biomarker level to be considered at or near the reference biomarker level. An exemplary biomarker level that is at or near the biomarker level of a reference sample can be within 75-125% of the reference level.

As described herein, the term “neutrophil” refers to differentiated myeloid cells. Neutrophils can be characterized by expression of the surface marker CD11b, and the absence or near absence of the surface markers CD34 and CD33 (i.e., CD11b⁺, CD34⁻, and CD33⁻). Those skilled in the art will understood that expression or lack of expression of a marker need not be absolute. For example, a neutrophil may express low levels of CD34 and be characterized as CD34⁻. Similarly, a cell may express moderate, but detectable levels of CD11b, and be characterized as CD11b⁺. Expression levels can be determined empirically by both the individual, and the instrument being used to measure the markers.

As described herein, the term “second active agent” refers to any additional treatment that is biologically active. It is understood that the second active agent can be a hematopoietic growth factor, cytokine, anti-cancer agent, antibiotic, cox-2 inhibitor, immunomodulatory agent, immunosuppressive agent, corticosteroid, therapeutic antibody that specifically binds to a cancer antigen or a pharmacologically active mutant, or derivative thereof. Exemplary second active agents include, but are not limited to, an HDAC inhibitor (e.g., panobinostat, romidepsin, vorinostat, or citarinostat), a BCL2 inhibitor (e.g., venetoclax), a BTK inhibitor (e.g., ibrutinib or acalabrutinib), an mTOR inhibitor (e.g., everolimus), a PI3K inhibitor (e.g., idelalisib), a PKCβ inhibitor (e.g., enzastaurin), a SYK inhibitor (e.g., fostamatinib), a JAK2 inhibitor (e.g., fedratinib, pacritinib, ruxolitinib, baricitinib, gandotinib, lestaurtinib, or momelotinib), an Aurora A kinase inhibitor (e.g., alisertib), an EZH2 inhibitor (e.g., tazemetostat, GSK126, CPI-1205, 3-deazaneplanocin A, EPZ005687, EI1, UNC1999, or sinefungin), a BET inhibitor (e.g., birabresib or 4-[2-(cyclopropylmethoxy)-5-(methanesulfonyl)phenyl]-2-methylisoquinolin-1(2H)-one), a hypomethylating agent (e.g., 5-azacytidine or decitabine), a chemotherapy (e.g., bendamustine, doxorubicin, etoposide, methotrexate, cytarabine, vincristine, ifosfamide, melphalan, oxaliplatin, or dexamethasone), or an epigenetic compound (e.g., a DOT1L inhibitor such as pinometostat, a HAT inhibitor such as C646, a WDR5 inhibitor such as OICR-9429, a DNMT1 selective inhibitor such as GSK3484862, a LSD-1 inhibitor such as Compound C or seclidemstat, a G9A inhibitor such as UNC0631, a PRMT5 inhibitor such as GSK3326595, a bromodomain (BRD) inhibitor (e.g., a BRD9/7 inhibitor such as LP99), a SUV420H1/H2 inhibitor such as A-196, or a CARM1 inhibitor such as EZM2302).

As used herein, the term “supportive care therapy” refers to any substance that treats, prevents or manages an adverse effect from treatment with Compound 1, Compound 2 or Compound 3, or an enantiomer or a mixture of enantiomers, tautomers, isotopolog or a pharmaceutically acceptable salt thereof. It is understood that the term “support care therapy” refers to any therapeutic agent that is mainly directed to sustaining the strength and/or comfort of the patient Exemplary support care therapies include, but are not limited to, therapies for pain control, intravenous fluids, and electrolyte support, such as isotonic saline, glucose saline, or balanced crystalloid solutions.

As used herein, the term “source” when used in reference to a reference sample refers to the origin of a sample. For example, a sample that is taken from blood would have a reference sample that is also taken from blood. Similarly, a sample that is taken from bone marrow would have a reference sample that is also taken from the bone marrow.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from the RNA molecule to give a protein, a polypeptide, or a portion thereof.

The terms “cereblon” or “CRBN” and similar terms refers to the polypeptides (“polypeptides,” “peptides,” and “proteins” are used interchangeably herein) comprising the amino acid sequence of any CRBN, such as a human CRBN protein (e.g., human CRBN isoform 1, GenBank Accession No. NP_057386; or human CRBN isoforms 2, GenBank Accession No. NP_001166953, each of which is herein incorporated by reference in its entirety), and related polypeptides, including SNP variants thereof. Related CRBN polypeptides include allelic variants (e.g., SNP variants), splice variants, fragments, derivatives, substitution variant, deletion variant, insertion variant, fusion polypeptides, and interspecies homologs, which, in certain embodiments, retain CRBN activity and/or are sufficient to generate an anti-CRBN immune response.

As used herein, the term “cereblon-associated protein” or “CAP” refers to a protein that interacts with or binds to cereblon (CRBN) directly or indirectly. For example, the term refers to any protein that directly binds to cereblon, as well as any protein that is an indirect downstream effector of CRBN pathways. An exemplary CAP is a substrate of CRBN, for example, a protein substrate of the E3 ubiquitin ligase complex involving CRBN, such as IKZF1, IKZF3, or ZFP91.

As used herein, the term “interferon inducible gene” refers to genes whose expression in increased in response to type I interferon-mediated signaling. For example, the binding of interferons (IFNs) to the type I IFN receptor can trigger the activation of a signaling cascade that is responsible for the induction of an interferon inducible gene. Exemplary genes include interferon regulatory 7 (IRF7), interferon induced protein with tetratricopeptide repeats 3 (IFIT3), and Dead box protein 58 (DDX58).

As used herein, the term “associated with” when used in reference to a signaling pathway, cellular process, or cellular feature, is intended to mean that the molecule is a member of a group of molecules in a cell that work together, for example, to control a specific process or function. It is understood that a molecule can be associated with a signaling pathway because it directly or indirectly participates in propogating transduction of a signal, such as interferon signaling or cytokine/chemokine signaling. A molecule can also be associated with a cellular process or feature, such as, for example, cell adhesion, cell-cell junction, G-protein coupled receptor, extracellular matrix, cell cycle, or transcription, because the molecule participates directly or indirectly in that cellular process or feature.

As used herein, the terms “T-cell activation” and “activated T-cell” are intended to mean cellular activation of resting naïve T-cells into effector T-cells that are capable of inducing tumor cell death. T-cell activation can be initiated by the interaction of the T-cell receptor (TCR)/CD3 complex with an antigen. An exemplarity activated T cell exhibits cell responses that include, but are not limited to, cell proliferation, cytokine secretion, and/or effector function. In the context of the present application, T-cell activation may be induced by treatment with Compound 1, or a tautomer, isotopolog, or pharmaceutically acceptable salt thereof; Compound 2, or a tautomer, isotopolog, or pharmaceutically acceptable salt thereof; or Compound 3, or an enantiomer, a mixture of enantiomers, tautomer, isotopolog or pharmaceutically acceptable salt thereof.

As used herein, the term “T-cell activation associated cytokine” refers to any of the numerous factors that are secreted by activated T-cell, or whose secretion increases in activated T-cells, relative to resting naïve T-cells. An exemplary T-cell activation associated cytokines includes interleukin-2 (IL-2).

The terms “antibody,” “immunoglobulin,” or “Ig” as used interchangeably herein, encompasses fully assembled antibodies and antibody fragments that retain the ability to specifically bind to the antigen. Antibodies provided herein include, but are not limited to, synthetic antibodies, monoclonal antibodies, polyclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above. In particular, antibodies provided herein include immunoglobulin molecules and immunologically active portions of immunoglobulin molecules, i.e., antigen binding domains or molecules that contain an antigen-binding site that immunospecifically binds to CRBN antigen (e.g., one or more complementarity determining regions (CDRs) of an anti-CRBN antibody). Immunoglobulins can be composed of heavy chains and light chains. The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. In some embodiments, the anti-CRBN antibodies are fully human, such as fully human monoclonal CRBN antibodies. In certain embodiments, antibodies provided herein are IgG antibodies, or a subclass thereof (e.g., human IgG1 or IgG4). In other embodiments, the antibodies provided herein include immunoglobulin molecules and immunologically active portions of immunoglobulin molecules, i.e., antigen binding domains or molecules that contain an antigen-binding site that immunospecifically binds to, for example, Aiolos, Ikaros, c-MYC, IRF4, Caspase-3, or any of the biomarkers provided herein.

As used herein, the terms “immunospecifically binds,” “immunospecifically recognizes,” “specifically binds,” and “specifically recognizes” are analogous terms in the context of antibodies and refer to molecules that bind to an antigen/epitope as such binding is understood by one skilled in the art. Antibodies that specifically bind to a target structure, or subunit thereof, do not cross-react with biological molecules that are outside the target structure family. In some embodiments, an antibody or antibody fragment binds to a selected antigen with a specific affinity of greater than 10⁻⁷ M, 10⁻⁸M, 10⁻⁹M, 10⁻¹⁰ M, or 10⁻¹¹ M, between 10⁻⁸ M-10⁻¹¹ M, 10⁻⁹ M-10⁻¹⁰ M, and 10⁻¹⁰ M-10⁻¹¹ M. For example, a molecule (e.g., an antibody) that specifically binds to an antigen may bind to other peptides or polypeptides, generally with lower affinity as determined by, e.g., immunoassays, or other assays known in the art. In a specific embodiment, molecules that specifically bind to an antigen do not cross react with other proteins.

The term “epitope” as used herein refers to a localized region on the surface of an antigen that is capable of binding to one or more antigen binding regions of an antibody, that has antigenic or immunogenic activity in an animal, such as a mammal (e.g., a human), and that is capable of eliciting an immune response. An epitope having immunogenic activity is a portion of a polypeptide that elicits an antibody response in an animal. An epitope having antigenic activity is a portion of a polypeptide to which an antibody immunospecifically binds as determined by any method well known in the art, for example, by the immunoassays described herein. Antigenic epitopes need not necessarily be immunogenic. Epitopes usually consist of chemically active surface groupings of molecules, such as amino acids or sugar side chains, and have specific three dimensional structural characteristics as well as specific charge characteristics. A region of a polypeptide contributing to an epitope may be contiguous amino acids of the polypeptide, or the epitope may come together from two or more non-contiguous regions of the polypeptide. The epitope may or may not be a three-dimensional surface feature of the antigen.

The terms “determining,” “measuring,” “evaluating,” “assessing,” and “assaying” as used herein generally refer to any form of measurement, and include determining whether an element is present or not. These terms include quantitative and/or qualitative determinations. Assessing may be relative or absolute. “Assessing the presence of” can include determining the amount of something present, as well as determining whether it is present or absent.

As described herein, the term “detectable label” refers to the attachment of a specific tag to an antibody to aid in the detection or isolation/purification of a protein. Examples of types of labels include, but are not limited to, a radioisotope, a fluorophore (e.g., fluorescein isothiocyanate (FITC), phycoerythrin (PE)), chemiluminescence, enzyme reporters, and element particles (e.g., gold particles). Detection can be direct or indirect Optical methods include microscopy (both confocal and non-confocal), imaging methods and non-imaging methods. Electrochemical methods include voltammetry and amperometry methods. Radio frequency methods include multipolar resonance spectroscopy.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual (2d ed. 1989); Glover, ed., DNA Cloning, Volumes I and II (1985); Gait, ed., Oligonucleotide Synthesis (1984); Hames & Higgins, eds., Nucleic Acid Hybridization (1984); Hames & Higgins, eds., Transcription and Translation (1984); Freshney, ed., Animal Cell Culture: Immobilized Cells and Enzymes (IRL Press, 1986); Immunochemical Methods in Cell and Molecular Biology (Academic Press, London); Scopes, Protein Purification: Principles and Practice (Springer Verlag, N.Y., 2d ed. 1987); and Weir & Blackwell, eds., Handbook of Experimental Immunology, Volumes I-IV (1986).

5.2 Compounds

In some embodiments of the various methods provided herein, are compounds of Formula (I):

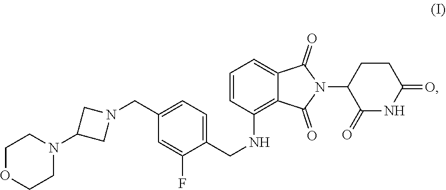 [SMILES: O=C1CCC(N2C(=O)c3cccc(NCc4ccc(CN5CC(N6CCOCC6)C5)cc4F)c3C2=O)C(=O)N1]

or an enantiomer or a mixture of enantiomers, tautomer, isotopolog or pharmaceutically acceptable salt thereof.

In certain embodiments of the various methods provided herein, the compound is (S)-2-(2,6-dioxopiperidin-3-yl)-4-((2-fluoro-4-((3-morpholinoazetidin-1-yl)methyl)benzyl)amino)isoindoline-1,3-dione (Compound 1):

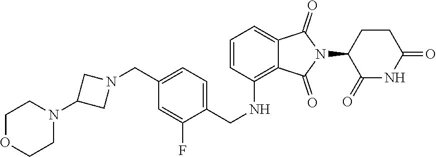 [SMILES: O=C1CC[C@H](N2C(=O)c3cccc(NCc4ccc(CN5CC(N6CCOCC6)C5)cc4F)c3C2=O)C(=O)N1]

or a tautomer, isotopolog, or pharmaceutically acceptable salt thereof. Methods of preparing Compound 1 are described in U.S. application Ser. No. 16/390,815, which is incorporated herein by reference in its entirety.

In yet other embodiments of the various methods provided herein, the compound is (R)-2-(2,6-dioxopiperidin-3-yl)-4-((2-fluoro-4-((3-morpholinoazetidin-1-yl)methyl)benzyl)amino)isoindoline-1,3-dione (Compound 2):

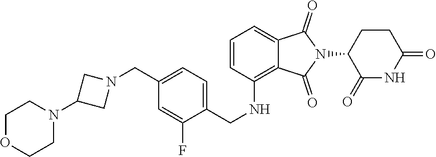 [SMILES: O=C1CC[C@@H](N2C(=O)c3cccc(NCc4ccc(CN5CC(N6CCOCC6)C5)cc4F)c3C2=O)C(=O)N1]

or a tautomer, isotopolog or pharmaceutically acceptable salt thereof.

In some embodiments, the compound comprises a mixture of (S)-2-(2,6-dioxopiperidin-3-yl)-4-((2-fluoro-4-((3-morpholinoazetidin-1-yl)methyl)benzyl)amino)isoindoline-1,3-dione, and (R)-2-(2,6-dioxopiperidin-3-yl)-4-((2-fluoro-4-((3-morpholinoazetidin-1-yl)methyl)benzyl)amino)isoindoline-1,3-dione (Compound 3):

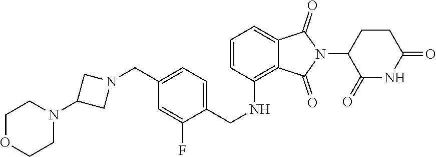 [SMILES: O=C1CCC(N2C(=O)c3cccc(NCc4ccc(CN5CC(N6CCOCC6)C5)cc4F)c3C2=O)C(=O)N1]

or an enantiomer, mixture of enantiomers, tautomer, isotopolog, or pharmaceutically acceptable salt thereof.

The various compounds provided herein may contain chiral centers, and can exist as mixtures of enantiomers (e.g., racemic mixtures) or mixtures of diastereomers. Such chiral centers may be of either the (R) or (S) configuration, or may be a mixture thereof. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form. The methods provided herein encompass the use of stereomerically pure forms of such compounds as well as mixtures of those forms. For example, mixtures comprising equal or unequal amounts of the enantiomers of a particular compound may be used in methods provided herein. These isomers may be asymmetrically synthesized or resolved using standard techniques, such as chiral columns or chiral resolving agents. See, Jacques et al., Enantiomers, Racemates and Resolutions (Wiley-Interscience, New York, 1981); Wilen et al., Tetrahedron 1977, 33:2725-2736; Eliel, Stereochemistry of Carbon Compounds (McGraw-Hill, N Y, 1962); Wilen, Tables of Resolving Agents and Optical Resolutions, p. 268 (Eliel, ed., Univ. of Notre Dame Press, Notre Dame, Ind., 1972).

Also provided herein are isotopically enriched analogs of the compounds provided herein. Isotopic enrichment (for example, deuteration) of pharmaceuticals to improve pharmacokinetics (“PK”), pharmacodynamics (“PD”), and toxicity profiles, has been demonstrated previously with some classes of drugs. See, e.g., Lijinsky et. al., Food Cosmet. Toxicol., 20: 393 (1982); Lijinsky et. al., J. Nat. Cancer Inst., 69: 1127 (1982); Mangold et. al., Mutation Res. 308: 33 (1994); Gordon et. al., Drug Metab. Dispos., 15: 589 (1987); Zello et. al., Metabolism, 43: 487 (1994); Gately et. al., J Nucl. Med., 27: 388 (1986); Wade D, Chem. Biol. Interact. 117: 191 (1999).

Without being limited by any particular theory, isotopic enrichment of a drug can be used, for example, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrease the production of deleterious metabolites in specific tissues and/or create a more effective drug and/or a safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen will cause a decrease in the reaction rate and the process will slow down. This phenomenon is known as the Deuterium Kinetic Isotope Effect (“DKIE”). (See, e.g., Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. Without being limited by a particular theory, high DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T20. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects.

Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, will provide a similar kinetic isotope effects.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. As a result, these drugs often require the administration of multiple or high daily doses.

Isotopic enrichment at certain positions of a compound provided herein may produce a detectable KIE that affects the pharmacokinetic, pharmacologic, and/or toxicological profiles of a compound provided herein in comparison with a similar compound having a natural isotopic composition. In one embodiment, the deuterium enrichment is performed on the site of C—H bond cleavage during metabolism.

Standard physiological, pharmacological and biochemical procedures are available for testing the compounds to identify those that possess the desired anti-proliferative activity.

Such assays include, for example, biochemical assays such as binding assays, radioactivity incorporation assays, as well as a variety of cell based assays.

Compounds of Formula (I) or a pharmaceutically acceptable salt, solvate, hydrate, stereoisomer, tautomer or racemic mixtures thereof can be prepared by methods known to one of skill in the art, for example, according to the procedure described in U.S. Pat. No. 8,518,972 B2, or U.S. application Ser. No. 16/390,815, which are each incorporated herein by reference in their entirety. Exemplary methods for preparation of the compounds provided herein are described in Examples 1-3 of Section 6.

### 5.3 Biomarkers and Methods of Use Thereof

The methods provided herein are based, in part, on the finding that a detectable increase or decrease in certain biomarkers upon compound treatment are observed in subjects with a hematological cancer, such as for example DLBCL or CLL/SLL, who are responsive to a given treatment, e.g., a compound, such as Compound 1, Compound 2, or Compound 3, or an enantiomer, a mixture of enantiomers, tautomer, isotopolog or pharmaceutically acceptable salt thereof as described in Section 5.2 above. The levels of these biomarkers may be used for identifying or measuring the responsiveness of the subjects to the treatment, as well as facilitate the treatment of a subject having a hematological cancer. In some embodiments, the levels of biomarkers can be predictive of response to a compound of Formula (I) or an enantiomer, mixture of enantiomers, tautomer, isotopolog, or pharmaceutically acceptable salt thereof. In some embodiments, the compound of Formula (I) comprises a compound selected from the group consisting (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof; (R)-2-(2,6-Dioxopiperidin-3-yl)-4-((2-fluoro-4-((3-morpholinoazetidin-1-yl)methyl)benzyl)amino)isoindoline-1,3-dione (Compound 2), or a tautomer, isotopolog, or pharmaceutically acceptable salt thereof; or 2-(2,6-Dioxopiperidin-3-yl)-4-((2-fluoro-4-((3-morpholinoazetidin-1-yl)methyl)benzyl)amino)isoindoline-1,3-dione (Compound 3), or an enantiomer, mixture of enantiomers, tautomer, isotopolog, or pharmaceutically acceptable salt thereof. In certain embodiments, the treatment compound of Formula (I) is (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof. In some embodiments, the hematological cancer comprises non-Hodgkin's lymphoma. In specific embodiments, the non-Hodgkin's lymphoma is DLBCL. In yet further embodiments, the DLBCL is relapsed, refractory, or resistant to conventional therapy. In some embodiments, the hematological cancer is CLL/SLL. In yet further embodiments, the CLL/SLL is relapsed, refractory, or resistant to conventional therapy.

As described in the Examples in Section 6, and shown in the figures, the levels of certain proteins, molecules, mRNAs, or cell composition change in response to treatment with a compound of Formula (I) or an enantiomer, mixture of enantiomers, tautomer, isotopolog, or pharmaceutically acceptable salt thereof. These biomarkers include markers of apoptosis, cereblon (CRBN)-associated proteins, and interferon inducible genes. For example, as provided herein, treatment of DLBCL cells that were sensitive to a compound of Formula (I), such as Compound 1, Compound 2, or Compound 3, induced apoptosis, decreased expression of CRBN-associated proteins, and increased expression of interferon inducible genes, relative to untreated cells and nonresponsive cells. However, it is understood that in certain circumstances the biomarkers described herein need not be an altered level (i.e., increase or decrease). For example, in certain embodiments the basal expression of a protein prior to administering a dosage of a treatment compound to a subject can predict sensitivity or responsiveness to treatment with a compound of Formula (I) or an enantiomer, mixture of enantiomers, tautomer, isotopolog, or pharmaceutically acceptable salt thereof. Therefore, the biomarkers provided herein can be useful in identifying or measuring the responsiveness of the subjects to the treatment, monitoring efficacy of treatment, as well as facilitate the treatment of a subject having a hematological cancer, such as DLBCL or CLL/SLL.

In certain aspects, the biomarker useful in the methods provided herein is a marker of apoptosis. As provided herein, treatment of a hematological cancer cells, such as DLBCL or CLL/SLL cells, that are sensitive to a compound of Formula (I) or an enantiomer, mixture of enantiomers, tautomer, isotopolog, or pharmaceutically acceptable salt thereof can increase the expression and/or increase the activity of pro-apoptotic proteins, decrease the expression and/or activity of anti-apoptotic proteins, and result in the induction of apoptosis. Accordingly, in some embodiments the alteration of the biomarker level is indicative of the induction of apoptosis. In specific embodiments, the biomarker indicative of the induction of apoptosis is cleaved caspase 3, cleaved caspase 7, cleaved poly (ADP-ribose) polymerase (PARP), BCL2, survivin, phosphatidylserine (PS) and DNA, Bcl-2-like protein 11 (BIM), interleukin 27 (IL27), tumor necrosis factor (TNF), interleukin 10 (IL10), or a combination thereof. The biomarker that is indicative of the induction of apoptosis can also be detection of a probe or surrogate marker, such as Annexin-V, Deep Red Anthraquinone 7 (DRAQ7), and/or 7-amino-actinomycin D (7-AAD), that bind to endogenous proteins and molecules (e.g., phosphatidylserine, DNA, respectively) in apoptotic cells and whose detection distinguishes viable, apoptotic, and late apoptotic/dead cells. Thus, in some embodiments, the biomarker that is indicative of the induction of apoptosis includes Annexin-V, DRAQ7, 7-AAD, or a combination thereof.

In some embodiments, the biomarker indicative of the induction of apoptosis in the sample is higher than a reference level of the biomarker. For example, in certain embodiments, the reference level is the level of the biomarker prior to the administration of the compound, and a higher level of the biomarker indicative of the induction of apoptosis post administration as compared to the reference level is indicative of effectiveness of the treatment. In specific embodiments, the reference level is the level of the biomarker prior to the administration of the compound, and a higher level of cleaved caspase 3, cleaved caspase 7, cleaved poly (ADP-ribose) polymerase (PARP), BCL2, survivin, phosphatidylserine (PS) and DNA, Bcl-2-like protein 11 (BIM), interleukin 27 (IL27), tumor necrosis factor (TNF), interleukin 10 (IL10), Annexin-V, DRAQ7, 7-AAD, or a combination thereof in the sample post administration as compared to the reference level is indicative of effectiveness of the treatment. However, it is understood that the biomarker in the sample need not be higher than the reference level of the biomarker. Therefore, in some embodiments, the biomarker in the sample is lower than the reference level of the biomarker. For example, in some embodiments, the reference level is the level of the biomarker post a first administration of the compound, and a lower level of the biomarker indicative of the induction of apoptosis prior to a second administration is informative for adjusting a dosage amount or frequency for treating a subject having a hematological cancer. In one embodiment, the hematological cancer is DLBCL. In another embodiment, the hematological cancer is CLL/SLL.

In some aspects, the biomarker useful in the methods provided herein is a CRBN-associated protein or a transcriptional target of a CRBN-associated protein. As provided herein, the compound of Formula (I) or an enantiomer, mixture of enantiomers, tautomer, isotopolog, or pharmaceutically acceptable salt thereof is able to bind to CRBN, and CRBN expression is required to mediate the effects of treatment with a compound of Formula (I) or an enantiomer, mixture of enantiomers, tautomer, isotopolog, or pharmaceutically acceptable salt thereof. According, in some embodiments, the biomarker is cereblon (CRBN), and the subject is diagnosed as being likely to be responsive to treatment with a compound of Formula (I) or an enantiomer, mixture of enantiomers, tautomer, isotopolog, or pharmaceutically acceptable salt thereof if CRBN is detectable or higher than a reference level in the sample.

CRBN is an E3 ubiquitin ligase known to promote the degradation of various substrates including Ikaros (also known as IKZF1), Aiolos (also known as (IKZF3), and ZFP91 (see, e.g., U.S. Pat. No. 9,857,359 B2; U.S. application Ser. Nos. 15/101,869, and 15/518,472, published as U.S. 2017-0242014A1 and U.S. 2016-0313300A1, respectively, each of which is incorporated herein by reference in its entirety). As provided herein, treatment of a hematological cancer cells with a compound of Formula (I) led to the degradation of the CRBN associated proteins Ikaros, Aiolos, and ZFP91, which coincided with the strong antiproliferative effects of the compound of Formula (I). In addition, treatment of a hematological cancer cells with a compound of Formula (I) resulted in the de-repression of interferon inducible genes (ISGs), interferon regulatory factor 7 (IRF7), interferon-induced protein with tetratricopeptide repeats 3 IFIT3), and DExD/H-box helicase (DDX58), as well as the reduction of the highly critical transcription factors c-Myc/MYC, BCL6, and IRF4. Thus, in some embodiments, the biomarker is a CRBN-associated protein (CAP) or a transcriptional target of a CRBN-associated protein. In specific embodiments, the CRBN-associated protein is IKAROS, AIOLOS, or ZFP91. In other embodiments, the transcriptional target of a CRBN-associated protein is BCL6, c-MYC, or IRF4.

In some embodiments, the biomarker is a CRBN-associated protein (CAP) or a transcriptional target of a CRBN-associated protein and the biomarker in the sample is lower than a reference level of the biomarker. For example, in certain embodiments, the reference level is the level of the biomarker prior to the administration of the compound, and a lower level of the CRBN-associated protein (CAP) or the transcriptional target of a CRBN-associated protein post administration as compared to the reference level is indicative of effectiveness of the treatment. In specific embodiments, the reference level is the level of the biomarker prior to the administration of the compound, and a lower level of Ikaros, Aiolos, ZFP91, BCL6, c-MYC, IRF4, or a combination thereof in the sample post administration as compared to the reference level is indicative of effectiveness of the treatment. However, it is understood that the biomarker in the sample need not be lower than the reference level of the biomarker. Therefore, in some embodiments, the biomarker in the sample is higher than the reference level of the biomarker. For example, in some embodiments, the reference level is the level of the biomarker post a first administration of the compound, and a higher level of the biomarker is informative for adjusting a dosage amount or frequency for treating a subject having a hematological cancer. In one embodiment, the hematological cancer is DLBCL. In another embodiment, the hematological cancer is CLL/SLL

In some embodiments, the biomarker comprises an interferon inducible gene. In specific embodiments, the interferon inducible gene includes interferon regulatory 7 (IRF7), interferon induced protein with tetratricopeptide repeats 3 (IFIT3), DEAD box protein 58 (DDX58), or a combination thereof. In certain embodiments, the biomarker is an interferon inducible gene and the biomarker in the sample is higher than a reference level of the biomarker. For example, in certain embodiments, the reference level is the level of the biomarker prior to the administration of the compound, and a higher level of the interferon inducible gene post administration as compared to the reference level is indicative of effectiveness of the treatment. In specific embodiments, the reference level is the level of the biomarker prior to the administration of the compound, and a higher level of IRF7, IFIT3, DDX58, or a combination thereof in the sample post administration as compared to the reference level is indicative of effectiveness of the treatment. However, it is understood that the biomarker in the sample need not be higher than the reference level of the biomarker. Therefore, in some embodiments, the biomarker in the sample is lower than the reference level of the biomarker. For example, in some embodiments, the reference level is the level of the biomarker post a first administration of the compound, and a lower level of the biomarker is informative for adjusting a dosage amount or frequency for treating a subject having a hematological cancer. In one embodiment, the hematological cancer is DLBCL. In another embodiment, the hematological cancer is CLL/SLL.

As described herein, treatment of a hematological cancer cells with a compound of Formula (I) inhibited the proliferation of the hematological cancer cells, relative to non-treated cells. This was confirmed by an increase in expression of the proliferation inhibitor, cyclin dependent kinase inhibitor 1 (p21). Thus in some embodiments, the biomarker is a marker of proliferation. In specific embodiments, the biomarker is p21. It is understood that the biomarker can also be a marker of increased proliferation, and need not be a proliferation inhibitor. By way of example, the marker can be a marker of proliferation (e.g., Brdu, Ki-67, H3pS10 or similar marker) that decreases upon treatment with a compound of Formula (I) or an enantiomer, mixture of enantiomers, tautomer, isotopolog, or pharmaceutically acceptable salt thereof.

In some embodiments, the biomarker is a marker of proliferation and the biomarker in the sample is lower than a reference level of the biomarker. For example, in certain embodiments, the reference level is the level of the biomarker prior to the administration of the compound, and a lower level of the marker of proliferation post administration as compared to the reference level is indicative of effectiveness of the treatment. In specific embodiments, the reference level is the level of the biomarker prior to the administration of the compound, and a lower level of p21 in the sample post administration as compared to the reference level is indicative of effectiveness of the treatment. However, it is understood that the biomarker in the sample need not be lower than the reference level of the biomarker. Therefore, in some embodiments, the biomarker in the sample is higher than the reference level of the biomarker. For example, in some embodiments, the reference level is the level of the biomarker post a first administration of the compound, and a higher level of the marker of proliferation is informative for adjusting a dosage amount or frequency for treating a subject having a hematological cancer. In one embodiment, the hematological cancer is DLBCL. In another embodiment, the hematological cancer is CLL/SLL

The inventors have observed that a compound of Formula (I), or a pharmaceutically acceptable salt, solvate, hydrate, stereoisomer, tautomer or racemic mixtures thereof, can also affect non-cancer cells, such as endothelial cells, T and B lymphocytes, fibroblasts, and macrophages. The cells surrounding a malignant cell (i.e., tumor microenvironment) can impact the malignant cell. For example, anti-inflammatory and immunomodulatory signals can also assist in treating the hematological cancer. Thus, in some embodiments, the biomarker is in a non-cancer cell. In some embodiments, the biomarker is selected from a group consisting of IL-8, IL-1a, sPGE2, sTNFα, sIgG, sIL-17A, sIL-17F, sIL-2, sIL-6, collagen-I and -III, PAI-1, CD69, sIL-10, or a combination thereof. However, it is understood that the biomarker in the sample need not be lower than the reference level of the biomarker. Therefore, in some embodiments, the biomarker in the sample is higher than the reference level of the biomarker. In some embodiments, the biomarker is selected from a group consisting of IL-8, IL-1a, sPGE2, sTNFα, sIgG, sIL-17A, sIL-17F, sIL-2, sIL-6, collagen-I and -III, PAI-1, CD69, sIL-10 or a combination thereof and the biomarker in the sample is higher than a reference level of the biomarker. For example, in certain embodiments, the reference level is the level of the biomarker prior to the administration of the compound, and a lower level of IL-8, IL-1a, sPGE2, sTNFα, sIgG, sIL-17A, sIL-17F, CD-69, collagen-I and -III, PAI-1, or a combination thereof post administration as compared to the reference level is indicative of effectiveness of the treatment. In other embodiments, the reference level is the level of the biomarker prior to the administration of the compound, and a higher level of IL-8, sIL-10, sIL-2, sIL-6, or a combination thereof, in the sample post administration as compared to the reference level is indicative of effectiveness of the treatment.

It has also been observed that Compound 1 treatment produced an Ikaros/Aiolos-driven up-regulation of genes associated with interferon signalling (e.g., IL6ST, IFITM3, IFI6, OAS3, interferon α/β signaling), cytokine/chemokine signaling (e.g., IL23A, CCL1), apoptosis (e.g., IL27, TNF, IL10, caspase), cell adhesion (e.g., SELE, SELPLG, TXA2PA), cell-cell junction (e.g., CLDN7, CLDN12), G-protein coupled receptors (e.g., FFAR2), extracellular matrix (e.g., CD209, SERPINA, SERPINB7), and a global down-regulation of genes associated with cell cycle and transcription. Therefore, in some embodiments, the biomarker is associated with interferon signaling. In specific embodiments, the biomarker associated with interferon signaling includes interleukin-6 signal transducer (IL6ST), interferon-induced transmembrane protein 3 (IFITM3), interferon alpha-inducible protein 6 (IFI6), 2′-5′-oligoadenylate synthase 3 (OAS3), interferon α (IFNα), interferon β (IFN β), or a combination thereof. In specific embodiments, the biomarker is a marker of interferon signaling and the biomarker in the sample is higher than a reference level of the biomarker after treatment with a compound of Formula (I), or an enantiomer, mixture of enantiomers, tautomer, isotopolog, or pharmaceutically acceptable salt thereof, where the reference level is a DMSO treated sample.

In other embodiments, the biomarker is associated with cytokine/chemokine signaling. In some embodiments, the biomarker associated with cytokine/chemokine signaling includes interleukin-23 subunit alpha (IL23A), C—C motif chemokine 1 (CCL1), or a combination thereof. In specific embodiments, the biomarker is associated with cytokine/chemokine signaling and the biomarker in the sample is higher than a reference level of the biomarker after treatment with a compound of Formula (I), or an enantiomer, mixture of enantiomers, tautomer, isotopolog, or pharmaceutically acceptable salt thereof, where the reference level is a DMSO treated sample.

In further embodiments, the biomarker is associated with cell adhesion. In certain embodiments, the biomarker associated with cell adhesion includes E-selectin (SELE), P-selectin glycoprotein ligand 1 (SELPLG), thromboxane A2 (TXA2), or a combination thereof. In specific embodiments, the biomarker is associated with cell adhesion and the biomarker in the sample is higher than a reference level of the biomarker after treatment with a compound of Formula (I), or an enantiomer, mixture of enantiomers, tautomer, isotopolog, or pharmaceutically acceptable salt thereof, where the reference level is a DMSO treated sample.

In other embodiments, the biomarker is associated with cell-cell junction. In certain embodiments, the biomarker associated with cell-cell junction includes claudin 7 (CLDN7), claudin 12 (CLDN12), or a combination thereof. In specific embodiments, the biomarker is associated with cell-cell junction and the biomarker in the sample is higher than a reference level of the biomarker after treatment with a compound of Formula (I), or an enantiomer, mixture of enantiomers, tautomer, isotopolog, or pharmaceutically acceptable salt thereof 1, where the reference level is a DMSO treated sample.

In some embodiments, the biomarker is a G-protein coupled receptor. In certain embodiments, the G-protein coupled receptor includes free fatty acid receptor 2 (FFAR2). In specific embodiments, the biomarker is a G-protein coupled receptor and the biomarker in the sample is higher than a reference level of the biomarker after treatment with a compound of Formula (I), or an enantiomer, mixture of enantiomers, tautomer, isotopolog, or pharmaceutically acceptable salt thereof, where the reference level is a DMSO treated sample.

In certain embodiments, the biomarker is associated with the extracellular matrix. In some embodiments, the biomarker associated with the extracellular matrix includes CD209, SERPINA, SERPINB7, or a combination thereof. In specific embodiments, the biomarker is associated with extracellular matrix and the biomarker in the sample is higher than a reference level of the biomarker after treatment with a compound of Formula (I), or an enantiomer, mixture of enantiomers, tautomer, isotopolog, or pharmaceutically acceptable salt thereof, where the reference level is a DMSO treated sample.

In other embodiments, the biomarker is associated with cell cycle. In specific embodiments, the biomarker is associated with cell cycle and the biomarker in the sample is lower than a reference level of the biomarker after treatment with a compound of Formula (I), or an enantiomer, mixture of enantiomers, tautomer, isotopolog, or pharmaceutically acceptable salt thereof, where the reference level is a DMSO treated sample.

In further embodiments, the biomarker is associated with transcription. In specific embodiments, the biomarker is associated with transcription and the biomarker in the sample is lower than a reference level of the biomarker after treatment with a compound of Formula (I), or an enantiomer, mixture of enantiomers, tautomer, isotopolog, or pharmaceutically acceptable salt thereof, where the reference level is a DMSO treated sample.

In certain embodiments, the biomarker is the protein level of a protein that changes expression upon treatment with a compound of Formula (I), or an enantiomer, mixture of enantiomers, tautomer, isotopolog, or pharmaceutically acceptable salt thereof. In specific embodiments, the biomarker is one or more proteins selected from the group consisting of Aiolos (IKZF3), Ikaros (IKZF1), E3 ubiquitin-protein ligase ZFP91 (ZFP91), Protein C-ets-1 (ETS1), Max-binding protein MNT (MNT), myocyte-specific enhancer factor 2B (MEF2B), snRNA-activating protein complex subunit 1 (SNAPC1), lysine-specific demethylase 4B (KDM4B), transcription factor AP-4 (TFAP4), nucleolar transcription factor 1 (UBTF), bromo adjacent homology domain-containing 1 protein (BAHD1), methyl-CpG-binding domain protein 4 (MBD4), chromobox protein homolog 2 (CBX2), tumor protein 63 (TP63), transducin-like enhancer protein 3 (TLE3), forkhead box protein P1 (FOXP1), zinc finger and BTB domain-containing protein 11 (ZBTB11), interferon regulatory factor 4 (IRF4), mediator of RNA polymerase II transcription subunit 26 (MED26), cyclic AMP-dependent transcription factor ATF-7 (ATF7), zinc finger protein 644 (ZNF644), lysine-specific demethylase 5B (KDM5B), upstream stimulatory factor 2 (USF2), transcription factor 25 (TCF25), lysine-specific demethylase 4A (KDM4A), lethal (3) malignant brain tumor-like protein 2 (L3MBTL2), nRNA-activating protein complex subunit 4 (SNAPC4), lysine-specific demethylase 5 (KDM5), transcription factor COE1 (EBF1), forkhead box protein J2 (FOXJ2), nuclear factor of activated T-cells, cytoplasmic 1 (NFATC1), mRNA decay activator protein ZFP36 (ZFP36), hepatoma-derived growth factor (HDGF), ETS-related transcription factor Elf-1 (ELF1), promyelocytic leukemia protein (PML), Myb-related protein B MYBL2, mothers against decapentaplegic homolog 2 (SMAD2), chromodomain-helicase-DNA-binding protein 2 (CHD2), signal transducer and activator of transcription 1 (STAT1), Paired box protein Pax-5 (PAX5), Signal transducer and activator of transcription 2 (STAT2), pygopus homolog 2 (PYGO2), interferon regulatory factor 9 (IRF9), polycomb group RING finger protein 2 (PCGF2), and cyclic AMP-dependent transcription factor ATF-3 (ATF3).

In some embodiments, the biomarker includes one or more genes selected from the group consisting of Interleukin-23 subunit alpha (IL23A), C—C motif chemokine 2 (CCL2), and SLIT-ROBO Rho GTPase-activating protein 1 (SRGAP1).

In certain aspects, the biomarker includes a marker of T-cell activation. Activation of T-cells can promote the cytotoxic killing of a hematological cancer cells, and therefore can be beneficial in treatment of a hematological cancer. In certain embodiments, T-cell activation includes a T-cell activation associated cytokine. In specific embodiments, the T-cell activation associated cytokine comprises interleukin 2 (IL-2). In some embodiments, the biomarker is a marker of T-cell activation and the biomarker in the sample is higher than a reference level of the biomarker. For example, in certain embodiments, the reference level is the level of the biomarker prior to the administration of the compound, and a higher level of the marker of T-cell activation post administration as compared to the reference level is indicative of effectiveness of the treatment. In specific embodiments, the reference level is the level of the biomarker prior to the administration of the compound, and a higher level of IL-2 in the sample post administration as compared to the reference level is indicative of effectiveness of the treatment. However, it is understood that the biomarker in the sample need not be higher than the reference level of the biomarker. Therefore, in some embodiments, the biomarker in the sample is lower than the reference level of the biomarker. For example, in some embodiments, the reference level is the level of the biomarker post a first administration of the compound, and a lower level of the biomarker is informative for adjusting a dosage amount or frequency for treating a subject having a hematological cancer. In one embodiment, the hematological cancer is DLBCL. In another embodiment, the hematological cancer is CLL/SLL.

In other embodiments, the biomarker is a marker of exhausted T cells. Exhausted T cells differ phenotypically from functional effector T cells as they acquire the expression of inhibitory signaling pathways including the programmed cell death protein 1 (PD1) and the lymphocyte activation gene 3 protein (LAG3). Exhausted T cells show reduced differentiation, proliferation, and reduced production of effector cytokines/chemokines (e.g., GM-CSF, TNFα and IFNγ). Accordingly, in some embodiments, the biomarker includes PD1, LAG3, or a combination thereof. In further embodiments, the biomarker includes granulocyte-macrophage colony-stimulating factor (GM-CSF), tumor necrosis factor alpha (TNFα), interferon gamma (IFNγ), or a combination thereof.

In some embodiments, the biomarker is a marker of exhausted T cells and the biomarker in the sample is lower than a reference level of the biomarker. For example, in certain embodiments, the reference level is the level of the biomarker prior to the administration of the compound, and a lower level of a marker of exhausted T cells as compared to the reference level is indicative of effectiveness of the treatment. In specific embodiments, the reference level is the level of the biomarker prior to the administration of the compound, and a lower level of PD1, LAG3, or a combination thereof in the sample post administration as compared to the reference level is indicative of effectiveness of the treatment. However, it is understood that the biomarker in the sample need not be lower than the reference level of the biomarker. Therefore, in some embodiments, the biomarker in the sample is higher than the reference level of the biomarker. For example, in some embodiments, the reference level is the level of the biomarker post a first administration of the compound, and a higher level of GM-CSF, TNFα, IFNγ, or a combination thereof in the sample post administration as compared to the reference level is indicative of effectiveness of the treatment.

In some aspects, the biomarker is a marker of cytotoxicity in non-cancer cells. Neutrophils represent the first line of defense against infection as the first cellular component of the inflammatory response and a key component of innate immunity. Neutropenia blunts the inflammatory response to nascent infections, allowing bacterial multiplication and invasion. Complications from neutropenia remain the main dose-limiting toxicity of cancer chemotherapy treatment and are associated with considerable morbidity and mortality. Ex vivo maturation of neutrophils can therefore be useful in treating a hematological cancer, as well as adjusting a dosage amount or frequency for treating a subject having a hematological cancer, with a compound of Formula (I) or an enantiomer, mixture of enantiomers, tautomer, isotopolog, or pharmaceutically acceptable salt thereof. In some embodiments, the biomarker is Ikaros expression in neutrophils. In certain embodiments, the biomarker is expressed in a white blood cells. In specific embodiments, the white blood cell comprises a myeloid cell. In further embodiments, the myeloid cell comprises a neutrophil. In yet further embodiments, the biomarker comprises neutrophils having a phenotype of CD11b⁺, CD34⁻, and CD33⁻.

In some embodiments, the biomarker is a marker of cytotoxicity in non-cancer cells and the biomarker in the sample is lower than a reference level of the biomarker. For example, in certain embodiments, the reference level is the level of the biomarker post a first administration of the compound, and a lower level of the marker of cytotoxicity in neutrophils is informative for adjusting a dosage amount or frequency for treating a subject having a hematological cancer. In specific embodiments, the reference level is the level of the biomarker post a first administration of the compound, and a lower level of Ikaros and/or neutrophils having a phenotype of CD11b⁺, CD34⁻, and CD33⁻ is informative for decreasing a dosage amount or frequency for treating a subject having a hematological cancer. However, it is understood that the biomarker in the sample need not be lower than the reference level of the biomarker. Therefore, in some embodiments, the biomarker in the sample is higher than the reference level of the biomarker. For example, in some embodiments, the reference level is the level of the biomarker post a first administration of the compound, and a higher level of Ikaros and/or neutrophils having a phenotype of CD11b⁺, CD34⁻, and CD33⁻ is informative for increasing a dosage amount or frequency for treating a subject having a hematological cancer. In one embodiment, the hematological cancer is DLBCL. In another embodiment, the hematological cancer is CLL/SLL.

Various reference samples can be used for comparison of the test sample. By way of example, non-liming types of reference sample can be, for example, an untreated sample, a treated sample from an earlier point in time during the treatment regimen, a standardized reference sample, or any other sample suitable for comparison. In some embodiments, the reference biomarker level is the biomarker level in a reference sample obtained from the subject prior to administering the treatment compound to the subject, and wherein the reference sample is from the same source as the sample. In other embodiments, the reference biomarker level is the biomarker level in a reference sample obtained from a healthy subject not having the hematological cancer, and wherein the reference sample is from the same source as the sample. In yet further embodiments, the reference biomarker level is a pre-determined biomarker level.

A person skilled in the art will understand that altered biomarker levels will have different interpretations depending on the particular biomarker, as well as the reference sample that is used for comparison. As an example, the biomarker level of, for example, an apoptotic marker following treatment with a compound of Formula (I) or an enantiomer, mixture of enantiomers, tautomer, isotopolog, or pharmaceutically acceptable salt thereof, may be higher than a reference sample that is from the subject prior to administering any treatment compound to the subject. The increased level of the biomarker can indicate that the treatment is efficacious. Alternatively, the biomarker level of, for example, a CRBN-associated protein following treatment with a compound of Formula (I) or an enantiomer, mixture of enantiomers, tautomer, isotopolog, or pharmaceutically acceptable salt thereof, may be lower than a reference sample that is from the subject prior to administering any treatment compound to the subject. The decreased level of the biomarker in such a circumstance can indicate that the treatment is efficacious.

However, the same biomarker can have a different level in the same sample when it is compared to a different reference sample. By way of example, a biomarker level of, for example, a CRBN-associated protein, may be higher than a reference sample that is from the same subject, but the reference sample was obtained at an earlier point in the treatment regimen, and still indicate efficacy of the treatment because the biomarker level is still lower than a reference sample that is from the subject prior to administering any treatment compound to the subject. Accordingly, the biomarker level and the meaning of the biomarker level will depend on the context of the reference sample.

Thus, in some embodiments the biomarker in the sample is higher than the reference level of the biomarker. In other embodiments, the biomarker in the sample is lower than the reference level of the biomarker. In yet other embodiments, detection of the biomarker can indicate that the subject is responsive. In certain embodiments, an increased biomarker level relative to the reference biomarker level is indicative of the efficacy of the treatment compound in treating the hematological cancer in the subject. In other embodiments, a decreased biomarker level relative to the reference biomarker level is indicative of the efficacy of the treatment compound in treating the hematological cancer in the subject. In one embodiment, the hematological cancer is DLBCL. In another embodiment, the hematological cancer is CLL/SLL.

Detection of the biomarkers is within the skillset of a person skilled in the art. For example, in certain embodiments determining the biomarker level comprises determining the protein level of the biomarker. In other embodiments, determining the biomarker level comprises determining the mRNA level of the biomarker. In further embodiments, the determining the biomarker level comprises determining the cDNA level of the biomarker as a surrogate marker for determining the RNA level. Exemplary assays provided herein for the methods of detecting and quantifying the protein level of a biomarker, such as Aiolos, Ikaros, CRBN, c-MYC, IRF4, ZFP91, BCL2, BCL6, MCL1, IRF7, IFIT3, cleaved-Caspase-3, cleaved-Caspase-7, cleaved-PARP, BIM, DDX58, Survivin, PD1, LAG3, activated T-cell-associated cytokines, or a combination thereof, are immunoassays, such as western blot analysis, enzyme-linked immunosorbent assay (ELISA) (e.g., a sandwich ELISA), immunohistochemistry (IHC), and fluorescence-activated cell sorting (FACS). Exemplary assays provided herein for the methods of detecting and quantifying the RNA level of a biomarker, such as Aiolos, Ikaros, ZFP91, CRBN, c-MYC, IRF4, activated T-cell-associated cytokines, CD142 (tissue factor), CD62E (E-selectin), interleukin-8 (IL8), interleukin-2 (IL2), interleukin-6 (IL-6), interleukin-17A (IL17A), interleukin-17F (IL17F), collagen-I, collagen-III, PAI-I, interleukin-10 (IL-10), CD69, immunoglobulin (IgG)tumor necrosis factor alpha (TNFα), or a combination thereof, are reverse transcription polymerase chain reaction (RT-PCR), e.g., quantitative RT-PCR (qRT-PCR), and RNA-Seq.

It is understood that the techniques described above for the detection of the biomarkers are non-limiting and that a person skilled in the art can use any of the known techniques to detect and measure the biomarkers provided herein. Accordingly, the practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999); Glover, ed., DNA Cloning, Volumes I and II (1985); Mullis et al., Cold Spring Harbor Symp. Quant. Biol. 1987, 51:263-273; PCR Technology (Stockton Press, NY, Erlich, ed., 1989); Gait, ed., Oligonucleotide Synthesis (1984); Hames & Higgins, eds., Nucleic Acid Hybridization (1984); Hames & Higgins, eds., Transcription and Translation (1984); Freshney, ed., Animal Cell Culture: Immobilized Cells and Enzymes (IRL Press, 1986); Immunochemical Methods in Cell and Molecular Biology (Academic Press, London); Scopes, Protein Purification: Principles and Practice (Springer Verlag, N.Y., 2d ed. 1987); and Weir & Blackwell, eds., Handbook of Experimental Immunology, Volumes I-IV (1986).

5.4 Methods of Treatment, and/or Management

Provided herein are methods of treating, and/or managing a hematological cancer, comprising administering a therapeutically effective amount of a compound of Formula (I) or an enantiomer, mixture of enantiomers, tautomer, isotopolog, or pharmaceutically acceptable salt thereof.

In one aspect, provided herein is a method of selectively treating a hematological cancer in a subject having a hematological cancer, that includes (a) obtaining a sample from the subject having a hematological cancer; (b) determining a biomarker level in the sample; (c) diagnosing the subject as being likely to be responsive to a treatment compound if: (i) the biomarker level in the sample is detectable; or (ii) the biomarker level is an altered level relative to a reference level of the biomarker; and (d) administering a therapeutically effective amount of the treatment compound to the subject diagnosed as being likely to be responsive to the treatment compound; wherein the treatment compound is a compound of Formula (I), or an enantiomer, mixture of enantiomers, tautomer, isotopolog, or pharmaceutically acceptable salt thereof. In some embodiments, the compound of Formula (I) comprises a compound selected from the group consisting of (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof; (R)-2-(2,6-Dioxopiperidin-3-yl)-4-((2-fluoro-4-((3-morpholinoazetidin-1-yl)methyl)benzyl)amino)isoindoline-1,3-dione (Compound 2), or a tautomer, isotopolog, or pharmaceutically acceptable salt thereof; or 2-(2,6-Dioxopiperidin-3-yl)-4-((2-fluoro-4-((3-morpholinoazetidin-1-yl)methyl)benzyl)amino)isoindoline-1,3-dione (Compound 3), or an enantiomer, mixture of enantiomers, tautomer, isotopolog, or pharmaceutically acceptable salt thereof. In certain embodiments, the treatment compound of Formula (I) is (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof. In some embodiments, the hematological cancer comprises non-Hodgkin's lymphoma. In specific embodiments, the non-Hodgkin's lymphoma is diffuse large B-cell lymphoma (DLBCL). In yet further embodiments, the DLBCL is relapsed, refractory, or resistant to conventional therapy. In other embodiments, the hematological cancer is CLL/SLL. In yet further embodiments, the CLL/SLL is relapsed, refractory, or resistant to conventional therapy.

Also provided herein is a method of treating a hematological cancer, that includes (a) obtaining a first sample from a subject having a hematological cancer; (b) determining a biomarker level in the first sample; (c) administering a therapeutically effective amount of a treatment compound to the subject; (d) obtaining at least one additional sample from the subject after the treatment; and (e) determining the biomarker level in the at least one additional sample; and if the biomarker level in the at least one additional sample is at or near the biomarker level of the first sample, then administering another therapeutically effective amount of the treatment compound to the subject, wherein the treatment compound is a compound of Formula (I), or an enantiomer, mixture of enantiomers, tautomer, isotopolog, or pharmaceutically acceptable salt thereof. In some embodiments, the compound of Formula (I) comprises a compound selected from the group consisting of (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof (R)-2-(2,6-Dioxopiperidin-3-yl)-4-((2-fluoro-4-((3-morpholinoazetidin-1-yl)methyl)benzyl)amino)isoindoline-1,3-dione (Compound 2), or a tautomer, isotopolog, or pharmaceutically acceptable salt thereof; or 2-(2,6-Dioxopiperidin-3-yl)-4-((2-fluoro-4-((3-morpholinoazetidin-1-yl)methyl)benzyl)amino)isoindoline-1,3-dione (Compound 3), or an enantiomer, mixture of enantiomers, tautomer, isotopolog, or pharmaceutically acceptable salt thereof. In certain embodiments, the treatment compound of Formula (I) is (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof. In some embodiments, the hematological cancer comprises non-Hodgkin's lymphoma. In specific embodiments, the non-Hodgkin's lymphoma is DLBCL. In yet further embodiments, the DLBCL is relapsed, refractory, or resistant to conventional therapy. In other embodiments, the hematological cancer is CLL/SLL. In yet further embodiments, the CLL/SLL is relapsed, refractory, or resistant to conventional therapy.

In addition, provided herein is a method of monitoring the efficacy of a treatment compound in treating a hematological cancer in a subject, that includes: (a) administering a treatment compound to a subject; (b) obtaining a sample from the subject; (c) determining a biomarker level in the sample; and (d) comparing the biomarker level in the sample with a reference biomarker level, wherein an altered biomarker level is indicative of the efficacy of the treatment compound in treating a hematological cancer in the subject; wherein the treatment compound is a compound of Formula (I), or an enantiomer, mixture of enantiomers, tautomer, isotopolog, or pharmaceutically acceptable salt thereof. In some embodiments, the compound of Formula (I) comprises a compound selected from the group consisting of (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof (R)-2-(2,6-Dioxopiperidin-3-yl)-4-((2-fluoro-4-((3-morpholinoazetidin-1-yl)methyl)benzyl)amino)isoindoline-1,3-dione (Compound 2), or a tautomer, isotopolog, or pharmaceutically acceptable salt thereof; or 2-(2,6-Dioxopiperidin-3-yl)-4-((2-fluoro-4-((3-morpholinoazetidin-1-yl)methyl)benzyl)amino)isoindoline-1,3-dione (Compound 3), or an enantiomer, mixture of enantiomers, tautomer, isotopolog, or pharmaceutically acceptable salt thereof. In certain embodiments, the treatment compound of Formula (I) is (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof. In some embodiments, the hematological cancer comprises non-Hodgkin's lymphoma. In specific embodiments, the non-Hodgkin's lymphoma is DLBCL. In yet further embodiments, the DLBCL is relapsed, refractory, or resistant to conventional therapy. In other embodiments, the hematological cancer is CLL/SLL. In yet further embodiments, the CLL/SLL is relapsed, refractory, or resistant to conventional therapy.

Provided herein is also a method of identifying a subject having a hematological cancer who is likely to be responsive to a treatment compound or predicting the responsiveness of a subject having or suspected of having a hematological cancer to a treatment compound. In some embodiments, the method of identifying a subject having a hematological cancer who is likely to be responsive to a treatment compound or predicting the responsiveness of a subject having or suspected of having a hematological cancer to a treatment compound includes (a) obtaining a sample from the subject; (b) administering the treatment compound to the sample; (c) determining a biomarker level in the sample; and (d) diagnosing the subject as being likely to be responsive to the treatment compound if the biomarker level in the sample is an altered level relative to a reference biomarker level; wherein the treatment compound is a compound of Formula (I), or an enantiomer, mixture of enantiomers, tautomer, isotopolog, or pharmaceutically acceptable salt thereof. In some embodiments, the compound of Formula (I) comprises a compound selected from the group consisting of (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof; (R)-2-(2,6-Dioxopiperidin-3-yl)-4-((2-fluoro-4-((3-morpholinoazetidin-1-yl)methyl)benzyl)amino)isoindoline-1,3-dione (Compound 2), or a tautomer, isotopolog, or pharmaceutically acceptable salt thereof; or 2-(2,6-Dioxopiperidin-3-yl)-4-((2-fluoro-4-((3-morpholinoazetidin-1-yl)methyl)benzyl)amino)isoindoline-1,3-dione (Compound 3), or an enantiomer, mixture of enantiomers, tautomer, isotopolog, or pharmaceutically acceptable salt thereof. In certain embodiments, the treatment compound of Formula (I) is (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof. In some embodiments, the hematological cancer comprises non-Hodgkin's lymphoma. In specific embodiments, the non-Hodgkin's lymphoma is diffuse large B-cell lymphoma (DLBCL). In some embodiments, the hematological cancer comprises non-Hodgkin's lymphoma. In specific embodiments, the non-Hodgkin's lymphoma is DLBCL. In yet further embodiments, the DLBCL is relapsed, refractory, or resistant to conventional therapy. In other embodiments, the hematological cancer is CLL/SLL. In yet further embodiments, the CLL/SLL is relapsed, refractory, or resistant to conventional therapy.

In other embodiments, the method of identifying a subject having a hematological cancer who is likely to be responsive to a treatment compound or predicting the responsiveness of a subject having or suspected of having a hematological cancer to a treatment compound includes (a) administering a treatment compound to a subject; (b) obtaining a sample from the subject; (c) determining a biomarker level in the sample; and (d) diagnosing the subject as being likely to be responsive to the treatment compound if the biomarker level in the sample is an altered level relative to a reference biomarker level; wherein the treatment compound is a compound of Formula (I), or an enantiomer, mixture of enantiomers, tautomer, isotopolog, or pharmaceutically acceptable salt thereof. In some embodiments, the compound of Formula (I) comprises a compound selected from the group consisting of (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof; (R)-2-(2,6-Dioxopiperidin-3-yl)-4-((2-fluoro-4-((3-morpholinoazetidin-1-yl)methyl)benzyl)amino)isoindoline-1,3-dione (Compound 2), or a tautomer, isotopolog, or pharmaceutically acceptable salt thereof; or 2-(2,6-Dioxopiperidin-3-yl)-4-((2-fluoro-4-((3-morpholinoazetidin-1-yl)methyl)benzyl)amino)isoindoline-1,3-dione (Compound 3), or an enantiomer, mixture of enantiomers, tautomer, isotopolog, or pharmaceutically acceptable salt thereof. In certain embodiments, the treatment compound of Formula (I) is (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof. In some embodiments, the hematological cancer comprises non-Hodgkin's lymphoma. In specific embodiments, the non-Hodgkin's lymphoma is DLBCL. In yet further embodiments, the DLBCL is relapsed, refractory, or resistant to conventional therapy. In other embodiments, the hematological cancer is CLL/SLL. In yet further embodiments, the CLL/SLL is relapsed, refractory, or resistant to conventional therapy.

In yet further embodiments, the method of identifying a subject having a hematological cancer who is likely to be responsive to a treatment compound or predicting the responsiveness of a subject having or suspected of having a hematological cancer to a treatment compound includes (a) obtaining a sample from the subject; (b) determining a biomarker level in the sample; (c) diagnosing the subject as being likely to be responsive to the treatment compound if: (i) the biomarker level in the sample is detectable; or (ii) the biomarker level in the sample is an altered level relative to a reference biomarker level; and wherein the treatment compound is a compound of Formula (I), or an enantiomer, mixture of enantiomers, tautomer, isotopolog, or pharmaceutically acceptable salt thereof. In some embodiments, the compound of Formula (I) comprises a compound selected from the group consisting of (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof; (R)-2-(2,6-Dioxopiperidin-3-yl)-4-((2-fluoro-4-((3-morpholinoazetidin-1-yl)methyl)benzyl)amino)isoindoline-1,3-dione (Compound 2), or a tautomer, isotopolog, or pharmaceutically acceptable salt thereof; or 2-(2,6-Dioxopiperidin-3-yl)-4-((2-fluoro-4-((3-morpholinoazetidin-1-yl)methyl)benzyl)amino)isoindoline-1,3-dione (Compound 3), or an enantiomer, mixture of enantiomers, tautomer, isotopolog, or pharmaceutically acceptable salt thereof. In certain embodiments, the treatment compound of Formula (I) is (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof. In some embodiments, the hematological cancer comprises non-Hodgkin's lymphoma. In specific embodiments, the non-Hodgkin's lymphoma is DLBCL. In yet further embodiments, the DLBCL is relapsed, refractory, or resistant to conventional therapy. In other embodiments, the hematological cancer is CLL/SLL. In yet further embodiments, the CLL/SLL is relapsed, refractory, or resistant to conventional therapy.

As described in Section 5.3, various biomarkers can be used in the methods provided herein. In some embodiments, the detection or altered level of the biomarkers provided herein can be used in the methods to identify a subject having a hematological cancer who is likely to be responsive to a treatment compound. By way of example, the detection of, for example, CRBN, can indicate that a subject having a hematological cancer is likely to be responsive. Another exemplary biomarker, such as for example a CRBN-associated protein (e.g., IKAROS, AIOLOS, ZFP91) can decrease relative to an untreated sample in response to treatment with a compound of Formula (I) or an enantiomer, mixture of enantiomers, tautomer, isotopolog, or pharmaceutically acceptable salt thereof, and be used in, for example, a method of identifying a subject having a hematological cancer who is likely to be responsive to a treatment compound or predicting the responsiveness of a subject having or suspected of having a hematological cancer to a treatment compound. In other embodiments, the detection or altered level of the biomarkers provided herein can be used in, for example, the methods of treating a hematological cancer or monitoring the efficacy of a treatment compound in treating a hematological cancer in a subject having a hematological cancer. As an example, a treatment compound of Formula (I) or an enantiomer, mixture of enantiomers, tautomer, isotopolog, or pharmaceutically acceptable salt thereof can be administered to a subject and the level of a biomarker in a sample from the treated subject can be compared to a reference sample from the same subject prior to any treatment. An increase in the biomarker level of, for example, an apoptotic protein, can indicate that the treatment is efficacious and guide the practitioner in the treatment of the subject. It is understood that the examples described above are exemplary and are not inclusive of the biomarkers that can be with the methods provided herein.

### 5.5 Methods of Adjusting a Dosage Amount or Frequency

Provided herein is also a method of adjusting a dosage amount or frequency for treating a subject having a hematological cancer with a treatment compound that includes: (a) administering a dosage of a treatment compound to a subject; (b) obtaining one of more samples from the subject at different time points; and (c) monitoring a biomarker level in the one or more samples, and (d) adjusting the dosage for subsequent administration of the treatment compound to the subject based upon an altered level of the biomarker in a reference sample, wherein the treatment compound is a compound of Formula (I): or an enantiomer, mixture of enantiomers, tautomer, isotopolog, or pharmaceutically acceptable salt thereof. In certain embodiments, the cycling schedule is determined based on the detection of a biomarker level. In some embodiments, the compound of Formula (I) comprises a compound selected from the group consisting of (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof; (R)-2-(2,6-Dioxopiperidin-3-yl)-4-((2-fluoro-4-((3-morpholinoazetidin-1-yl)methyl)benzyl)amino)isoindoline-1,3-dione (Compound 2), or a tautomer, isotopolog, or pharmaceutically acceptable salt thereof; or 2-(2,6-Dioxopiperidin-3-yl)-4-((2-fluoro-4-((3-morpholinoazetidin-1-yl)methyl)benzyl)amino)isoindoline-1,3-dione (Compound 3), or an enantiomer, mixture of enantiomers, tautomer, isotopolog, or pharmaceutically acceptable salt thereof. In certain embodiments, the treatment compound of Formula (I) is (S)-2-(2,6-Dioxopiperidin-3-yl)-4-((2-fluoro-4-((3-morpholinoazetidin-1-yl)methyl)benzyl)amino)isoindoline-1,3-dione (Compound 1), or a tautomer, isotopolog, or pharmaceutically acceptable salt thereof. In some embodiments, the hematological cancer comprises non-Hodgkin's lymphoma. In specific embodiments, the non-Hodgkin's lymphoma is diffuse large B-cell lymphoma (DLBCL). In yet further embodiments, the DLBCL is relapsed, refractory, or resistant to conventional therapy. In other embodiments, the hematological cancer is CLL/SLL. In yet further embodiments, the CLL/SLL is relapsed, refractory, or resistant to conventional therapy.

Various biomarkers can be used to determine whether the dosage amount of frequency of treatment needs adjusting. In certain embodiments, the biomarker used in the method of adjusting a dosage amount of frequency can be selected from the group consisting of mature neutrophils, and Ikaros protein levels in neutrophils. For example, ex vivo maturation of neutrophils can be used as a biomarker to evaluate, for example, myeloid toxicity. In certain embodiments, ex vivo cultures of bone marrow CD34+ cells can be exposed to different dosing schedules of treatment with a compound of Formula (I) or an enantiomer, mixture of enantiomers, tautomer, isotopolog, or pharmaceutically acceptable salt thereof, and myeloid differentiation can be induced, for example, by using stem cell factor (SCF), FMS-related tyrosine kinase 3 ligand (FLT3-L), and granulocyte colony stimulating factor (G-CSF) to culture media. Cell differentiation in the presence or absence of the compound of Formula (I) or an enantiomer, mixture of enantiomers, tautomer, isotopolog, or pharmaceutically acceptable salt thereof can be evaluated at set time points in different populations of cells (e.g., hematopoietic stem cells (HSC, CD34+/CD33−/CD11b−); Stage I cells (CD34+/CD33+/CD11b−); Stage II cells (CD134−/CD33+/CD11b−); Stage III cells (CD34−/CD33+/CD11b+) and Stage IV cells (CD34−/CD33−/CD11b+) cells). In certain embodiments, the recovery of the mature neutrophils (Stave IV cells) after exposure to a compound of Formula (I) or an enantiomer, mixture of enantiomers, tautomer, isotopolog, or pharmaceutically acceptable salt thereof can serve as a biomarker.

In other embodiments, Ikaros protein levels in neutrophils can be a biomarker for determining the dosing schedule and/or cytotoxicity. As provided herein, Ikaros levels were found to be reduced in neutrophils during exposure with a compound of Formula (I) or an enantiomer, mixture of enantiomers, tautomer, isotopolog, or pharmaceutically acceptable salt thereof, and recovered following drug withdrawal in a concentration-dependent manner. Full recovery of maturation of late-stage neutrophils was immediately preceded by recovery of Ikaros levels. Thus, in some embodiments, Ikaros degradation and/or Ikaros recovery in neutrophils can be a biomarker for response to a compound of Formula (I) or an enantiomer, mixture of enantiomers, tautomer, isotopolog, or pharmaceutically acceptable salt thereof, as well as a biomarker for cytotoxicity. In addition, Ikaros protein levels in neutrophils and/or maturation of neutrophils can be a biomarker for determining the cycling schedule. By way of example, a recovery of Ikaros protein levels after administration of a compound of Formula (I) or an enantiomer, mixture of enantiomers, tautomer, isotopolog, or pharmaceutically acceptable salt thereof to at least about 30% or more, about 40% or more, about 50% or more, about 60% or more, about 70% or more can be used as a biomarker before a subsequent administration of a compound of Formula (I) or an enantiomer, mixture of enantiomers, tautomer, isotopolog, or pharmaceutically acceptable salt thereof is performed.

It is understood that the biomarkers described above for use in a method of adjusting a dosage amount of frequency for treating a subject having a hematological cancer with a treatment compound are non-limiting and that other biomarkers which indicate the amount, stage, and/or viability of mature neutrophils can be useful as biomarkers.

### 5.6 Pharmaceutical Compositions and Routes of Administration

As provided herein, the compound of Formula (I) or a pharmaceutically acceptable salt, solvate, hydrate, stereoisomer, tautomer or racemic mixtures thereof, such as any of the compounds described in Section 5.2, can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfate, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds in the pharmaceutical composition may be at a level that will exercise the desired effect; about 0.001 mg/kg of a subject's body weight to about 1 mg/kg of a subject's body weight in unit dosage for both oral and parenteral administration.

A compound provided herein can be administered orally. In one embodiment, when administered orally, a compound provided herein is administered with a meal and water. In another embodiment, the compound provided herein is dispersed in water or juice (e.g., apple juice or orange juice) and administered orally as a solution or a suspension.

The compound provided herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a compound provided herein without an additional carrier, excipient or vehicle. In another embodiment, provided herein are compositions comprising an effective amount of a compound provided herein and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts. In general, all of the compositions are prepared according to known methods in pharmaceutical chemistry. Capsules can be prepared by mixing a compound provided herein with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound provided herein as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound provided herein can be delayed or prolonged by proper formulation. For example, a slowly soluble pellet of the compound provided herein can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound provided herein in oily or emulsified vehicles that allow it to disperse slowly in the serum.

The methods provided herein encompass treating a patient regardless of patient's age. In some embodiments, the subject is 18 years or older. In other embodiments, the subject is more than 18, 25, 35, 40, 45, 50, 55, 60, 65, or 70 years old. In other embodiments, the subject is less than 65 years old. In other embodiments, the subject is more than 65 years old.

Depending on the state of the disease to be treated and the subject's condition, Compound 1, Compound 2 or Compound 3 provided herein, or an enantiomer, mixture of enantiomers, tautomer, isotopolog, or pharmaceutically acceptable salt thereof, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. Compound 1, Compound 2 or Compound 3 provided herein, or an enantiomer, mixture of enantiomers, tautomer, isotopolog, or pharmaceutically acceptable salt thereof, may be formulated, alone or together, in suitable dosage unit with pharmaceutically acceptable excipients, carriers, adjuvants and vehicles, appropriate for each route of administration.

In one embodiment, Compound 1, Compound 2 or Compound 3 provided herein, or an enantiomer, mixture of enantiomers, tautomer, isotopolog, or pharmaceutically acceptable salt thereof, is administered orally. In another embodiment, the compound of Compound 1, Compound 2 or Compound 3 provided herein, or an enantiomer, mixture of enantiomers, tautomer, isotopolog, or pharmaceutically acceptable salt thereof, is administered parenterally. In yet another embodiment, the compound of Compound 1, Compound 2 or Compound 3 provided herein, or an enantiomer, mixture of enantiomers, tautomer, isotopolog, or pharmaceutically acceptable salt thereof, is administered intravenously.

Compound 1, Compound 2 or Compound 3 provided herein, or an enantiomer, mixture of enantiomers, tautomer, isotopolog, or pharmaceutically acceptable salt thereof, can be delivered as a single dose such as, e.g., a single bolus injection, or oral capsules, tablets or pills; or over time, such as, e.g., continuous infusion over time or divided bolus doses over time. The compounds as described herein can be administered repeatedly if necessary, for example, until the patient experiences stable disease or regression, or until the patient experiences disease progression or unacceptable toxicity.

Compound 1, Compound 2 or Compound 3 provided herein, or an enantiomer, mixture of enantiomers, tautomer, isotopolog, or pharmaceutically acceptable salt thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as Compound 1, Compound 2 or Compound 3 provided herein, or an enantiomer, mixture of enantiomers, tautomer, isotopolog, or pharmaceutically acceptable salt thereof, is administered once or more than once each day, for example, for a period of time. The term “continuous” is intended to mean that a therapeutic compound, such as Compound 1, Compound 2 or Compound 3 provided herein, or an enantiomer, mixture of enantiomers, tautomer, isotopolog, or pharmaceutically acceptable salt thereof, is administered daily for an uninterrupted period of at least 7 days to 52 weeks. The term “intermittent” or “intermittently” as used herein is intended to mean stopping and starting at either regular or irregular intervals. For example, intermittent administration of Compound 1, Compound 2 or Compound 3 provided herein, or an enantiomer, mixture of enantiomers, tautomer, isotopolog, or pharmaceutically acceptable salt thereof, is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as Compound 1, Compound 2 or Compound 3 provided herein, or an enantiomer, mixture of enantiomers, tautomer, isotopolog, or pharmaceutically acceptable salt thereof, is administered daily or continuously but with a rest period.

In some embodiments, the frequency of administration is in the range of about a daily dose to about a monthly dose. In one embodiment, administration is once a day, twice a day, three times a day, four times a day, once every other day, twice a week, once every week, once every two weeks, once every three weeks, or once every four weeks. In one embodiment, Compound 1, Compound 2 or Compound 3 provided herein, or an enantiomer, mixture of enantiomers, tautomer, isotopolog, or pharmaceutically acceptable salt thereof, is administered once a day. In another embodiment, Compound 1, Compound 2 or Compound 3 provided herein, or an enantiomer, mixture of enantiomers, tautomer, isotopolog, or pharmaceutically acceptable salt thereof, is administered twice a day. In yet another embodiment, Compound 1, Compound 2 or Compound 3 provided herein, or an enantiomer, mixture of enantiomers, tautomer, isotopolog, or pharmaceutically acceptable salt thereof, is administered three times a day. In still another embodiment, Compound 1, Compound 2 or Compound 3 provided herein, or an enantiomer, mixture of enantiomers, tautomer, isotopolog, or pharmaceutically acceptable salt thereof, is administered four times a day.

In one embodiment, the methods provided herein include an administration of a therapeutically effective amount of Compound 1, Compound 2 or Compound 3 in one or more 7-day treatment cycles. In another embodiment, the methods provided herein include an administration of a therapeutically effective amount of Compound 1, Compound 2 or Compound 3 on days 1 to 5 of a 7-day cycle. In one embodiment, Compound 1, Compound 2 or Compound 3 is administered once daily for 5 days followed by 2 days of rest. In another embodiment, the methods provided herein include an administration of a therapeutically effective amount of Compound 1, Compound 2 or Compound 3 on days 1 to 5, days 8 to 12, days 15 to 19, and days 22 to 26 of a 28-day cycle.

In one embodiment, the hematological cancer is chronic lymphoid leukemia (CLL) and the treatment includes an administration of a therapeutically effective amount of a second active agent in one or more treatment cycles. In one embodiment, the second active agent is administered twice every 7 days. In one embodiment, the second active agent is administered once every week. In one embodiment, the second active agent is administered once every 4 weeks. In one embodiment, the second active agent is administered at days 1, 2, 8, and 15 of the first 28-day cycle, and administered at day 1 of the second to the sixth 28-day cycles.

Any treatment cycle described herein can be repeated for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more cycles. In certain instances, the treatment cycle as described herein includes from 1 to about 24 cycles, from about 2 to about 16 cycles, or from about 2 to about 4 cycles. In certain instances a treatment cycle as described herein includes from 1 to about 4 cycles. In some embodiments, a therapeutically effective amount of Compound 1, Compound 2 or Compound 3, and/or a second active agent is administered for 1 to 24 cycles of 28 days (e.g., about 2 year). In certain instances, the cycling therapy is not limited to the number of cycles, and the therapy is continued until disease progression. Cycles can in certain instances include varying the duration of administration periods and/or rest periods described herein.

### 5.7 Second Active Agents

A compound of Formula (I) or an enantiomer, mixture of enantiomers, tautomer, isotopolog, or pharmaceutically acceptable salt thereof can be combined with other pharmacologically active compounds (“second active agents”) in methods and compositions provided herein. Certain combinations may work synergistically in the treatment of particular types of diseases or disorders, and conditions and symptoms associated with such diseases or disorders. A compound of Formula (I) or an enantiomer, mixture of enantiomers, tautomer, isotopolog, or pharmaceutically acceptable salt thereof can also work to alleviate adverse effects associated with certain second active agents, and vice versa.

One or more second active ingredients or agents can be used in the methods and compositions provided herein. Second active agents can be large molecules (e.g., proteins) or small molecules (e.g., synthetic inorganic, organometallic, or organic molecules). Various agents can be used, such as those described in U.S. patent application Ser. No. 16/390,815 or U.S. Provisional Application entitled, “SUBSTITUTED 4-AMINOISOINDOLINE-1,3-DIONE COMPOUNDS AND SECOND ACTIVE AGENTS FOR COMBINED USE,” filed on even date herewith (Attorney Docket No. 14247-390-888), each of which is incorporated herein by reference in their entirety. Exemplary second active agents include, but are not limited to, an HDAC inhibitor (e.g., panobinostat, romidepsin, vorinostat, or citarinostat), a BCL2 inhibitor (e.g., venetoclax), a BTK inhibitor (e.g., ibrutinib or acalabrutinib), an mTOR inhibitor (e.g., everolimus), a PI3K inhibitor (e.g., idelalisib), a PKCβ inhibitor (e.g., enzastaurin), a SYK inhibitor (e.g., fostamatinib), a JAK2 inhibitor (e.g., fedratinib, pacritinib, ruxolitinib, baricitinib, gandotinib, lestaurtinib, or momelotinib), an Aurora A kinase inhibitor (e.g., alisertib), an EZH2 inhibitor (e.g., tazemetostat, GSK126, CPI-1205, 3-deazaneplanocin A, EPZ005687, EI1, UNC1999, or sinefungin), a BET inhibitor (e.g., birabresib or 4-[2-(cyclopropylmethoxy)-5-(methanesulfonyl)phenyl]-2-methylisoquinolin-1(2H)-one), a hypomethylating agent (e.g., 5-azacytidine or decitabine), a chemotherapy (e.g., bendamustine, doxorubicin, etoposide, methotrexate, cytarabine, vincristine, ifosfamide, melphalan, oxaliplatin, or dexamethasone), or an epigenetic compound (e.g., a DOT1L inhibitor such as pinometostat, a HAT inhibitor such as C646, a WDR5 inhibitor such as OICR-9429, a DNMT1 selective inhibitor such as GSK3484862, a LSD-1 inhibitor such as Compound C or seclidemstat, a G9A inhibitor such as UNC0631, a PRMT5 inhibitor such as GSK3326595, a BRD inhibitor (e.g., a BRD9/7 inhibitor such as LP99), a SUV420H1/H2 inhibitor such as A-196, or a CARM1 inhibitor such as EZM2302).

In some embodiments of the methods described herein the methods further include administration of one or more of rituximab, cyclophosphamide, doxorubicin, vincristine, prednisone, etoposide, Bendamustine (Treanda), lenalidomide, or gemcitabine. In some embodiments of the methods described herein, the treatment further includes treatment with one or more of stem cell transplant, Bendamustine (Treanda) plus rituximab, rituximab, lenalidomide plus rituximab, or gemcitabine-based combinations. In certain embodiments, the second active agent is rituximab, as provided in U.S. Provisional Application 62/833,432.

In one embodiment, the second active agent used in the methods provided herein is a histone deacetylase (HDAC) inhibitor. In one embodiment, the HDAC inhibitor is panobinostat, romidepsin, vorinostat, or citarinostat, or a stereoisomer, mixture of stereoisomers, tautomer, isotopolog, or pharmaceutically acceptable salt thereof.

In one embodiment, the HDAC inhibitor is panobinostat, or a tautomer, isotopolog, or pharmaceutically acceptable salt thereof. In one embodiment, the HDAC inhibitor is panobinostat. In one embodiment, the HDAC inhibitor is a pharmaceutically acceptable salt of panobinostat. In one embodiment, the HDAC inhibitor is panobinostat lactate. In one embodiment, the HDAC inhibitor is a mono-lactate salt of panobinostat. Panobinostat has a chemical name of (2E)-N-hydroxy-3-[4-({[2-(2-methyl-1H-indol-3-yl)ethyl]amino}methyl)phenyl]acrylamide, and has the structure:

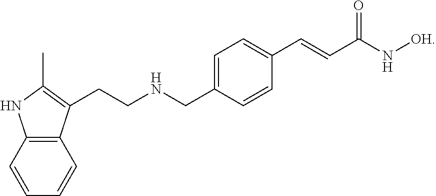 [SMILES: Cc1[nH]c2ccccc2c1CCNCc1ccc(/C=C/C(=O)NO)cc1]

In one embodiment, the HDAC inhibitor is romidepsin, or a stereoisomer, mixture of stereoisomers, tautomer, isotopolog, or pharmaceutically acceptable salt thereof. In one embodiment, the HDAC inhibitor is romidepsin. Romidepsin has a chemical name of (1S,4S,7Z,10S,16E,21R)-7-ethylidene-4,21-bis(1-methylethyl)-2-oxa-12,13-dithia-5,8,20,23-tetraazabicyclo[8.7.6]tricos-16-ene-3,6,9,19,22-pentone, and has the structure:

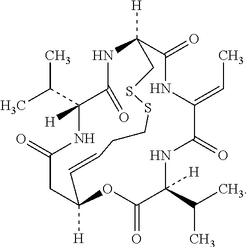 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

In one embodiment, the HDAC inhibitor is vorinostat, or a tautomer, isotopolog, or pharmaceutically acceptable salt thereof. In one embodiment, the HDAC inhibitor is vorinostat. Vorinostat has a chemical name of N-hydroxy-N′-phenyloctanediamide, and has the structure:

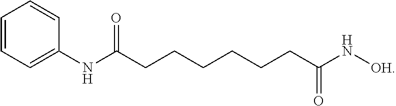 [SMILES: O=C(CCCCCCC(=O)Nc1ccccc1)NO]

In one embodiment, the HDAC inhibitor is a HDAC6 inhibitor. In one embodiment, the HDAC6 inhibitor is citarinostat, or a tautomer, isotopolog, or pharmaceutically acceptable salt thereof. In one embodiment, the HDAC6 inhibitor is citarinostat. Citarinostat (also known as ACY-241) has a chemical name of 2-((2-chlorophenyl)(phenyl)amino)-N-(7-(hydroxyamino)-7-oxoheptyl)pyrimidine-5-carboxamide, and has the structure:

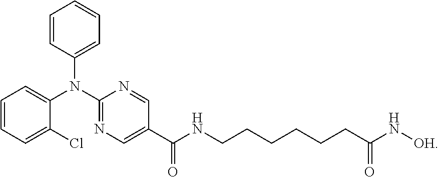 [SMILES: O=C(CCCCCCNC(=O)c1cnc(N(c2ccccc2)c2ccccc2Cl)nc1)NO]

In one embodiment, the second active agent used in the methods provided herein is a B-cell lymphoma 2 (BCL2) inhibitor. In one embodiment, the BCL2 inhibitor is venetoclax, or a tautomer, isotopolog, or pharmaceutically acceptable salt thereof. In one embodiment, the BCL2 inhibitor is venetoclax. Venetoclax has a chemical name of 4-(4-{[2-(4-chlorophenyl)-4,4-dimethylcyclohex-1-en-1-yl]methyl}piperazin-1-yl)-N-({3-nitro-4-[(tetrahydro-2H-pyran-4ylmethyl)amino]phenyl}sulfonyl)-2-(1H-pyrrolo[2,3-b]pyridin-5-yloxy)benzamide), and has the structure:

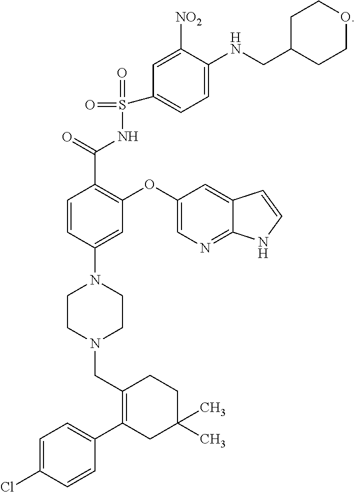 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

In one embodiment, the second active agent used in the methods provided herein is a Bruton's tyrosine kinase (BTK) inhibitor. In one embodiment, the BTK inhibitor is ibrutinib, or acalabrutinib, or a stereoisomer, mixture of stereoisomers, tautomer, isotopolog, or pharmaceutically acceptable salt thereof.

In one embodiment, the BTK inhibitor is ibrutinib, or a stereoisomer, mixture of stereoisomers, tautomer, isotopolog, or pharmaceutically acceptable salt thereof. In one embodiment, the BTK inhibitor is ibrutinib. Ibrutinib has a chemical name of 1-[(3R)-3-[4-amino-3-(4-phenoxyphenyl)-1Hpyrazolo[3,4-d]pyrimidin-1-yl]-1-piperidinyl]-2-propen-1-one, and has the structure:

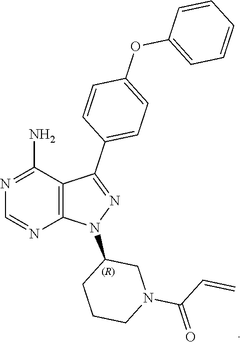

In one embodiment, the BTK inhibitor is acalabrutinib, or a stereoisomer, mixture of stereoisomers, tautomer, isotopolog, or pharmaceutically acceptable salt thereof. In one embodiment, the BTK inhibitor is acalabrutinib. Acalabrutinib has a chemical name of (S)-4-(8-amino-3-(1-(but-2-ynoyl)pyrrolidin-2-yl)imidazo[1,5-a]pyrazin-1-yl)-N-(pyridin-2-yl)benzamide, and has the structure:

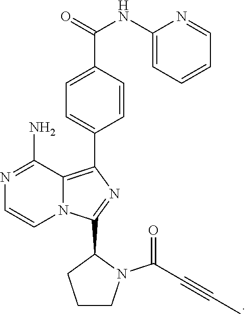 [SMILES: CC#CC(=O)N1CCC[C@H]1c1nc(-c2ccc(C(=O)Nc3ccccn3)cc2)c2c(N)nccn12]

In one embodiment, the second active agent used in the methods provided herein is a mammalian target of rapamycin (mTOR) inhibitor. In one embodiment, the mTOR inhibitor is rapamycin or an analog thereof (also termed rapalog). In one embodiment, the mTOR inhibitor is everolimus, or a stereoisomer, mixture of stereoisomers, tautomer, isotopolog, or pharmaceutically acceptable salt thereof. In one embodiment, the mTOR inhibitor is everolimus. Everolimus has a chemical name of 40-O-(2-hydroxyethyl)-rapamycin, and has the structure:

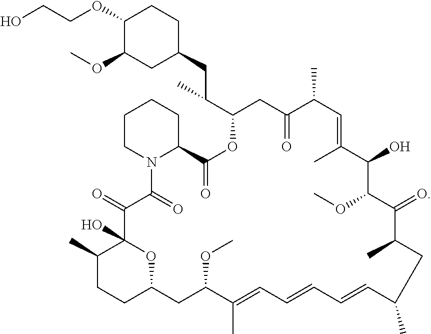 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

In one embodiment, the second active agent used in the methods provided herein is a phosphoinositide 3-kinase (PI3K) inhibitor. In one embodiment, the PI3K inhibitor is idelalisib, or a stereoisomer, mixture of stereoisomers, tautomer, isotopolog, or pharmaceutically acceptable salt thereof. In one embodiment, the PI3K inhibitor is idelalisib. Idelalisib has a chemical name of 5-fluoro-3-phenyl-2-[(1S)-1-(9H-purin-6ylamino)propyl]quinazolin-4(3H)-one, and has the structure:

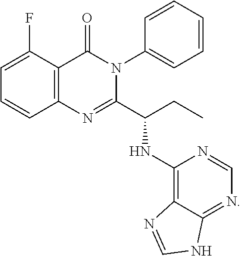 [SMILES: CC[C@H](Nc1ncnc2[nH]cnc12)c1nc2cccc(F)c2c(=O)n1-c1ccccc1]

In one embodiment, the second active agent used in the methods provided herein is a protein kinase C beta (PKCβ or PKC-β) inhibitor. In one embodiment, the PKCβ inhibitor is enzastaurin, or a stereoisomer, mixture of stereoisomers, tautomer, isotopolog, or pharmaceutically acceptable salt thereof. In one embodiment, the PKCβ inhibitor is enzastaurin. In one embodiment, the PKCβ inhibitor is a pharmaceutically acceptable salt of enzastaurin. In one embodiment, the PKCβ inhibitor is a hydrochloride salt of enzastaurin. In one embodiment, the PKCβ inhibitor is a bis-hydrochloride salt of enzastaurin. Enzastaurin has a chemical name of 3-(1-methylindol-3-yl)-4-[1-[1-(pyridin-2-ylmethyl)piperidin-4-yl]indol-3-yl]pyrrole-2,5-dione, and has the structure:

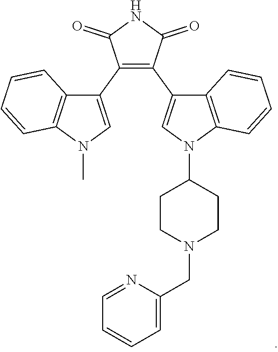 [SMILES: Cn1cc(C2=C(c3cn(C4CCN(Cc5ccccn5)CC4)c4ccccc34)C(=O)NC2=O)c2ccccc21]

In one embodiment, the second active agent used in the methods provided herein is a spleen tyrosine kinase (SYK) inhibitor. In one embodiment, the SYK inhibitor is fostamatinib, or a tautomer, isotopolog, or pharmaceutically acceptable salt thereof. In one embodiment, the SYK inhibitor is fostamatinib. In one embodiment, the SYK inhibitor is a pharmaceutically acceptable salt of fostamatinib. In one embodiment, the SYK inhibitor is fostamatinib disodium hexahydrate. Fostamatinib has a chemical name of (6-((5-fluoro-2-((3,4,5-trimethoxyphenyl)amino)pyrimidin-4-yl)amino)-2,2-dimethyl-3-oxo-2,3-dihydro-4H-pyrido[3,2-b][1,4]oxazin-4-yl)methyl dihydrogen phosphate, and has the structure:

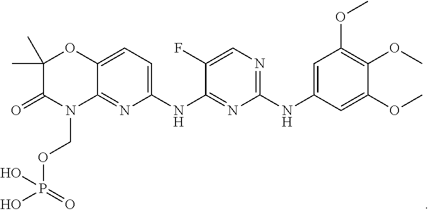

In one embodiment, the second active agent used in the methods provided herein is a Janus kinase 2 (JAK2) inhibitor. In one embodiment, the JAK2 inhibitor is fedratinib, pacritinib, ruxolitinib, baricitinib, gandotinib, lestaurtinib, or momelotinib, or a stereoisomer, mixture of stereoisomers, tautomer, isotopolog, or pharmaceutically acceptable salt thereof.

In one embodiment, the JAK2 inhibitor is fedratinib, or a tautomer, isotopolog, or pharmaceutically acceptable salt thereof. In one embodiment, the JAK2 inhibitor is fedratinib. Fedratinib has a chemical name of N-tert-butyl-3-[(5-methyl-2-{4-[2-(pyrrolidin-1-yl)ethoxy]anilino}pyrimidin-4-yl)amino]benzenesulfonamide, and has the structure:

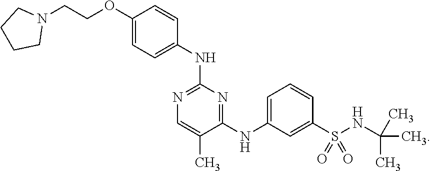 [SMILES: Cc1cnc(Nc2ccc(OCCN3CCCC3)cc2)nc1Nc1cccc(S(=O)(=O)NC(C)(C)C)c1]

In one embodiment, the JAK2 inhibitor is pacritinib, or a tautomer, isotopolog, or pharmaceutically acceptable salt thereof. In one embodiment, the JAK2 inhibitor is pacritinib. Pacritinib has the structure:

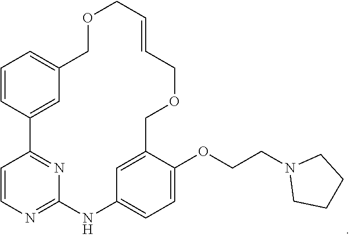 [SMILES: C1=C/COCc2cc(ccc2OCCN2CCCC2)Nc2nccc(n2)-c2cccc(c2)COC/1]

In one embodiment, the JAK2 inhibitor is ruxolitinib, or a stereoisomer, mixture of stereoisomers, tautomer, isotopolog, or pharmaceutically acceptable salt thereof. In one embodiment, the JAK2 inhibitor is ruxolitinib. In one embodiment, the JAK2 inhibitor is a pharmaceutically acceptable salt of ruxolitinib. In one embodiment, the JAK2 inhibitor is ruxolitinib phosphate. Ruxolitinib has a chemical name of (R)-3-(4-(7H-pyrrolo[2,3-d]pyrimidin-4-yl)-1H-pyrazol-1-yl)-3-cyclopentylpropanenitrile, and has the structure:

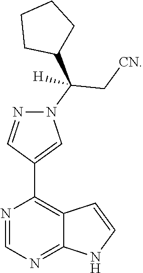 [SMILES: N#CC[C@H](C1CCCC1)n1cc(-c2ncnc3[nH]ccc23)cn1]

In one embodiment, the second active agent used in the methods provided herein is an aurora A kinase inhibitor. In one embodiment, the aurora A kinase inhibitor is alisertib, or a tautomer, isotopolog, or pharmaceutically acceptable salt thereof. In one embodiment, the aurora A kinase inhibitor is alisertib. Alisertib has a chemical name of 4-((9-chloro-7-(2-fluoro-6-methoxyphenyl)-5H-benzo[c]pyrimido[4,5-e]azepin-2-yl)amino)-2-methoxybenzoic acid, and has the structure:

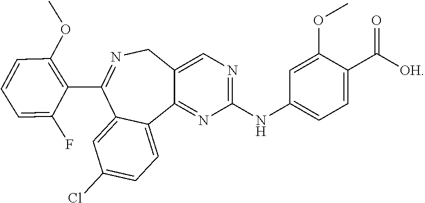 [SMILES: COc1cc(Nc2ncc3c(n2)-c2ccc(Cl)cc2C(c2c(F)cccc2OC)=NC3)ccc1C(=O)O]

In one embodiment, the second active agent used in the methods provided herein is an enhancer of zeste homolog 2 (EZH2) inhibitor. In one embodiment, the EZH2 inhibitor is tazemetostat, GSK126, CPI-1205, 3-deazaneplanocin A (DZNep), EPZ005687, EI1, UNC1999, or sinefungin, or a stereoisomer, mixture of stereoisomers, tautomer, isotopolog, or pharmaceutically acceptable salt thereof.

In one embodiment, the EZH2 inhibitor is tazemetostat, or a tautomer, isotopolog, or pharmaceutically acceptable salt thereof. In one embodiment, the EZH2 inhibitor is tazemetostat. Tazemetostat (also known as EPZ-6438) has a chemical name of N-[(1,2-dihydro-4,6-dimethyl-2-oxo-3-pyridinyl)methyl]-5-[ethyl(tetrahydro-2H-pyran-4-yl)amino]-4-methyl-4′-(4-morpholinylmethyl)-[1,1′-biphenyl]-3-carboxamide, and has the structure:

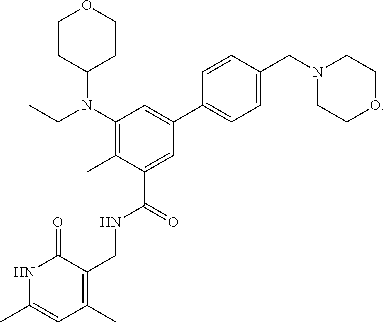 [SMILES: CCN(c1cc(-c2ccc(CN3CCOCC3)cc2)cc(C(=O)NCc2c(C)cc(C)[nH]c2=O)c1C)C1CCOCC1]

In one embodiment, the EZH2 inhibitor is GSK126, or a stereoisomer, mixture of stereoisomers, tautomer, isotopolog, or pharmaceutically acceptable salt thereof. In one embodiment, the EZH2 inhibitor is GSK126 (also known as GSK-2816126). GSK126 has a chemical name of (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide, and has the structure:

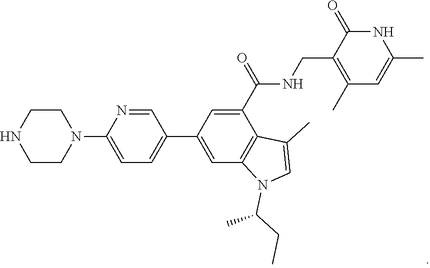 [SMILES: CC[C@H](C)n1cc(C)c2c(C(=O)NCc3c(C)cc(C)[nH]c3=O)cc(-c3ccc(N4CCNCC4)nc3)cc21]

In one embodiment, the EZH2 inhibitor is CPI-1205, or a stereoisomer, mixture of stereoisomers, tautomer, isotopolog, or pharmaceutically acceptable salt thereof. In one embodiment, the EZH2 inhibitor is CPI-1205. CPI-1205 has a chemical name of (R)-N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, and has the structure:

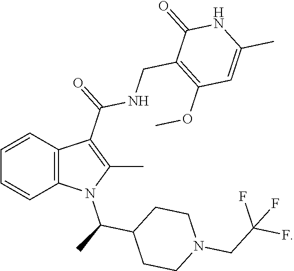 [SMILES: COc1cc(C)[nH]c(=O)c1CNC(=O)c1c(C)n([C@H](C)C2CCN(CC(F)(F)F)CC2)c2ccccc12]

In one embodiment, the EZH2 inhibitor is 3-deazaneplanocin A. In one embodiment, the EZH2 inhibitor is EPZ005687. In one embodiment, the EZH2 inhibitor is EI1. In one embodiment, the EZH2 inhibitor is UNC1999. In one embodiment, the EZH2 inhibitor is sinefungin.

In one embodiment, the second active agent used in the methods provided herein is a bromodomain and extra-terminal motif protein (BET) inhibitor. In one embodiment, the BET inhibitor is birabresib or 4-[2-(cyclopropylmethoxy)-5-(methanesulfonyl)phenyl]-2-methylisoquinolin-1(2H)-one, or a stereoisomer, mixture of stereoisomers, tautomer, isotopolog, or pharmaceutically acceptable salt thereof.

In one embodiment, the BET inhibitor is birabresib, or a stereoisomer, mixture of stereoisomers, tautomer, isotopolog, or pharmaceutically acceptable salt thereof. In one embodiment, the BET inhibitor is birabresib. Birabresib (also known as OTX015 or MK-8628) has a chemical name of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide, and has the structure:

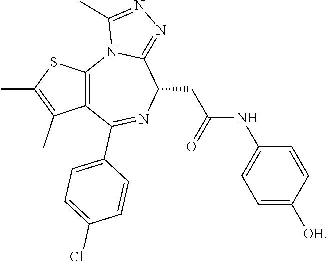 [SMILES: Cc1sc2c(c1C)C(c1ccc(Cl)cc1)=N[C@@H](CC(=O)Nc1ccc(O)cc1)c1nnc(C)n1-2]

In one embodiment, the BET inhibitor is 4-[2-(cyclopropylmethoxy)-5-(methanesulfonyl)phenyl]-2-methylisoquinolin-1(2H)-one, or a tautomer, isotopolog, or pharmaceutically acceptable salt thereof. In one embodiment, the BET inhibitor has the structure:

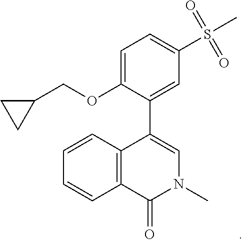 [SMILES: Cn1cc(-c2cc(S(C)(=O)=O)ccc2OCC2CC2)c2ccccc2c1=O]

In one embodiment, the second active agent used in the methods provided herein is a hypomethylating agent. In one embodiment, the hypomethylating agent is 5-azacytidine or decitabine, or a stereoisomer, mixture of stereoisomers, tautomer, isotopolog, or pharmaceutically acceptable salt thereof.

In one embodiment, the hypomethylating agent is 5-azacytidine, or a stereoisomer, mixture of stereoisomers, tautomer, isotopolog, or pharmaceutically acceptable salt thereof. In one embodiment, the hypomethylating agent is 5-azacytidine. 5-Azacytidine has a chemical name of 4-amino-1-β-D-ribofuranosyl-1,3,5-triazin-2(1H)-one, and has the structure:

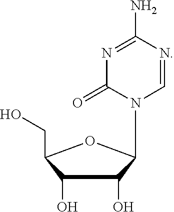

In one embodiment, the hypomethylating agent is decitabine, or a stereoisomer, mixture of stereoisomers, tautomer, isotopolog, or pharmaceutically acceptable salt thereof. In one embodiment, the hypomethylating agent is decitabine. Decitabine has a chemical name of 4-amino-1-(2-deoxy-β-D-erythro-pentofuranosyl)-1,3,5-triazin-2(1H)-one, and has the structure:

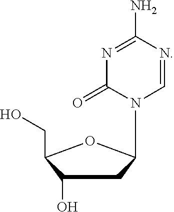 [SMILES: Nc1ncn([C@H]2C[C@@H](O)[C@@H](CO)O2)c(=O)n1]

In certain embodiments, the second active agent used in the methods provided herein is obinutuzumab and the hematological cancer is CLL. In one embodiment, the second active agent includes an administration of a therapeutically effective amount of obinutuzumab in one or more treatment cycles. In one embodiment, obinutuzumab is administered twice every 7 days. In one embodiment, obinutuzumab is administered once every week. In one embodiment, obinutuzumab is administered once every 4 weeks. In one embodiment, obinutuzumab is administered at days 1, 2, 8, and 15 of the first 28-day cycle, and administered at day 1 of the second to the sixth 28-day cycles.

In one embodiment, obinutuzumab is administered at a dose of about 100 mg on day 1 of the first 28-day cycle, about 900 mg on day 2 of the first 28-day cycle, and about 1000 mg on each of days 8 and 15 of the first 28-day cycle. In one embodiment, obinutuzumab is administered at a dose of about 1000 mg combined on day 1 and 2 of the first 28-day cycle, and about 1000 mg on each of days 8 and 15 of the first 28-day cycle. In one embodiment, obinutuzumab is administered at a dose of about 1000 mg on day 1 of the second to the sixth 28-day cycles.

In one embodiment, the second active agent used in the methods provided herein is obinutuzumab and comprises administering to a patient a therapeutically effective amount of a compound of Formula (I), in combination with a second active agent provided herein (e.g., venetoclax), and further in combination with obinutuzumab.

In one embodiment, the second active agent used in the methods provided herein is a DOT1L inhibitor. In one embodiment, the DOT1L inhibitor is pinometostat, or a stereoisomer, mixture of stereoisomers, tautomer, isotopolog, or pharmaceutically acceptable salt thereof. In one embodiment, the DOT1L inhibitor is pinometostat. Pinometostat (also known as EPZ-5676) has a chemical name of (2R,3R,4S,5R)-2-(6-amino-9H-purin-9-yl)-5-((((1r,3S)-3-(2-(5-(tert-butyl)-1H-benzo[d]imidazol-2-yl)ethyl)cyclobutyl)(isopropyl)amino)methyl)tetrahydrofuran-3,4-diol, and has the structure:

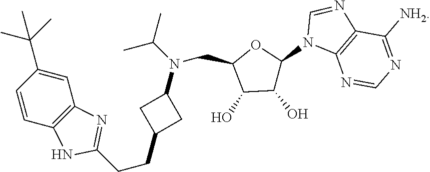

In one embodiment, the second active agent used in the methods provided herein is a histone acetyltransferase (HAT) inhibitor. In one embodiment, the HAT inhibitor is C646, or a tautomer, isotopolog, or pharmaceutically acceptable salt thereof. In one embodiment, the HAT inhibitor is C646. C646 has a chemical name of 4-(4-((5-(4,5-dimethyl-2-nitrophenyl)furan-2-yl)methylene)-3-methyl-5-oxo-4,5-dihydro-1H-pyrazol-1-yl)benzoic acid, and has the structure:

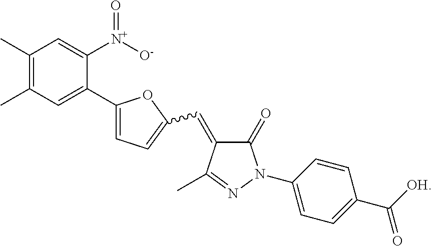 [SMILES: CC1=NN(c2ccc(C(=O)O)cc2)C(=O)C1=Cc1ccc(-c2cc(C)c(C)cc2[N+](=O)[O-])o1]

In one embodiment, the second active agent used in the methods provided herein is a WD repeat-containing protein 5 (WDR5) inhibitor. In one embodiment, the WDR5 inhibitor is OICR-9429, or a tautomer, isotopolog, or pharmaceutically acceptable salt thereof. In one embodiment, the WDR5 inhibitor is OICR-9429. OICR-9429 has a chemical name of N-(4-(4-methylpiperazin-1-yl)-3′-(morpholinomethyl)-[1,1′-biphenyl]-3-yl)-6-oxo-4-(trifluoromethyl)-1,6-dihydropyridine-3-carboxamide, and has the structure:

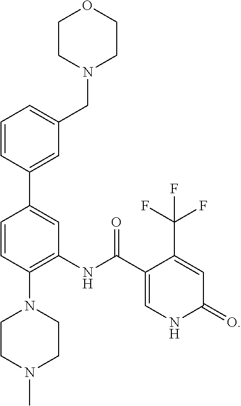 [SMILES: CN1CCN(c2ccc(-c3cccc(CN4CCOCC4)c3)cc2NC(=O)c2c[nH]c(=O)cc2C(F)(F)F)CC1]

In one embodiment, the second active agent used in the methods provided herein is a DNA (cytosine-5)-methyltransferase 1 (DNMT1) inhibitor. In one embodiment, the DNMT1 inhibitor is a DNMT1 selective inhibitor. In one embodiment, the DNMT1 selective inhibitor is GSK3484862, or a stereoisomer, mixture of stereoisomers, tautomer, isotopolog, or pharmaceutically acceptable salt thereof. In one embodiment, the DNMT1 selective inhibitor is GSK3484862. GSK3484862 (also known as GSKMI-714) has a chemical name of (R)-2-((3,5-dicyano-6-(dimethylamino)-4-ethylpyridin-2-yl)thio)-2-phenylacetamide, and has the structure:

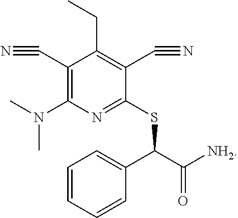 [SMILES: CCc1c(C#N)c(S[C@@H](C(N)=O)c2ccccc2)nc(N(C)C)c1C#N]

In one embodiment, the second active agent used in the methods provided herein is a lysine-specific demethylase 1 (LSD-1) inhibitor. In one embodiment, the LDS-1 inhibitor is Compound C or seclidemstat, or a tautomer, isotopolog, or pharmaceutically acceptable salt thereof.

In one embodiment, the LSD-1 inhibitor is Compound C, or a tautomer, isotopolog, or pharmaceutically acceptable salt thereof. In one embodiment, the LSD-1 inhibitor is Compound C. In one embodiment, the LSD-1 inhibitor is a pharmaceutically acceptable salt of Compound C. In one embodiment, the LSD-1 inhibitor is Compound C besylate. In one embodiment, the LSD-1 inhibitor is Compound C mono-besylate. Compound C has a chemical name of 4-(2-(4-aminopiperidin-1-yl)-5-(3-fluoro-4-methoxyphenyl)-1-methyl-6-oxo-1,6-dihydropyrimidin-4-yl)-2-fluorobenzonitrile, and has the structure:

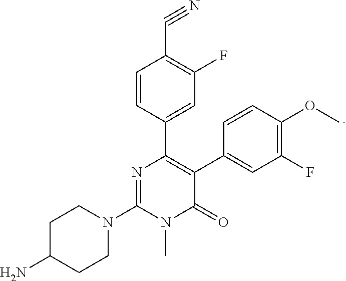 [SMILES: COc1ccc(-c2c(-c3ccc(C#N)c(F)c3)nc(N3CCC(N)CC3)n(C)c2=O)cc1F]

In one embodiment, the LSD-1 inhibitor is seclidemstat, or a tautomer, isotopolog, or pharmaceutically acceptable salt thereof. In one embodiment, the LSD-1 inhibitor is seclidemstat. In one embodiment, the LSD-1 inhibitor is a pharmaceutically acceptable salt of seclidemstat. In one embodiment, the LSD-1 inhibitor is seclidemstat mesylate. Seclidemstat (also known as SP-2577) has a chemical name of (E)-N′-(1-(5-chloro-2-hydroxyphenyl)ethylidene)-3-((4-methylpiperazin-1-yl)sulfonyl)benzohydrazide, and has the structure:

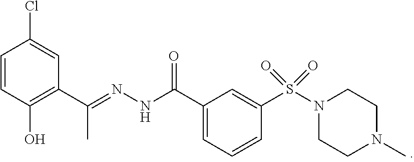 [SMILES: C/C(=N\NC(=O)c1cccc(S(=O)(=O)N2CCN(C)CC2)c1)c1cc(Cl)ccc1O]

In one embodiment, the second active agent used in the methods provided herein is a G9A (one of the histone H3 methyltransferases) inhibitor. In one embodiment, the G9A inhibitor is UNC0631, or a tautomer, isotopolog, or pharmaceutically acceptable salt thereof. In one embodiment, the G9A inhibitor is UNC0631. UNC0631 has a chemical name of N-(1-(cyclohexylmethyl)piperidin-4-yl)-2-(4-isopropyl-1,4-diazepan-1-yl)-6-methoxy-7-(3-(piperidin-1-yl)propoxy)quinazolin-4-amine, and has the structure:

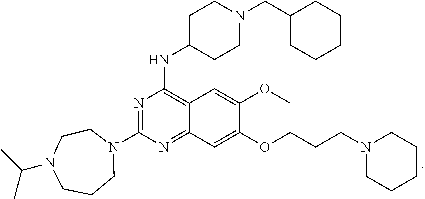 [SMILES: COc1cc2c(NC3CCN(CC4CCCCC4)CC3)nc(N3CCCN(C(C)C)CC3)nc2cc1OCCCN1CCCCC1]

In one embodiment, the second active agent used in the methods provided herein is a protein arginine methyltransferase 5 (PRMT5) inhibitor. In one embodiment, the PRMT5 inhibitor is GSK3326595, or a stereoisomer, mixture of stereoisomers, tautomer, isotopolog, or pharmaceutically acceptable salt thereof. In one embodiment, the PRMT5 inhibitor is GSK3326595. GSK3326595 (also known as EPZ-015938) has a chemical name of (S)-6-((1-acetylpiperidin-4-yl)amino)-N-(3-(3,4-dihydroisoquinolin-2(1H)-yl)-2-hydroxypropyl)pyrimidine-4-carboxamide, and has the structure:

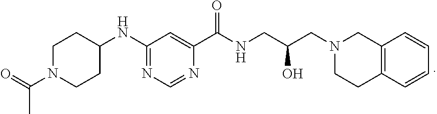 [SMILES: CC(=O)N1CCC(Nc2cc(C(=O)NC[C@H](O)CN3CCc4ccccc4C3)ncn2)CC1]

In one embodiment, the second active agent used in the methods provided herein is a bromodomain (BRD) inhibitor. In one embodiment, the BRD inhibitor is a BRD9/7 inhibitor. In one embodiment, the BRD9/7 inhibitor is LP99, or a stereoisomer, mixture of stereoisomers, tautomer, isotopolog, or pharmaceutically acceptable salt thereof. In one embodiment, the BRD9/7 inhibitor is LP99. LP99 has a chemical name of N-((2R,3S)-2-(4-chlorophenyl)-1-(1,4-dimethyl-2-oxo-1,2-dihydroquinolin-7-yl)-6-oxopiperidin-3-yl)-2-methylpropane-1-sulfonamide, and has the structure:

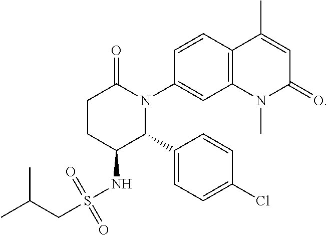 [SMILES: Cc1cc(=O)n(C)c2cc(N3C(=O)CC[C@H](NS(=O)(=O)CC(C)C)[C@H]3c3ccc(Cl)cc3)ccc12]

In one embodiment, the second active agent used in the methods provided herein is a SUV420H1/H2 (two homologous enzymes that methylate lysine 20 of histone H4) inhibitor. In one embodiment, the SUV420H1/H2 inhibitor is A-196, or a tautomer, isotopolog, or pharmaceutically acceptable salt thereof. In one embodiment, the SUV420H1/H2 inhibitor is A-196. A-196 has a chemical name of 6,7-dichloro-N-cyclopentyl-4-(pyridin-4-yl)phthalazin-1-amine, and has the structure:

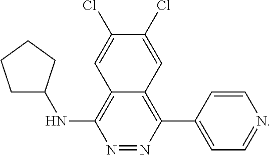 [SMILES: Clc1cc2c(NC3CCCC3)nnc(-c3ccncc3)c2cc1Cl]

In one embodiment, the second active agent used in the methods provided herein is a coactivator-associated arginine methyltransferase 1 (CARM1) inhibitor. In one embodiment, the CARM1 inhibitor is EZM2302, or a stereoisomer, mixture of stereoisomers, tautomer, isotopolog, or pharmaceutically acceptable salt thereof. In one embodiment, the CARM1 inhibitor is EZM2302. EZM2302 has a chemical name of methyl (R)-2-(2-(2-chloro-5-(2-hydroxy-3-(methylamino)propoxy)phenyl)-6-(3,5-dimethylisoxazol-4-yl)-5-methylpyrimidin-4-yl)-2,7-diazaspiro[3.5]nonane-7-carboxylate, and has the structure:

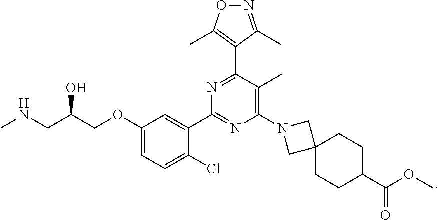

In one embodiment, the second active agent used in the methods provided herein is a chemotherapy. In one embodiment, the chemotherapy is bendamustine, doxorubicin, etoposide, methotrexate, cytarabine, vincristine, ifosfamide, melphalan, oxaliplatin, dexamethasone or a stereoisomer, mixture of stereoisomers, tautomer, isotopolog, prodrug, or pharmaceutically acceptable salt thereof.

In one embodiment, the chemotherapy is bendamustine, or a tautomer, isotopolog, or pharmaceutically acceptable salt thereof. In one embodiment, the chemotherapy is bendamustine. In one embodiment, the chemotherapy is a pharmaceutically acceptable salt of bendamustine. In one embodiment, the chemotherapy is bendamustine hydrochloride. In one embodiment, the chemotherapy is a mono-hydrochloride salt of bendamustine. Bendamustine has a chemical name of 4-(5-(bis(2-chloroethyl)amino)-1-methyl-1H-benzo[d]imidazol-2-yl)butanoic acid, and has the structure:

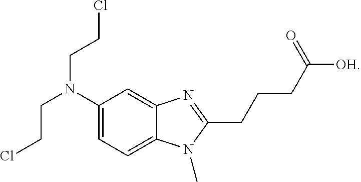 [SMILES: Cn1c(CCCC(=O)O)nc2cc(N(CCCl)CCCl)ccc21]

In one embodiment, the chemotherapy is doxorubicin, or a stereoisomer, mixture of stereoisomers, tautomer, isotopolog, or pharmaceutically acceptable salt thereof. In one embodiment, the chemotherapy is doxorubicin. In one embodiment, the chemotherapy is a pharmaceutically acceptable salt of doxorubicin. In one embodiment, the chemotherapy is doxorubicin hydrochloride. In one embodiment, the chemotherapy is a mono-hydrochloride salt of doxorubicin. Doxorubicin has the structure:

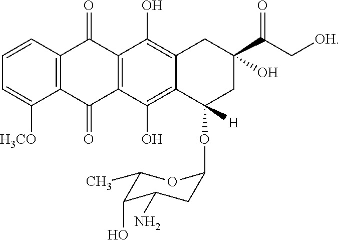 [SMILES: COc1cccc2c1C(=O)c1c(O)c3c(c(O)c1C2=O)C[C@@](O)(C(=O)CO)C[C@@H]3OC1CC(N)C(O)C(C)O1]

In one embodiment, the chemotherapy is etoposide, or a stereoisomer, mixture of stereoisomers, tautomer, isotopolog, prodrug, or pharmaceutically acceptable salt thereof. In one embodiment, the chemotherapy is etoposide. Etoposide has a chemical name of 4′-demethylepipodophyllotoxin 9-[4,6-O-(R)-ethylidene-β-D-glucopyranoside], and has the structure:

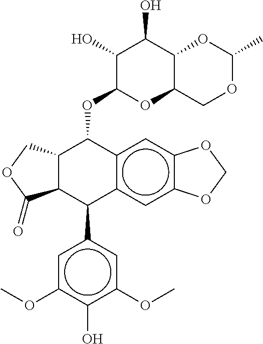

In one embodiment, the chemotherapy is a prodrug of etoposide. In one embodiment, the chemotherapy is an ester prodrug of etoposide. In one embodiment, the chemotherapy is etoposide phosphate. Etoposide phosphate has a chemical name of 4′-demethylepipodophyllotoxin 9-[4,6-O-(R)-ethylidene-β-D-glucopyranoside], 4′ (dihydrogen phosphate), and has the structure:

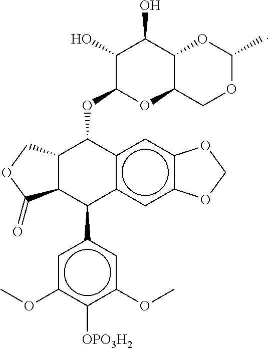

In one embodiment, the chemotherapy is methotrexate, or a stereoisomer, mixture of stereoisomers, tautomer, isotopolog, or pharmaceutically acceptable salt thereof. In one embodiment, the chemotherapy is methotrexate. In one embodiment, the chemotherapy is a pharmaceutically acceptable salt of methotrexate. In one embodiment, the chemotherapy is methotrexate sodium. Methotrexate has a chemical name of (4-(((2,4-diaminopteridin-6-yl)methyl)(methyl)amino)benzoyl)-L-glutamic acid, and has the structure:

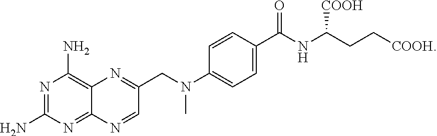 [SMILES: CN(Cc1cnc2nc(N)nc(N)c2n1)c1ccc(C(=O)N[C@@H](CCC(=O)O)C(=O)O)cc1]

In one embodiment, the chemotherapy is cytarabine, or a stereoisomer, mixture of stereoisomers, tautomer, isotopolog, or pharmaceutically acceptable salt thereof. In one embodiment, the chemotherapy is cytarabine. Cytarabine has a chemical name of 4-amino-1-β-D-arabinofuranosyl-2(1H)pyrimidinone, and has the structure:

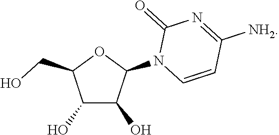 [SMILES: Nc1ccn([C@@H]2O[C@H](CO)[C@@H](O)[C@@H]2O)c(=O)n1]

In one embodiment, the chemotherapy is vincristine, or a stereoisomer, mixture of stereoisomers, tautomer, isotopolog, or pharmaceutically acceptable salt thereof. In one embodiment, the chemotherapy is vincristine. In one embodiment, the chemotherapy is a pharmaceutically acceptable salt of vincristine. In one embodiment, the chemotherapy is vincristine sulfate. In one embodiment, the chemotherapy is a mono-sulfate salt of vincristine. Vincristine has the structure:

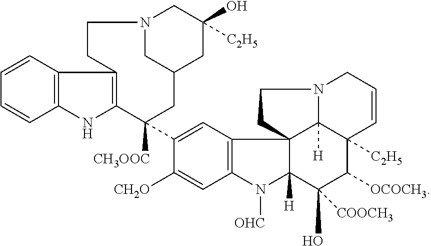

In one embodiment, the chemotherapy is ifosfamide, or a tautomer, isotopolog, or pharmaceutically acceptable salt thereof. In one embodiment, the chemotherapy is ifosfamide. Ifosfamide has a chemical name of 3-(2-chloroethyl)-2-[(2-chloroethyl)amino]tetrahydro-2H-1,3,2-oxazaphosphorine 2-oxide, and has the structure:

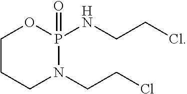 [SMILES: O=P1(NCCCl)OCCCN1CCCl]

In one embodiment, the chemotherapy is melphalan, or a stereoisomer, mixture of stereoisomers, tautomer, isotopolog, or pharmaceutically acceptable salt thereof. In one embodiment, the chemotherapy is melphalan. In one embodiment, the chemotherapy is a pharmaceutically acceptable salt of melphalan. In one embodiment, the chemotherapy is melphalan hydrochloride. In one embodiment, the chemotherapy is a mono-hydrochloride salt of melphalan. Melphalan has a chemical name of 4-[bis(2-chloroethyl)amino]-L-phenylalanine, and has the structure:

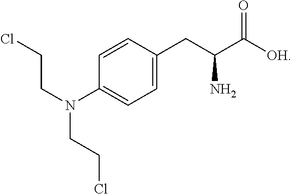 [SMILES: N[C@@H](Cc1ccc(N(CCCl)CCCl)cc1)C(=O)O]

In one embodiment, the chemotherapy is oxaliplatin, or a stereoisomer, mixture of stereoisomers, tautomer, isotopolog, or pharmaceutically acceptable salt thereof. In one embodiment, the chemotherapy is oxaliplatin. Oxaliplatin has a chemical name of cis-[(1R,2R)-1,2cyclohexanediamine-N,N′][oxalato(2-)-O,O′]platinum, and has the structure:

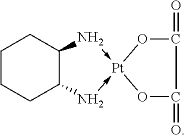

In one embodiment, the chemotherapy is dexamethasone, or a stereoisomer, mixture of stereoisomers, tautomer, isotopolog, or pharmaceutically acceptable salt thereof. In one embodiment, the chemotherapy is dexamethasone. Dexamethasone has a chemical name of (11b,16a)-9-fluoro-11,17,21-trihydroxy-16-methylpregna-1,4-diene-3,20-dione, and has the structure:

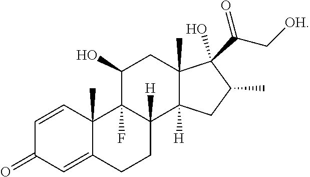

In certain embodiments, the second therapeutic agent is administered before, after or simultaneously with a compound of Formula (I). Administration of a compound of Formula (I) and a second therapeutic agent to a patient can occur simultaneously or sequentially by the same or different routes of administration. The suitability of a particular route of administration employed for a particular second drug or agent will depend on the second therapeutic agent itself (e.g., whether it can be administered orally or topically without decomposition prior to entering the blood stream) and the subject being treated. Particular routes of administration for the second drug or agents or ingredients are known to those of ordinary skill in the art. See, e.g., The Merck Manual, 448 (17th ed., 1999).

Any combination of the above therapeutic agents, suitable for treatment of the diseases or symptoms thereof, can be administered. Such therapeutic agents can be administered in any combination with a compound of Formula (I) or an enantiomer, mixture of enantiomers, tautomer, isotopolog, or pharmaceutically acceptable salt thereof, at the same time or as a separate course of treatment.

As used herein, the term “in combination” does not restrict the order in which therapies (e.g., prophylactic and/or therapeutic agents) are administered to a patient with a disease or disorder. In one embodiment, a first therapy (e.g., a prophylactic or therapeutic agent such as a compound provided herein, e.g., Compound 1, Compound 2, or Compound 3, or an enantiomer, mixture of enantiomers, tautomer, isotopolog, or pharmaceutically acceptable salt thereof) is administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before) the administration of a second therapy (e.g., a second active agent provided herein). In one embodiment, a first therapy (e.g., a prophylactic or therapeutic agent such as a compound provided herein, e.g., Compound 1, Compound 2, or Compound 3, or an enantiomer, mixture of enantiomers, tautomer, isotopolog, or pharmaceutically acceptable salt thereof) is administered concomitantly with the administration of a second therapy (e.g., a second active agent provided herein). In one embodiment, a first therapy (e.g., a prophylactic or therapeutic agent such as a compound provided herein, e.g., Compound 1, Compound 2, or Compound 3, or an enantiomer, mixture of enantiomers, tautomer, isotopolog, or pharmaceutically acceptable salt thereof) is administered subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a second active agent provided herein).

Administration of Compound 1, Compound 2, or Compound 3, or an enantiomer, mixture of enantiomers, tautomer, isotopolog, or pharmaceutically acceptable salt thereof, and a second active agent provided herein, to a patient can occur simultaneously or sequentially by the same or different routes of administration. The suitability of a particular route of administration employed for a particular active agent will depend on the active agent itself (e.g., whether it can be administered orally without decomposing prior to entering the blood stream).

Certain embodiments of the invention are illustrated by the following non-limiting examples. It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### 6. EXAMPLES

The examples below are carried out using standard techniques, which are well known and routine to those of skill in the art, except where otherwise described in detail. The examples are intended to be merely illustrative.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

### Example 1: Synthesis of 2-(2,6-Dioxopiperidin-3-yl)-4-((2-fluoro-4-((3-morpholinoazetidin-1-yl)methyl)benzyl)amino)isoindoline-1,3-dione (Compound 3)

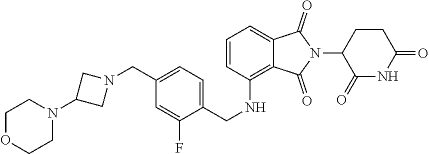 [SMILES: O=C1CCC(N2C(=O)c3cccc(NCc4ccc(CN5CC(N6CCOCC6)C5)cc4F)c3C2=O)C(=O)N1]

To a solution of 4-((4-(chloromethyl)-2-fluorobenzyl)amino)-2-(2,6-dioxopiperidin-3-yl)isoindoline-1,3-dione (215 mg, 0.500 mmol) (prepared as described herein) and 4-(azetidin-3-yl)morpholine hydrochloride (107 mg, 0.600 mmol) in dry DMSO (1.7 mL) was added DIEA (262 μL, 1.50 mmol) and the mixture stirred at ambient temperature for 48 hours. The reaction mixture was diluted with 20% formic acid in DMSO (2.5 mL) and filtered through a membrane syringe filter (0.45 μm nylon). The solution was purified using standard methods to provide 2-(2,6-dioxopiperidin-3-yl)-4-((2-fluoro-4-((3-morpholinoazetidin-1-yl)methyl)benzyl)amino)isoindoline-1,3-dione (173 mg, 64.6% yield). LCMS (ESI) m/z 536.2 [M+H]+.

### Example 2: Synthesis of (S)-2-(2,6-Dioxopiperidin-3-yl)-4-((2-fluoro-4-((3-morpholinoazetidin-1-yl)methyl)benzyl)amino)isoindoline-1,3-dione (Compound 1)

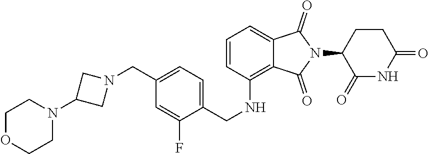 [SMILES: O=C1CC[C@H](N2C(=O)c3cccc(NCc4ccc(CN5CC(N6CCOCC6)C5)cc4F)c3C2=O)C(=O)N1]

### (S)-2-(2,6-Dioxopiperidin-3-yl)-4-((2-fluoro-4-(hydroxymethyl)benzyl)amino)isoindoline-1,3-dione

A suspension of (S)-4-amino-2-(2,6-dioxopiperidin-3-yl)isoindoline-1,3-dione (5.00 g, 18.3 mmol) and 2-fluoro-4-(hydroxymethyl)benzaldehyde (2.82 g, 18.30 mmol) in 2:1 dioxane-MeOH (75 mL) was cooled to 0° C. and B₁₀H₁₄ (4.92 g, 40.3 mmol) was added in small portions over 5 minutes. The reaction flask was fitted with a septum and needle vent (pressure) and vigorously stirred for 10 minutes. The mixture was allowed to reach ambient temperature and stirred for 3 hours. The mixture was concentrated and the residue purified by silica gel chromatography (0-10% MeOH-DCM) to provide (S)-2-(2,6-dioxopiperidin-3-yl)-4-((2-fluoro-4-(hydroxymethyl)benzyl)amino)isoindoline-1,3-dione as a yellow solid (4.23 g, 56%). LCMS (ESI) m/z 411.8 [M+H]⁺.

### (S)-4-((4-(Chloromethyl)-2-fluorobenzyl)amino)-2-(2,6-dioxopiperidin-3-yl)isoindoline-1,3-dione

A solution of (S)-2-(2,6-dioxopiperidin-3-yl)-4-((2-fluoro-4-(hydroxymethyl)benzyl)amino)isoindoline-1,3-dione (0.727 g, 1.77 mmol) in dry NMP (6 mL) was cooled to 0° C. and methane sulfonyl chloride (0.275 mL, 3.35 mmol) and DIEA (0.617 mL, 3.53 mmol) were added sequentially. The reaction mixture was allowed to reach ambient temperature and was stirred for 18 hours. The reaction mixture was slowly added to H₂O (60 mL) cooled to 0° C. with vigorous mixing. The resulting suspension was filtered and the collected solid was washed with H₂O and Et₂O. The solid was dissolved in EtOAc and the solution dried with MgSO₄, filtered and concentrated to provide (S)-4-((4-(chloromethyl)-2-fluorobenzyl)amino)-2-(2,6-dioxopiperidin-3-yl)isoindoline-1,3-dione as a yellow solid (0.600 g, 79%). LCMS (ESI) m/z 430.0 [M+H]⁺.

### (S)-2-(2,6-Dioxopiperidin-3-yl)-4-((2-fluoro-4-((3-morpholinoazetidin-1-yl)methyl)benzyl)amino)isoindoline-1,3-dione

To a solution of (S)-4-((4-(chloromethyl)-2-fluorobenzyl)amino)-2-(2,6-dioxopiperidin-3-yl)isoindoline-1,3-dione (300 mg, 0.698 mmol) in dry DMSO (1.0 mL) was added 4-(azetidin-3-yl)morpholine hydrochloride (125 mg, 0.698 mmol) and DIEA (0.122 mL, 0.698 mmol). The reaction mixture was stirred at ambient temperature for 18 hours and was diluted with DMSO (1 mL). The solution was purified by chiral reverse-phase chromatography to give (S)-2-(2,6-dioxopiperidin-3-yl)-4-((2-fluoro-4-((3-morpholinoazetidin-1-yl)methyl)benzyl)amino)isoindoline-1,3-dione (89 mg, 24%, 97% ee). LCMS (ESI) m/z 536.2 [M+H]⁺.

### Example 3: Synthesis of (R)-2-(2,6-Dioxopiperidin-3-yl)-4-((2-fluoro-4-((3-morpholinoazetidin-1-yl)methyl)benzyl)amino)isoindoline-1,3-dione (Compound 2)

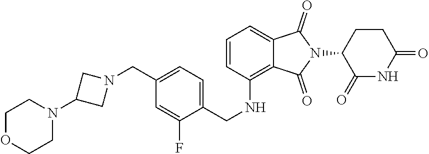 [SMILES: O=C1CC[C@@H](N2C(=O)c3cccc(NCc4ccc(CN5CC(N6CCOCC6)C5)cc4F)c3C2=O)C(=O)N1]

The chiral reverse-phase chromatography described in Example 2 additionally provided (R)-2-(2,6-dioxopiperidin-3-yl)-4-((2-fluoro-4-((3-morpholinoazetidin-1-yl)methyl)benzyl)amino)isoindoline-1,3-dione (16 mg, 97% ee) LCMS (ESI) m/z 535.6 [M+H]⁺.

### Example 4: Compound 1 Induced Apoptosis and Inhibited Proliferation in Diffuse Large B-Cell Lymphoma Cell Lines

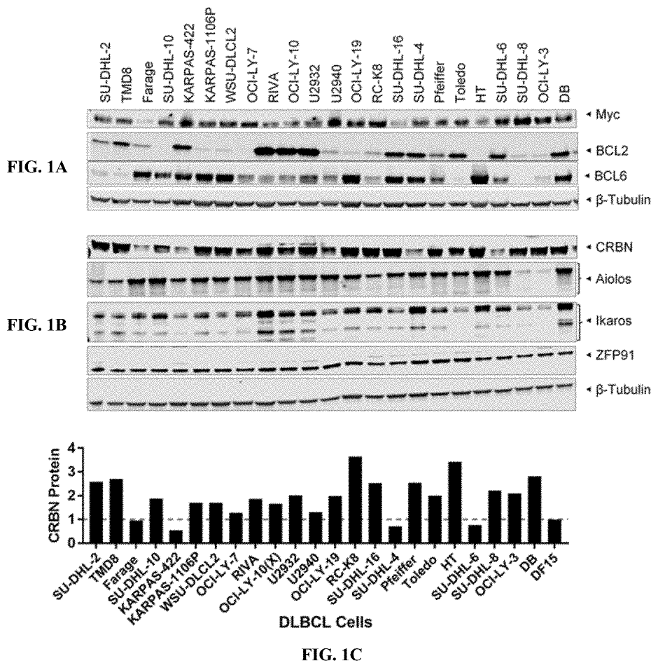

Compound 1 activity was evaluated in a panel of twenty three DLBCL cell lines. The DLBCL panel includes seven cell lines classified as activated B-cell (ABC) subtype, thirteen cell lines classified as the germinal center B-cell (GCB) subtype, and three cell lines classified as primary mediastinal B-cell lymphoma (PMBL). In addition, many of these cell lines displayed similar cytogenetic (concurrent rearrangement of MYC and/or BCL2 or/and BCL6) and molecular characteristics observed in high-risk DLBCL patients. Thus, eleven cell lines have a t(8;14)(q24;q32) and/or t(14;18)(q32;q21.3), corresponding to MYC and BCL2 gene rearrangements into an immunoglobulin (IG) heavy chain locus as well as additional rearrangements affecting BCL6 (Table 1). The translocation of MYC, BCL6, or BCL2 to an IG locus usually leads to high levels of mRNA and protein due to the active transcription driven by a constitutively active IG promoter. Therefore, the protein levels of these genes were monitored by Western blots, confirming the overexpression of BCL2, MYC, and/or BCL6 in all the cell lines evaluated (FIG. 1, panel A). Expressions of CRBN protein and known substrates, Ikaros, Aiolos, and ZFP91 were also evaluated (FIG. 1, panels B and C).

TABLE 1
Diffuse Large B-cell Lymphoma Cell Line Characteristics

| | DLBCL | | | | |
|---|---|---|---|---|---|
| Cell Line | Subtype | MYC | BCL2 | BCL6 | Relevant Partial Karyotype |
| TMD8(1) | ABC | ND | ND | ND | No consistent abnormality |
| | | | | | detected |
| SU-DHL-10 | GCB | +IG | +IG | ND | t(8;14)(q24;q32), |
| | | | | | t(14;18)(q32;q21); |
| | | | | | carries concurrent |
| | | | | | rearrangements of IGH with |
| | | | | | MYC and BCL2 |
| SU-DHL-2 | ABC | ND | ND | ND | No consistent abnormality |
| | | | | | detected |
| Karpa-422 | GCB | ND | +IG | ND | t(14;18) IGH-BCL2 |
| | | | | | t(4;11)(q21;q23) MLL/AF4 |
| SU-DHL-8 | GCB | +IG | ND | ND | t(8;22)(q24;q11); carries |
| | | | | | t(8;22) effecting IGL-MYC |
| | | | | | rearrangement) |
| Farage | PMBL | ND | ND | ND | No consistent abnormality |
| | | | | | detected |
| OCI-Ly-7 | GCB | +IG | +AMP | +AMP | t(8;14)(q24;q32), |
| | | | | | del(18)(q21.1); carries t(8;14) |
| | | | | | affecting MYC-IGH |
| | | | | | rearrangement |
| SU-DHL-6 | GCB | +non- | +IG | ND | t(8;9)(q24;p13), |
| | | IG | | | t(8;19;9)(q24;q13;p13), |
| | | | | | t(14;18)(q32;q21) |
| Karpas-1106P | PMBL | ND | ND | ND | No consistent abnormality |
| | | | | | detected |
| WSU-DLCL-2 | GCB | +non- | +IG | +non- | t(3;8)(q27;q24), |
| | | IG | | IG | t(14;18)(q32;q21) |
| OCI-Ly10 | ABC | +AMP | +AMP | ND | No additional |
| | | | | | chromosomal |
| | | | | | rearrangements |
| | | | | | detected |
| U-2940 | PMBL | ND | ND | ND | Carries homozygous |
| | | | | | microdeletion of SOCS1 |
| | | | | | locus |
| U-2932 | ABC | ND | +AMP | +AMP | Extensive genomic |
| | | | | | amplification of BCL2 and |
| | | | | | BCL6 region |
| RIVA | ABC | +non- | +AMP | ND | t(4;8)(q22;q24); |
| | | IG | | | amp(18)(q21)dup(18)(q21q23) |
| SU-DHL-16 | GCB | ND | +IG | ND | t(14;18)(q32;q21) translocation |
| RC-K8 | ABC | ND | ND | +non- | t(3;7)(q27;q32) ; |
| | | | | IG | t(11;14)(q23;q32); |
| | | | | | carries breakpoints at REL |
| | | | | | and IGH-DDX6 |
| HT | GCB | ND | ND | ND | no IGH-BCL2 or other |
| | | | | | relevant rearrangements |
| | | | | | detected |
| SU-DHL-4 | GCB | +non- | +IG | +non- | t(1;3)(q42;q27), |
| | | IG | | IG | t(14;18)(q32;q21) |
| Toledo | GCB | ND | +IG | ND | t(14;18)(q32;q21) |
| OCI-LY-19 | GCB | +non- | +IG | +AMP | t(4;8)(q32;q24), |
| | | IG | | | t(14;18)(q32;q21) |
| Pfeiffer | GCB | ND | + IG | ND | t(14;18)(q32;q21) |
| OCI-LY-3 | ABC | +AMP | +AMP | +AMP | t(4;18) with copy number |
| | | | | | amplification of the BCL2 |
| | | | | | region |
| DB | GCB | +non- | +IG | +non- | t(3;8)(q27;q24), |
| | | IG | | IG | t(14;18)(q32;q21) |

ABC = activated B-cell;
DLBCL = diffuse large B-cell lymphoma;
GCB = germinal center B-cell;
PMBL = primary mediastinal B-cell lymphoma;
+AMP = gene amplification;
ND = no detected chromosomal rearrangements.

TABLE 2
Antiproliferative Activity and Apoptotic Effect of Compound 1 in Diffuse Large B-cell Lymphoma Cell Lines

| | % Viable Cells | | | | Apoptosis | | | |
|---|---|---|---|---|---|---|---|---|
| Cell Line | AUC | IC₅₀ | Eₘₐₓ | Sensitivity | AUC | EC₅₀ | Eₘₐₓ | Sensitivity |
| SU-DHL-10 | 3.371 | 0.01 | 2.477 | High | 815.3 | 0.01634 | 81.92 | High |
| TMD8 | 3.911 | 0.003 | 1.365 | High | 826.8 | 0.004443 | 83.15 | High |
| SU-DHL-2 | 6.871 | 0.0013 | 1.264 | High | 745 | 0.007779 | 75.15 | High |
| KARPAS-422 | 45.21 | 0.0054 | 1.584 | High | 682.2 | 0.0325 | 70.47 | High |
| SU-DHL-8 | 54.43 | 0.25 | 2.508 | High | 652.5 | 0.509 | 70.52 | High |
| Farage | 59.29 | 0.0037 | 1.676 | High | 503.6 | 0.07361 | 54.17 | High |
| OCI-LY-7 | 61.98 | 0.0036 | 1.657 | High | 673.2 | 0.01278 | 68.82 | High |
| SU-DHL-6 | 88.38 | 0.26 | 5.843 | High | 421.1 | 0.452 | 46.26 | High |
| KARPAS-1106P | 110.9 | 0.025 | 1.183 | High | 597.7 | 0.05612 | 59.77 | High |
| WSU-DLCL2 | 173.8 | 0.02 | 1.36 | High | 464.5 | 0.07701 | 47.81 | High |
| OCI-Ly10 | 267.3 | 0.05 | 19.19 | Intermediate | 187.7 | 2.093 | 29.41 | Intermediate |
| U2940 | 289.1 | 0.035 | 29.24 | Intermediate | 267.4 | 0.0679 | 29.04 | Intermediate |
| U2932 | 327.1 | 0.1 | 24.81 | Intermediate | 280.5 | 11.94 | 69.92 | Intermediate |
| RIVA | 395.8 | 0.05 | 36.99 | Intermediate | 216.6 | 0.01897 | 22.67 | Intermediate |
| SU-DHL-16 | 359.7 | 0.12 | 30.33 | Intermediate | 159.1 | 0.2615 | 18.2 | Intermediate |
| RC-K8 | 409.1 | 0.1 | 34.92 | Intermediate | 81.59 | 0.2301 | 9.528 | Intermediate |
| HT | 434.8 | 0.47 | 45.18 | Intermediate | 158.2 | 0.007821 | 16.58 | Intermediate |
| SU-DHL-4 | 457.4 | 0.5 | 41.79 | Intermediate | 155.2 | 0.4522 | 18.92 | Intermediate |
| Toledo | 507 | 0.5 | 52.68 | Intermediate | 114.8 | 0.5 | 13.97 | Intermediate |
| OCI-LY-19 | 283.3 | 0.03 | 28.11 | Intermediate | 293.5 | 0.08092 | 30.64 | Intermediate |
| Pfeiffer | 912.3 | NA | 86.19 | Limited | 133.5 | 0.3 | 8.01 | Limited |
| OCI-LY-3 | 986.1 | NA | 84.33 | Limited | 7.009 | 0.00046 | 0.7533 | Limited |
| DB | 1173 | NA | 107.1 | Limited | 0 | NA | NA | Limited |

AUC = area under the curve; IC₅₀ = 50% inhibitory concentration; Eₘₐₓ = maximum efficacy achieved; NA = Not Achieved.

The panel of cell lines were treated with increasing doses of compounds for five days, at which time the number of viable cells was measured and the amount of apoptosis was determined by 7-AAD exclusion and Annexin V staining using flow cytometry. Table 2 shows that eighteen of the twenty three lines evaluated were highly sensitive to Compound 1 in vitro after five days of treatment, with IC₅₀ values between 0.001 and 0.4 μM. Overall, the sensitivity to Compound 1 was independent of cell of origin (COO), since ABC, GCB, and PMBL subtypes were sensitive to Compound 1 (Table 2). Additionally, Compound 1 showed potent antiproliferative activity in lines with MYC, BCL2, and BCL6 chromosomal translocations and/or with high protein expression levels for these genes, suggesting the potential for broad activity of Compound 1 across a range of DLBCL subtypes (FIG. 1; Table 2).

The dose-response curves, indicating the loss of viable cells induced by Compound 1 across the panel of DLBCL cell lines, delineated three categories of response (Table 2). Ten cell lines were highly sensitive to Compound 1 with less than 6% viable cells remaining (Eₘₐₓ) after treatment; ten cell lines showed intermediate sensitivity with 15% to 55% cell viability, and three cell lines had limited or no response to Compound 1 (Table 2). To further characterize the antiproliferative effect of Compound 1, Annexin V and 7-AAD staining were measured as indicators of apoptosis. Dramatic induction of apoptosis was observed in the same cell lines identified as sensitive to Compound 1 in the cell growth assays (Table 2).

The results further demonstrated that the antiproliferative effects of Compound 1 did not correlate with the absolute level of baseline cereblon expression. In some cell lines, the inhibition of cell growth was of similar magnitude even though the levels of cereblon protein expression were different (FIG. 1, panels B and C).

Taken together, these results demonstrate that Annexin V and 7-AAD staining can be measured as indicators of apoptosis, and that these markers correlate with activity of Compound 1.

### Example 5: Compound 1 Inhibited Proliferation and Induced Apoptosis in Drug-Resistant Diffuse Large B-Cell Lymphoma Cell Lines

Compound 1 activity was tested in Diffuse Large B-cell Lymphoma (DLBCL) cell lines that were resistant to therapeutic agents used in the clinic to treat the disease. To this effect, the patterns of sensitivity or resistance of Compound 1 was compared to doxorubicin, venetoclax, and ibrutinib (three drugs of known activity in DLBCL). The activity of Compound 1 was also evaluated in cell lines with acquired resistance to doxorubicin.

To begin, the activity of Compound 1 was compared to that of drugs currently used in DLBCL treatment by exposing the same panel of twenty three cell lines (Table 1) to doxorubicin, venetoclax, and ibrutinib. Most cell lines exhibited distinct patterns of responsiveness to Compound 1, doxorubicin, ibrutinib, and venetoclax (Table 3). All DLBCL cell lines were strongly sensitive to doxorubicin, including the three cell lines that are insensitive to Compound 1 (Table 3). Nine of the Compound 1-sensitive cell lines were resistant to venetoclax and ten of the Compound 1-sensitive cell lines were resistant to ibrutinib, while four strongly Compound 1-sensitive cell lines (SU-DHL-2, Farage, RIVA, WSU-DLBCL2) were resistant to both venetoclax and ibrutinib (Table 3). This demonstrates that Compound 1 was effective in cell lines that are resistant or refractory to drugs currently used in DLBCL treatment, such as Ventoclax and Ibrutinib.

TABLE 3
IC₅₀ Concentrations for Compound 1, Doxorubicin, Ventoclax
and Ibrutinib in Diffuse Large B-cell Lymphoma Cell Lines

| | IC₅₀ (μM) | | | |
|---|---|---|---|---|
| Cell Line | Compound 1 | Doxorubicin | Venetoclax | Ibrutinib |
| SU-DHL-10 | 3.4 | 0.6 | 454 | 128 |
| TMD8 | 4 | 0.7 | 390 | 9 |
| SU-DHL-2 | 7 | 1.3 | 355 | 496 |
| KARPAS-422 | 45 | 1.0 | 75 | 182 |
| SU-DHL-8 | 54 | 0.6 | 548 | 360 |
| Farage | 59 | 1.2 | 560 | 104 |
| OCI-LY-7 | 62 | 5 | 1008 | 763 |
| SU-DHL-6 | 88 | 3 | 174 | 216 |
| KARPAS-1106P | 111 | 1.7 | 200 | 211 |
| WSU-DLCL2 | 174 | 1.5 | 215 | 161 |
| OCI-Ly10 | 267 | 3 | 200 | 532 |
| OCI-LY-19 | 283 | 2 | 300 | 400 |
| U2940 | 289 | 2 | 706 | 367 |
| U2932 | 327 | 3 | 230 | 362 |
| SU-DHL-16 | 360 | 1.5 | 21 | 92 |
| RIVA | 396 | 5.5 | 5 | 173 |
| RC-K8 | 409 | 3.5 | 609 | 1061 |
| HT | 435 | 2 | 644 | 483 |
| SU-DHL-4 | 457 | 1 | 145 | 303 |
| Toledo | 507 | 4 | 500 | 847 |
| Pfeiffer | 855 | 3 | 432 | 725 |
| OCI-LY-3 | 986 | 11.6 | 1023 | 915 |
| DB | 1173 | 3.8 | 82 | 1016 |

The activity of Compound 1 was also evaluated in cell lines with acquired resistance to doxorubicin (Table 4). Doxorubicin-resistant cell lines (DoxoR) were generated by culturing the parental cell lines in vitro with increasing concentrations of doxorubicin for a long period of time (˜9 to 18 months) until they were able to grow in the presence of a relatively high concentration of doxorubicin (1 μM). Matching parental (M-Parent) cells were generated by maintaining the parental cells in culture without treatment for the same amount of time (˜9 to 18 months). The growth-inhibitory effect of doxorubicin on parental cells and the corresponding resistant cells was evaluated by measuring the number of viable cells and extent of apoptosis by 7-AAD exclusion and Annexin V staining using flow cytometry. The IC₅₀ values and cell growth inhibition curves for doxorubicin are shown in Table 4. In the DoxoR cell lines, the growth-inhibitory effects of doxorubicin were greatly decreased in comparison with its effects on the matching parental cells. The shifts in IC₅₀ values for doxorubicin in DoxoR lines were more than 100-fold (Table 4).

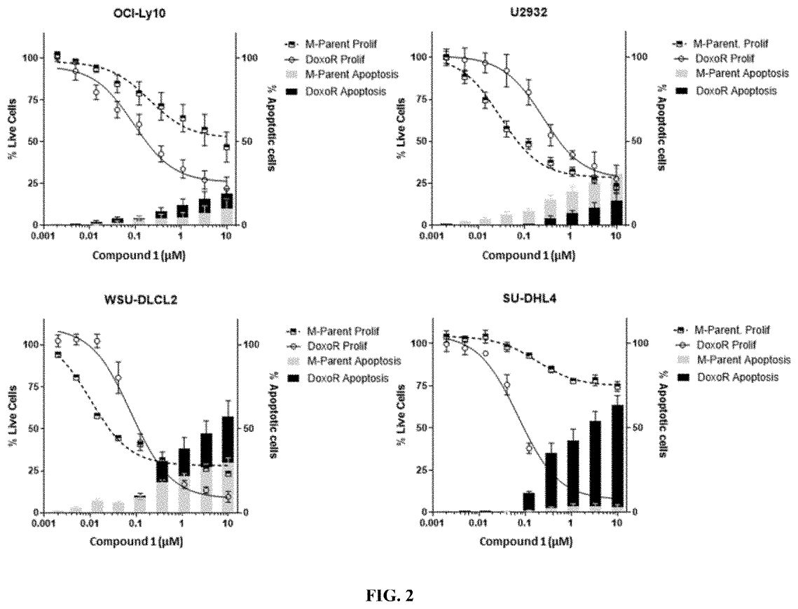

The effects of Compound 1 on the viability of doxorubicin-resistant versus parental cell lines was determined (FIG. 2; Table 5). An approximately 20-fold greater sensitivity to Compound 1 was measured for resistant OCI-Ly10 cells compared to parental cells (IC₅₀ of 6 μM in parental cells compared with an IC₅₀ of 0.3 μM in resistant cells), and a >100-fold greater sensitivity to Compound 1 was observed in the doxorubicin-resistant SU-DHL-4 cell line (from IC₅₀ of >10 μM to 0.1 μM).

The increased antiproliferative activity observed for Compound 1 in OCI-Ly10 and SU-DHL-4 resistant cells was accompanied by an increase in the capacity of Compound 1 to induce apoptosis. In WSU-DLCL2 cells, Compound 1 induced more apoptosis (as reflected in higher Emax) in the DoxoR cell line than in the M-parental line, particularly at higher concentrations (0.1 to 1 μM), despite the lower potency in the DoxoR line (IC₅₀=0.1 μM) relative to the M-parental cell line (IC₅₀=0.01 μM). In U2932 doxorubicin-resistant cells, a 20-fold lower sensitivity to Compound 1, as well as lower apoptosis induction, was observed in comparison with the M-parental cell line (FIG. 2; Table 5).

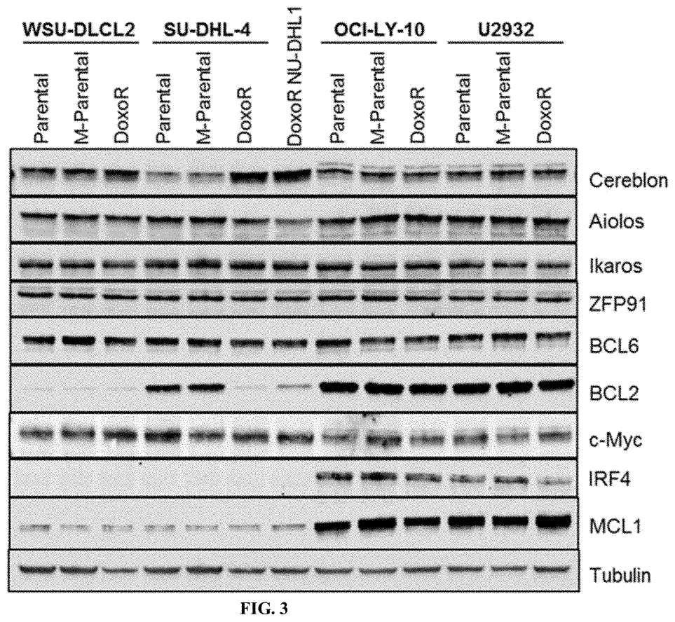

Western blot experiments (FIG. 3) indicated that the increased apoptotic effect of Compound 1 in SU-DHL-4 doxorubicin-resistant cells correlated with the loss of BCL2 expression in this cell line. These results suggested that BCL2 expression can serve as a marker of response to Compound 1, including in drug resistant cell lines.

In summary, Compound 1 was efficacious in certain drug resistant cell lines. Compound 1 demonstrated dose-dependent antiproliferative responses and cell killing responses in all doxorubicin-resistant cell lines tested indicating that cross-resistance to Compound 1 might not be common after acquiring resistance to doxorubicin. A lack of cross-resistance between Compound 1 and venetoclax or ibrutinib was also demonstrated. Furthermore, markers of apoptosis, such as Annexin V and 7-AAD staining and protein expression of Bcl-2, can indicate responsiveness to Compound 1.

TABLE 4
Antiproliferative Activity of Doxorubicin in Doxorubicin-resistant and
Parental Diffuse Large B-cell Lymphoma Cell Lines

| | | Doxorubicin |
|---|---|---|
| Cell Line | | IC₅₀ (μM) |
| Oci-Ly10 | M-Parental | 0.02 |
| | DoxoR | 1.4 |
| U2932 | M-Parental | 0.01 |
| | DoxoR | 1.08 |
| WSU-DLCL2 | M-Parental | 0.01 |
| | DoxoR | 1.6 |
| SU-DHL-4 | M-Parental | 0.01 |
| | DoxoR | 1.5 |

DoxoR = doxorubicin-resistant;
IC₅₀ = 50% inhibitory concentration;
M-Parental = matching parental.

TABLE 5
Compound 1 Antiproliferative Activity in Diffuse Large B-cell
Lymphoma Cell Lines Sensitive and Resistant to Doxorubicin

| Cell Line | | IC₅₀ (μM) | Eₘₐₓ (% viable cells remaining) |
|---|---|---|---|
| Oci-Ly 10 | M-Parental | 6 | 47 |
| | DoxoR | 0.3 | 22 |
| U2932 | M-Parental | 0.06 | 19 |
| | DoxoR | 1.5 | 31 |
| WSU-DLCL2 | M-Parental | 0.01 | 23 |
| | DoxoR | 0.1 | 9 |
| SU-DHL-4 | M-Parental | >10 | 74 |
| | DoxoR | 0.1 | 8 |

DoxoR = doxorubicin-resistant;
Eₘₐₓ = maximal inhibitory response relative to DMSO;
IC₅₀ = 50% inhibitory concentration;
M-Parental = matching parental.

### Example 6: Compound 1 Exhibited Selective Anti-Inflammatory, Immunomodulatory, and Fibrosis and Matrix Remodeling Activities in Primary Monoculture and Co-culture Systems

The activity of Compound 1 was profiled in a panel of human primary cell-based assays, modeling complex tissue and disease biology of organs (vasculature, immune system, skin, lung) and general tissue biology using the BioMAP System (DiscoveRx, Fremont, Calif.).

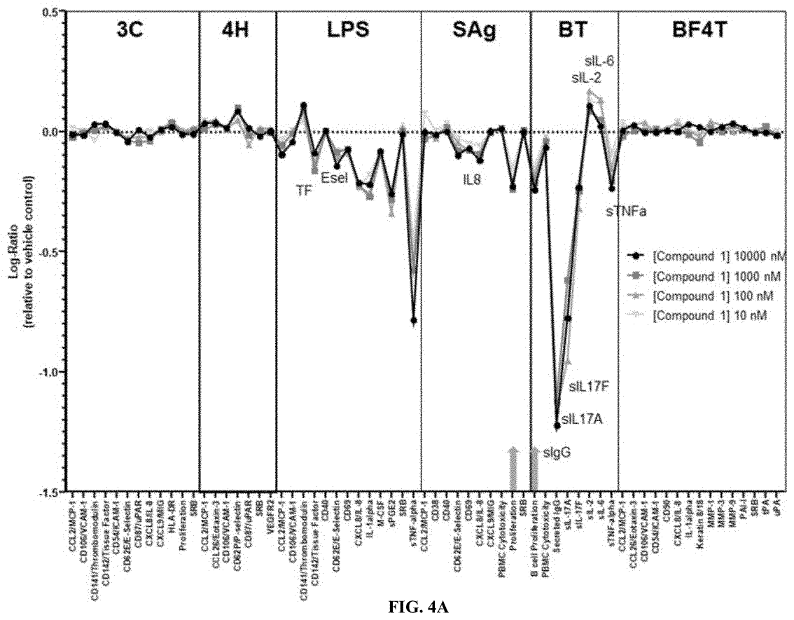

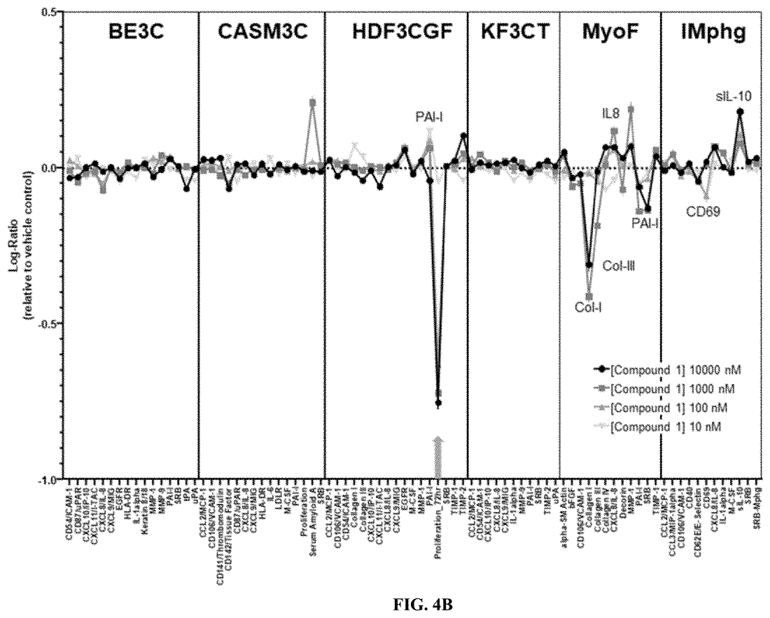

The BioMAP System consists of twelve primary human monoculture or co-culture systems in stimulated and non-stimulated control conditions (FIG. 4, panel A). When tested at 0.01, 0.1, 1, and 10 μM, Compound 1 mediated changes in key biomarker activities. Specifically, the profiles for Compound 1 reflected selective anti-inflammatory and immunomodulatory impact on monocyte (LPS) and T cell dependent B cell activation responses (BT). Treatment with Compound 1 led to a decrease in interleukin 8 (IL-8), interleukin 1a (IL-1a), secreted prostaglandin E2 (sPGE2), and secreted tumor necrosis factor alpha (sTNFα) in the LPS system, which consisted of peripheral blood mononuclear cells (PBMCs) and endothelial cells. Furthermore, secreted IgG (sIgG), secreted interleukin 17A (sIL-17A), secreted interleukin 17F (sIL-17F), and sTNFα decreased, whereas secreted interleukin 2 (sIL-2) and secreted interleukin 6 (sIL-6) increased, in the BT system, which was comprised of B cells and PBMCs. In addition, Compound 1 exhibited a compelling inhibition of collagen-I and -III expression, as well as slight inhibition of plasminogen activator inhibitor-1 (PAI-1) expression, in the MyoF system, comprised of lung fibroblasts, modeling fibrosis and matrix remodeling-related biology.

In conclusion, Compound 1 profiles in the BioMAP Diversity PLUS panel indicated that the compound exhibited anti-inflammatory, immunomodulatory, and fibrosis and matrix remodeling activities in primary monoculure and co-culture systems. Furthermore, this demonstrated that expression of IL-8, IL-1a, sPGE2, sTNFα, sIgG, sIL-17A, sIL-17F, sIL-2, sIL-6, collagen-I and —III, and PAI-1 can serve as biomarkers for response to Compound 1 treatment.

### Example 7: Antiproliferative Activity of Compound 1 is Dependent on Cereblon

Cereblon works as a substrate receptor for a CRL4 ubiquitin E3 ligase and the binding of cereblon modulating (CM) compounds induces the recruitment, ubiquitination, and destruction of key target substrates such as Ikaros, Aiolos and ZFP91 to mediate cellular effects.

To determine whether the strong antiproliferative activity of Compound 1 demonstrated in DLBCL cell lines depends on binding to cereblon, CRISPR/Cas9 gene editing technology was used to ablate the expression of cereblon. The activated B-cell (ABC) and MYC/BCL2 double expressor cell lines SU-DHL-2 and RIVA, the germinal center B-cell (GCB) and MYC/BCL2 double hit cell lines (and expressing BCL6) Karpas-422, SU-DHL-10, WSU-DLCL2, and the primary mediastinal B-cell lymphoma (PMBL) cell line Farage were infected with a CAS9 expressing lentivirus construct to generate the control CAS9 expressing cells. The Cas9 cells had wild type cereblon expression (CRBN^{WT}).

To generate the cereblon knock out cells (CRBN^{-/-}), the individual control CAS9 cells were infected with a construct expressing a subgenomic single guide RNA (sgRNA). Western blot analysis confirmed the absence of cereblon protein in the CRBN^{-/-} cells, relative to the levels of the housekeeping protein β-tubulin. The CRBN^{WT} and CRBN^{-/-} cells were treated with increasing concentrations of Compound 1 for five days. The results demonstrated that control CAS9 expressing cells (CRBN^{WT}) were sensitive to Compound 1 (IC₅₀ ranging between 2.5 and 40 nM; Table 6), whereas CRBN knock-out blocked the antiproliferative activity of Compound 1 (Table 6), indicating that the antiproliferative effect of Compound 1 on DLBCL cells is cereblon-dependent. These results indicate that CRBN can serve as a biomarker for treatment with Compound 1.

TABLE 6
Effect of Compound 1 on Cell Proliferation in CRBN^{WT} and
CRBN^{-/-} Diffuse Large B-cell Lymphoma Cell Lines

| | CRBN^{WT} cells | | CRBN^{-/-} cells |
|---|---|---|---|
| | IC₅₀ (nM) | Eₘₐₓ (%) | IC₅₀ (nM) |
| SU-DHL2 | 2.5 | 1 | 10,000< |
| RIVA | 8 | 30 | 10,000< |
| Karpas-422 | 15 | 5 | 10,000< |
| SU-DHL10 | 35 | 0 | 10,000< |
| WSU-DLCL2 | 40 | 19 | 10,000< |
| Farage | 8 | 0.5 | 10,000< |

IC₅₀ = 50% inhibitory concentration;
Eₘₐₓ = maximum response achievable.

### Example 8: Compound 1 Treatment of Diffuse Large B-Cell Lymphoma Caused Rapid Loss of Aiolos, Ikaros, and ZFP91 and Induced Apoptosis In Vitro

The effect of Compound 1 on the cellular degradation of known cereblon substrate proteins was tested in engineered cell lines. Using an enzyme fragment complementation (EFC) assay, DF15 cell lines expressing ePL-tagged target protein substrates of Aiolos, Ikaros, and ZFP91 were monitored for degradation at 1, 2, 6, and 24 hours over a concentration range from 6 pM to 10 μM. Measurement of Ikaros, Aiolos, and ZFP91 protein levels were evaluated (Table 7). After treatment with various concentrations of Compound 1 for different periods of time, the remaining ePL-tagged substrates in the cells were measured using a luminescence plate reader. In these assays, Compound 1 induced a time- and concentration-dependent loss of Aiolos (Table 7), Ikaros (Table 7), and ZFP91 (Table 7). Compound 1 showed high potency (low nanomolar or below nanomolar EC50 values (Table 7) for degradation of Aiolos, Ikaros, and ZFP91 at all time points 1, 2, 6, and 24 hours following addition of compound to the cultures. However, Compound 1 achieved maximum efficacy (Emax) degrading the three substrates (1% to 10% remaining substrate) after 6 hours treatment and at concentrations as low as 15 nM. The kinetics of the degradation did not change over the 24-hour time course and maximal effect on degradation was sustained up to the last time point of 24 hours (Table 7).

The results of cell-based degradation assays using tagged recombinant proteins of known cereblon substrates indicated that Compound 1 is a potent and efficient “degrader” of Ikaros, Aiolos, and ZFP91 (Table 7), and that these substrates serve as useful biomarkers for evaluating the activity of Compound 1.

TABLE 7
Analysis of the Concentration Response Curves for Compound
1-Induced Degradation of Substrates at Different Timepoints.

| | Aiolos | | Ikaros | | ZFP91 | |
|---|---|---|---|---|---|---|
| Timepoint | EC₅₀ | Eₘₐₓ | EC₅₀ | Eₘₐₓ | EC₅₀ | Eₘₐₓ |
| (hours) | (nM) | (%) | (nM) | (%) | (nM) | (%) |
| 1 | 0.6 | 60 | 0.6 | 75.5 | 1 | 64 |
| 2 | 0.5 | 16.5 | 0.8 | 33 | 0.5 | 26.5 |
| 6 | 0.2 | 6 | 0.5 | 1.5 | 0.2 | 4 |
| 24 | 0.2 | 7 | 0.4 | 0.03 | 0.1 | 2.5 |

EC₅₀ = concentration of a drug that gives half-maximal response; Eₘₐₓ = maximum response achievable; ZFP91 = zinc finger protein 91.

### Example 9: Compound 1 Promoted the Degradation of Endogenous Substrates in DLBCL Cell Lines in a Cereblon-Dependent Manner and Induced Apoptosis

To evaluate whether Compound 1 can promote the degradation of endogenous substrates in DLBCL cell lines in a cereblon-dependent manner and to demonstrate the potential mechanism of induction of apoptosis, a time- and concentration-response study was performed with Compound 1 in the activated B-cell (ABC) lines, SU-DHL-2^{CRBNWT} CAS9 control cells, and the cereblon knockout SU-DHL2^{CRBN-/-} cells.

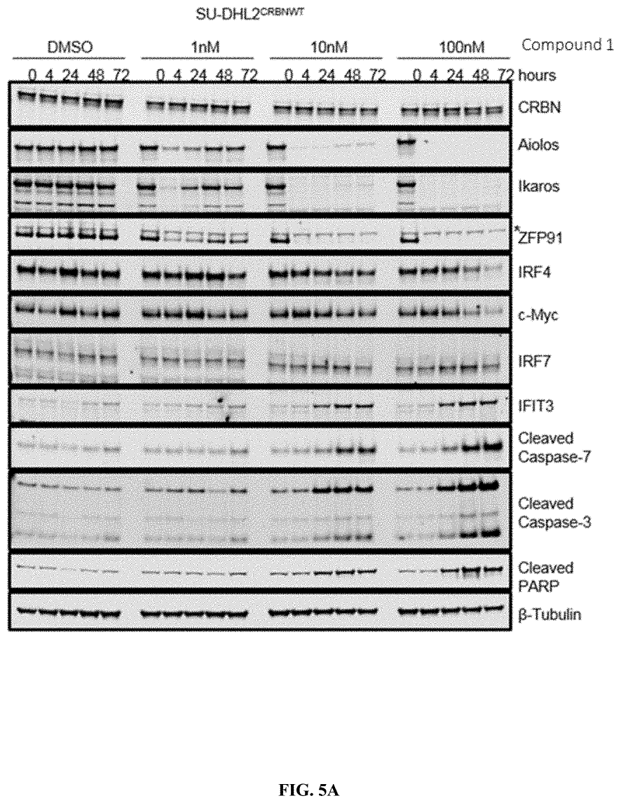

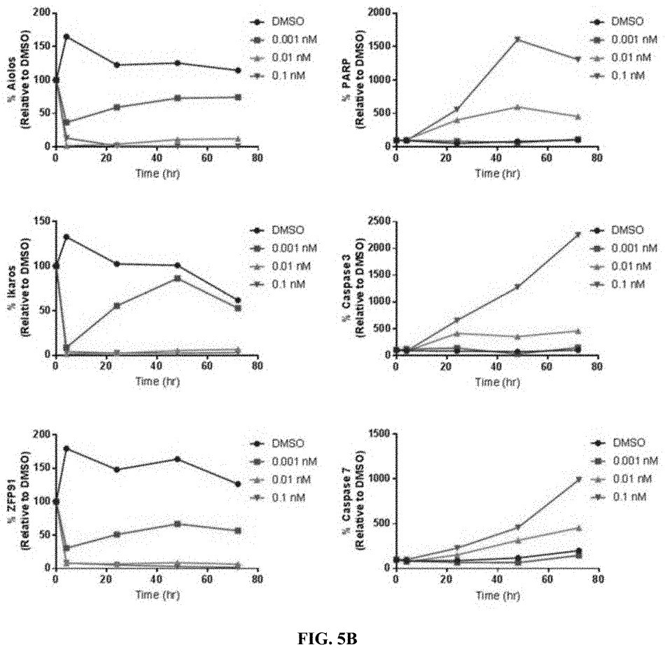

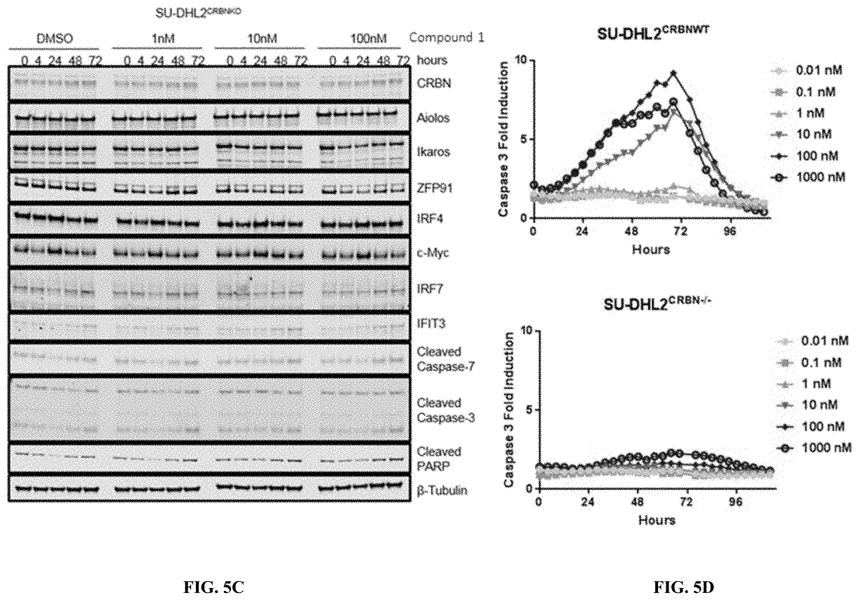

Treatment with Compound 1, at doses as low as 1 nM, resulted in loss of endogenous Ikaros, Aiolos, and ZFP91 proteins in a time- and concentration-dependent manner in SU-DHL-2^{CRBNWT} cells, but not in SU-DHL-2^{CRBN-/-} cells, indicating the requirement of cereblon for the Compound 1-induced degradation of endogenous substrates in DLBCL (FIG. 5 panel A, panel B, and panel C). Time course analysis revealed that exposure to Compound 1 led to rapid degradation of Aiolos, Ikaros, and ZFP91 as early as 4 hours after treatment with all concentrations and that exposures to 10 and 100 nM concentrations of Compound 1 resulted in complete suppression of Ikaros, Aiolos, and ZFP91 protein expression throughout the entire course of treatment. At these two concentrations and concomitant with the strong suppression of the cereblon substrates, Compound 1 treatment induced the interferon-stimulated genes IRF7 and IFIT3, reduced the expression of MYC and IRF4, and induced expression of apoptotic markers [cleaved caspases 3 and 7 and cleaved poly (ADP-ribose) polymerase (PARP)] as early as 24 hours after treatment (FIG. 5, panels A and B). However, Compound 1 had no activity in SU-DHL-2^{CRBN-/-} cells, indicating the requirement of cereblon for induction of apoptosis by Compound 1 in DLBCL (FIG. 5 C).

To confirm the timing of induction of apoptosis and its cereblon dependency, a live-cell imager that provides kinetic, real-time caspase-3 activation (indicator of apoptosis) data was used. SU-DHL-2^{CRBNWT} cells treated with Compound 1 were analyzed for caspase-3 activation by following the cleavage in cells of a specific caspase-3 enzyme substrate over a time course of 112 hours. Compound 1-dependent induction of caspase-3 activity began at 12 hours post treatment and reached maximal induction at ˜72 hours (FIG. 5 D). These results confirm cereblon dependency and the rapid induction of apoptosis in DLBCL cells by Compound 1 at concentrations of 10 nM or above.

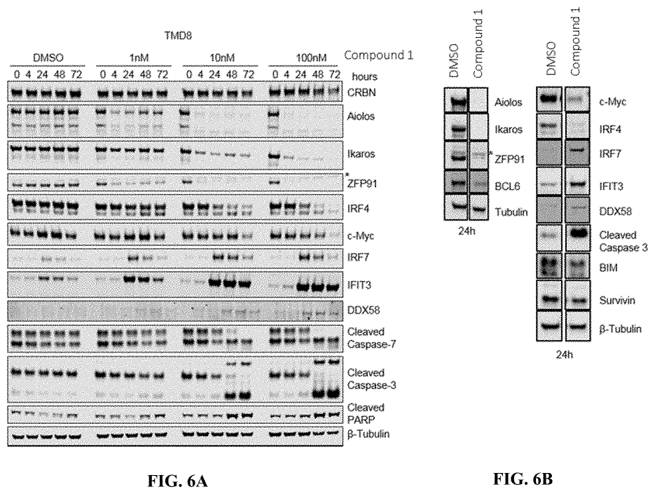

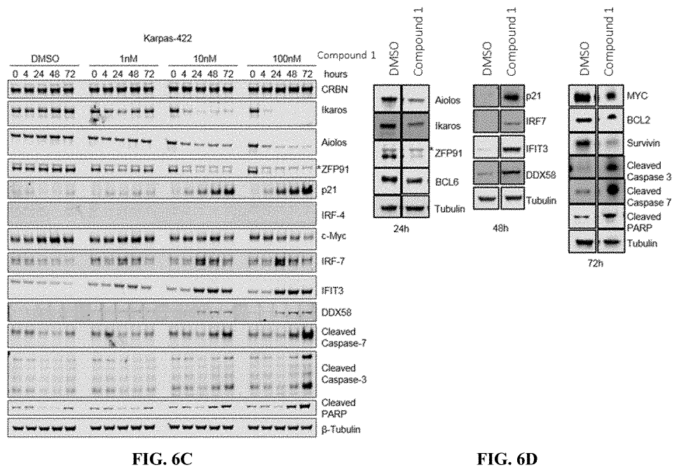

To confirm that the rapid degradation of substrates and induction of apoptosis by Compound 1 is not unique to SU-DHL2 cells, TMD8 (ABC cell line) and Karpas-422 (GCB-cell line) cells were treated with vehicle control (DMSO) or Compound 1 at the times and concentrations indicated (FIG. 6). Time course analysis reveals that exposure to Compound 1 led to rapid degradation of Ikaros and ZFP91 as early as 4 hours after treatment with all concentrations of Compound 1. Exposures to 10 and 100 nM Compound 1 resulted in complete suppression of Ikaros, Aiolos, and ZFP91 protein expression throughout the entire course of treatment (FIG. 6, panel A and C). At these two concentrations, and concomitant with the strong suppression of the cereblon substrates, Compound 1 treatment induced the interferon-stimulated genes DDX58, IRF7, and IFIT3, reduced the expression of MYC and IRF4 (only in TMD8 since the GCB line Karpas-442 does not express IRF4), and induced expression of apoptotic markers as early as 24 hours (TMD8) and at 48 hours (Karpas-422) (FIG. 6, panel A and C).

In a second experiment, we confirmed that Compound 1 (100 nM) treatment of TMD8 produced a large reduction of Aiolos, Ikaros, and ZFP91 at the 24-hour timepoint. Additionally, Compound 1 dramatically decreased the abundance of BCL6 (65% reduction), MYC (75% reduction), and IRF4 (90% reduction), and produced a strong induction of cleaved-caspase 3 (FIG. 6, panel B), confirming the rapid induction of cell death as early as 24 hours in TMD8 cells (FIG. 6, panel B). In Karpas-422 cells, Compound 1 also produced a reduction of Aiolos, Ikaros, and ZFP91 at the 24-hour timepoint (FIG. 6, panel D). At the 48-hour timepoint, Compound 1 increased the abundance of the proliferation inhibitor p21 and the interferon-stimulated genes IRF7, IFIT3, and DDX58, and reduced the expression of MYC (FIG. 6, panel D). After 72 hours, Compound 1-treated cells showed decreased MYC (50% reduction), anti-apoptotic BCL2, and survivin protein levels, as well as strong induction of the apoptosis markers cleaved caspases 3 and 7, and cleaved poly (ADP-ribose) polymerase (PARP) (FIG. 6, panel D).

All together, these data indicate that binding of Compound 1 to cereblon results in nearly complete degradation of Ikaros, Aiolos, and ZFP91, leading to strong and rapid induction of apoptosis in DLBCL cells. Moreover, these data demonstrate that in addition to cereblon, Ikaros, Aiolos, and ZFP91, the interferon-stimulated genes DDX58, IRF7, and IFIT3, the apoptotic related proteins BCL2, survivin, cleaved caspases 3 and 7, and cleaved poly (ADP-ribose) polymerase (PARP), as well as the expression of BCL6, MYC and IRF4, can serve as markers of Compound 1 response.

### Example 10: Multiple Cereblon Substrates Mediated the Cytotoxic Effects of Compound 1

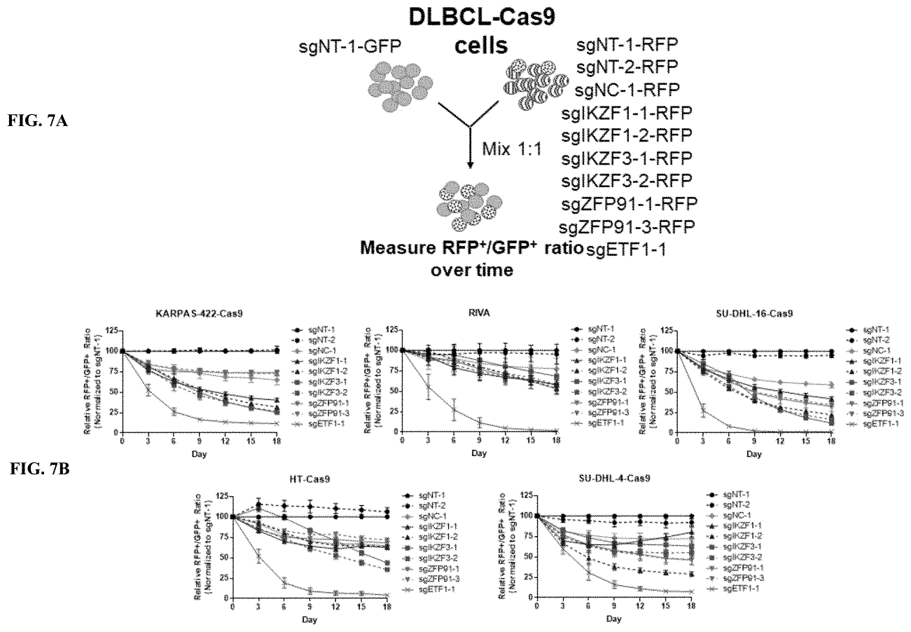

Compound 1 induces autonomous cell killing activity in DLBCL cells in a cereblon-dependent manner. To investigate the consequence of loss of a single cereblon substrate, CRISPR/Cas9-mediated knockout of a cereblon substrate coupled with a flow cytometry-based cellular competition assay was utilized to assess relative cell fitness upon gene knock-out in six DLBCL cell lines: KARPAS-422, U-2932, RIVA, SU-DHL-16, HT, and SU-DHL-4 (FIG. 7, Panel A). To perform the flow cytometry-based competition assay, stable Cas9-expressing DLBCL cells were transduced with a control non-targeting sgRNA construct containing a GFP reporter (sgNT-1-GFP), or with a sgRNA construct targeting a gene of interest containing an RFP reporter (sgRNA-RFP). For targeted knock-out of each gene, two sgRNA sequences were used to knock-out Ikaros (sgIKZF1-1, sgIKZF1-2), Aiolos (sgIKZF3-1, sgIKZF3-2), and ZFP91 (sgZFP91-1, sgZFP91-3). For controls, two non-targeting sgRNAs (sgNT-1, sgNT-2), a sgRNA targeting a non-coding region of the genome (sgNC-1), and a sgRNA targeting an established essential gene ETF1 (sgETF1-1) were included. Following transduction with sgRNAs, cells were washed and mixed at a 1:1 ratio and the percentage of GFP⁺ and RFP⁺ cells was measured every three days for eighteen days (FIG. 7, panel B). A decrease in the RFP⁺/GFP⁺ ratio over time indicates a reduction of cell fitness due to knock-out of the gene targeted by the sgRNA in the RFP-reporter containing construct. As expected, in all six DLBCL lines, knock-out of the essential gene ETF1 resulted in robust depletion of the RFP⁺/GFP⁺ ratio (FIG. 7, panel B). The second non-targeting control (sgNT-2) behaved extremely similarly to the first non-targeting control (sgNT-1) and displayed no change in the RFP⁺/GFP⁺ ratio. Additionally, in all six DLBCL lines transduced with sgRNA targeting a non-coding region of the genome (sgNC-1), a slight reduction in cell fitness was observed, presumably due to the induction of a double stranded break in the DNA and leading to G2 cell cycle arrest.

Knock-out of Ikaros, Aiolos, and ZFP91 resulted in diverse RFP⁺/GFP⁺ ratio depletion across the panel of DLBCL cell lines, indicating highly varying degrees of essentiality for each gene in the different cell lines. Additionally, single gene knock-out of Ikaros, Aiolos, or ZFP91 did not result in the same extent of reduction in the RFP⁺/GFP⁺ ratio as was observed with ETF1 knock-out in any of the cell lines. In some cell lines, the depletion in the RFP⁺/GFP⁺ ratio observed from knock-out of Ikaros, Aiolos, or ZFP91 was similar to that observed with non-coding sgRNA (sgNC-1), suggesting that gene knock-out had a similar degree of inhibition on cell fitness as the induction of the DNA damage response. Gene knockout in each line was confirmed by immunoblot analysis, and revealed that Cas9 expressing DLBCL cells had specific knockout for the indicated target genes in each of the KARPAS-422-Cas9, U-2932-Cas9, RIVA-Cas9, SU-DHL-16-Cas9, HT-Cas9, and SU-DHL-4-Cas9 cell lines expressing guide RNAs for sgNT-1, sgNT-2, sgNC-1, sgIKZF1-1, sgIKZF1-2, sgIKZF3-1, sgIKZF3-2, sgZFP91-1, sgZFP91-3, and sgETF1-1 cells.

Altogether, these data indicate that loss of a single cereblon substrate does not fully account for the cytotoxic effect of Compound 1, and that the antiproliferative effect may be due to the concerted loss of multiple Compound 1-targeted cereblon substrates. Therefore, these results demonstrate that evaluation of more than one cereblon substrate can be used as a response to Compound 1.

### Example 11: Loss of Ikaros or Aiolos Sensitized Diffuse Large B-cell Lymphoma Cell Lines to Compound 1

To explore whether loss of multiple Compound 1 targeted cereblon substrates can cooperate together to elicit enhanced inhibition of cell fitness, individual substrates were knocked-out and concurrently treated with increasing concentrations of Compound 1. If cooperation between substrates exists in mediating the antiproliferative effect of Compound 1, advanced loss of any single substrate involved should render the cells more sensitive to the compound. A similar flow cytometry-based cellular competition assay as presented in FIG. 7, Panel A was utilized to evaluate enhanced sensitivity to Compound 1 upon substrate gene knock-out Increased sensitivity to Compound 1 due to the accelerated loss of a single substrate via gene knock-out was indicated by an enhanced decrease in the RFP⁺/GFP⁺ ratio of gene knock-out cells upon treatment with Compound 1 compared to DMSO.

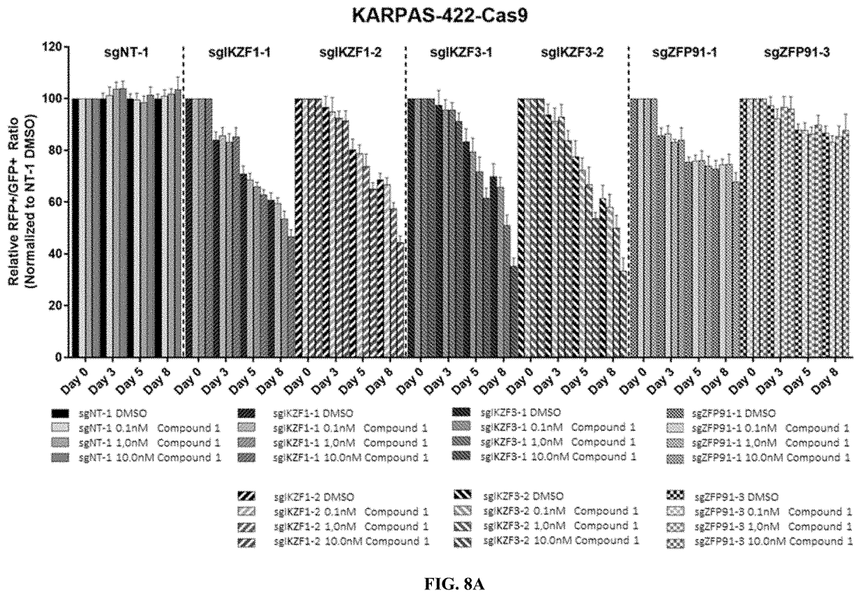

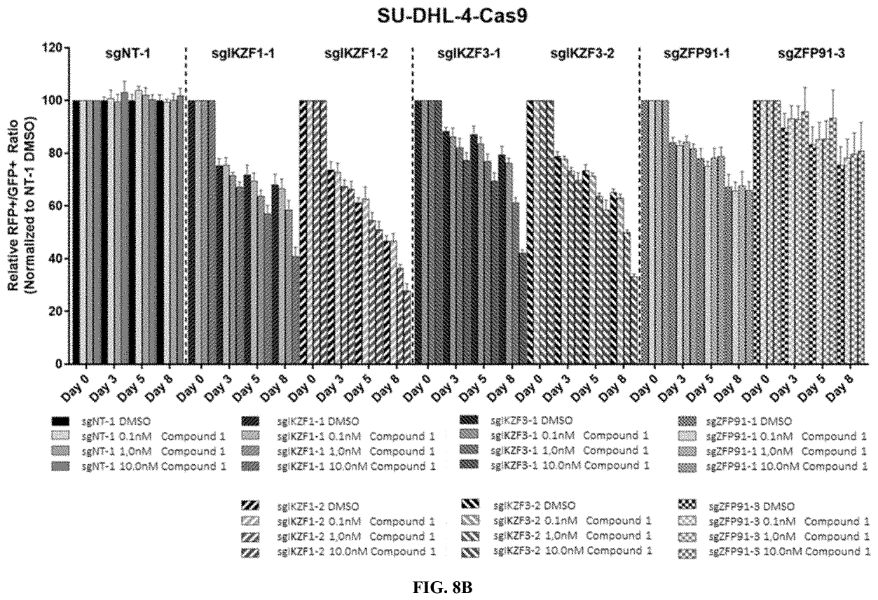

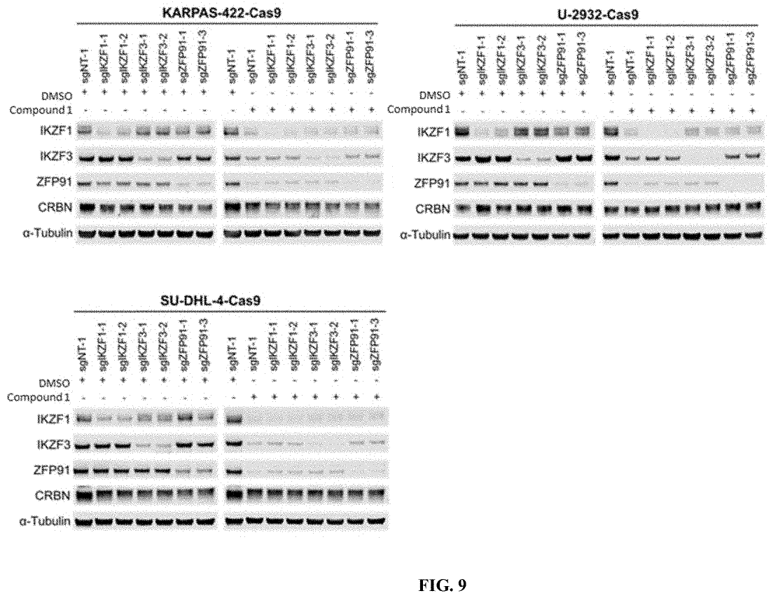

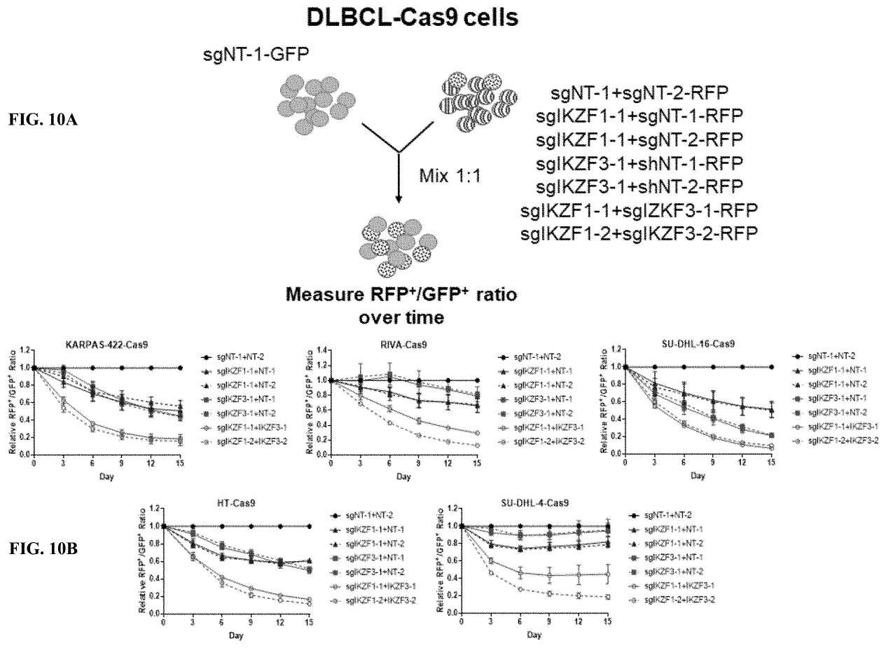

As expected, in control KARPAS-422 (FIG. 8, Panel A), and SU-DHL-4 (FIG. 8, Panel B) sgNT-1 cells (far left group), there was little change in the RFP⁺/GFP⁺ ratio with the addition of 0.1 nM, 1 nM, and 10 nM Compound 1 (gray bars) compared to dimethyl sulfoxide (DMSO)-treated cells (far left column in each subgroup). However, in sgIKZF1 (second group from the left; sgIKZF1-1, thin rightward angled lines; and sgIKZF1-2, thick rightward angled lines) and sgIKZF3 (third group from the left; sgIKZF3-1, thin leftward angled lines; and sgIKZF3-2, thick leftward angled lines) knock-out cells treated with Compound 1, there was a dose-dependent decrease in the RFP⁺/GFP⁺ ratio relative to their respective DMSO-treated control cells (far left column in each subgroup). This effect was not observed in sgZFP91 cells (far right group; sgZFP91-1, thin checkered pattern; and sgZFP91-3, thick checkered pattern), suggesting that loss of ZFP91 does not cooperate with the loss of other Compound 1-targeted cereblon substrates to enhance the antiproliferative effects of Compound 1. Additionally, there was an observed decrease in the RFP⁺/GFP⁺ ratio over time for all DMSO-treated sgIKZF1, sgIKZF3, and sgZFP91 cells (far left black columns in each subgroup) knock-out relative to DMSO-treated control sgNT-1 cells (far left black columns in each subgroup), which is the result of decreased cell fitness from knocking out the respective gene and is consistent with the data presented in FIG. 7, Panel B. Furthermore, it was confirmed by immunoblot analysis that 24-hour Compound 1 treatment effectively promoted substrate degradation in substrate knock-out cells (FIG. 9). Therefore, these results indicate that Ikaros, Aiolos, and to a lesser extent ZFP91, can serve as markers of sensitivity to Compound 1.

### Example 12: Combined Ikaros and Aiolos Loss had an Additive Effect on Inhibiting Diffuse Large B-cell Lymphoma Cell Fitness

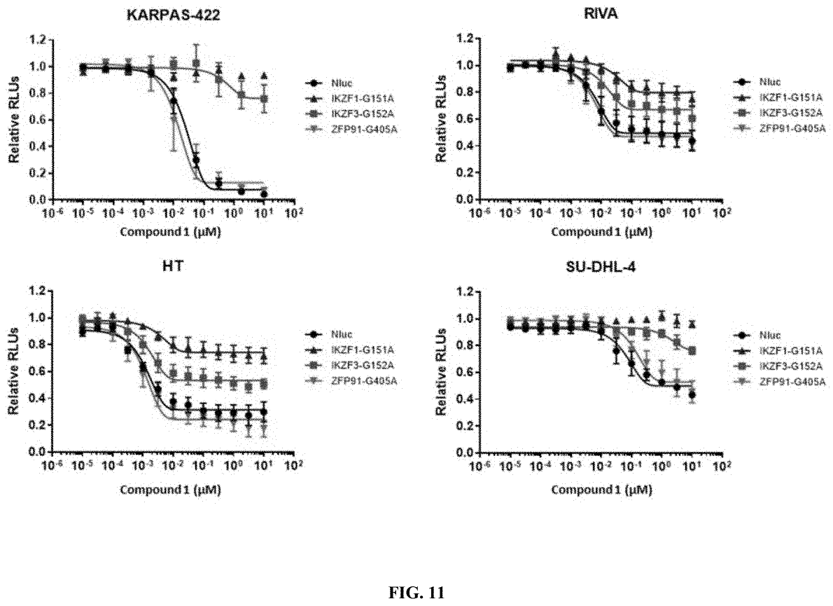

Ikaros and Aiolos are highly homologous transcription factors that can form homodimers or heterodimers. To explore whether Ikaros and Aiolos may possess any redundancy in promoting DLBCL cell survival and proliferation, dual sgRNA-mediated knock-out of Ikaros and Aiolos was performed in six DLBCL cell lines: KARPAS-422, U-2932, RIVA, SU-DHL-16, HT, and SU-DHL-4. A flow cytometry-based competition assay was employed to assess cell fitness, where cells were transduced with sgNT-1-GFP or with constructs expressing two sgRNAs from the same vector with an RFP reporter (FIG. 10, Panel A). The combinations of dual sgRNAs were sgNT-1+sgNT-2, sgIKZF1-1+sgNT-1, sgIKZF1-1+sgNT-2, sgIKZF3-1+sgNT-1, sgIKZF3-1+sgNT-2, sgIKZF1-1+sgIKZF3-1, and sgIKZF1-2+sgIKZF3-2. Once again, after transduction, cells were washed and mixed at a 1:1 ratio and the percentages of GFP⁺ and RFP⁺ cells were measured every three days for fifteen days.

In all six cell lines, the knock-out of both Ikaros and Aiolos elicited a greater decrease in the RFP⁺/GFP⁺ ratio compared to knockout of either Ikaros or Aiolos alone (FIG. 10, Panel B). Specificity for knock-out of Ikaros, Aiolos, or both was confirmed by immunoblot analysis, and revealed that Cas9 expressing DLBCL cells had specific knockout for IKZF1 or IKZF3 in cell lines that contained the relevant gRNA for the gene(s) in each of the KARPAS-422-Cas9, U-2932-Cas9, RIVA-Cas9, SU-DHL-16-Cas9, HT-Cas9, and SU-DHL-4-Cas9 cell lines expressing dual guide RNAs for sgIKZF1-1+sgNT-1, sgIKZF1-1+sgNT-2, sgIKZF3-1+sgNT-1, sgIKZF3-1+sgNT-2, sgIKZF1-1+sgIKZF3-1, and sgIKZF1-2+sgIKZF3-2, whereas sgNT-1+sgNT-2 showed no change in IKZF1 or IKZF3 protein levels.

These results indicated that Ikaros and Aiolos possess some redundant functions in DLBCL cell lines, and that loss or degradation of both Ikaros and Aiolos can serve as a marker of response to Compound 1.

### Example 13: Inhibition of Ikaros or Aiolos Degradation Protected Diffuse Large B-Cell Lymphoma Cells from Compound 1

To test whether Ikaros and Aiolos were redundant in promoting the survival and proliferation of DLBCL lines, degradation-resistant mutants of Ikaros (IKZF1-G151A), Aiolos (IKZF3-G152A), and ZFP91 (ZFP91-G405A), along with NanoLuc luciferase (Nluc) as a control, were ectopically and stably expressed in four DLBCL cell lines: KARPAS-422, RIVA, HT, and SU-DHL-4. Dose response curves with Compound 1 indicate that expression of degradation-resistant mutants of Ikaros or Aiolos provided protection from Compound 1 in all four cell lines (FIG. 11). Additionally, the ectopic expression of the degradation-resistant mutants, as well as their protection against Compound 1-induced degradation, was confirmed by immunoblot analysis. For example, immunoblot analysis showed that Ikaros, Aiolos, and ZFP91 protein levels were not substantially affected by Compound 1 treatment in KARPAS-422, RIVA, HT, and SU-DHL-4 cell lines ectopically expressing the degradation-resistant mutants of Ikaros (IKZF1-G151A), Aiolos (IKZF3-G152A), or ZFP91 (ZFP91-G405A), relative to the same cell lines ectopically expressing NLuc. For example, treatment of NLuc expressing cells with 5 nM or 50 nM of Compound 1 resulted in an appreciable decrease in IKZF1, IKZF3, and ZFP91 protein levels, as compared to control or DMSO treated cells. Beta-actin levels were used as a control. Ectopic expression of IKZF1-G151A in the four cell lines revealed that IKZF1 protein levels were not decreased in response to 5 nM or 50 nM of Compound 1 treatment for 24 hours, but the levels of IKZF3 and ZFP91 were still decreased in response to Compound 1 treatment Similar patterns were observed with IKZF3-G152A, and ZFP91-G405A. However, there were varying degrees of protection observed in the four cell lines. In KARPAS-422, and SU-DHL-4, a nearly complete protection against Compound 1 was observed, suggesting that Ikaros and Aiolos possess truly redundant functions in these lines. In HT and RIVA, however, the protection was not as robust Taken together, these data highlight the usefulness of Ikaros, Aiolos, or both in response to Compound 1.

### Example 14: Compound 1 Promoted the Degradation of Ikaros in T Cells without Significantly Affecting their Viability

The immunomodulatory activity of Compound 1 on ex vivo stimulated PBMCs was assessed. Disease progression in lymphoma patients has been associated with impaired immune system function. Exhausted T cells show reduced differentiation, proliferation and function in cytokine production.

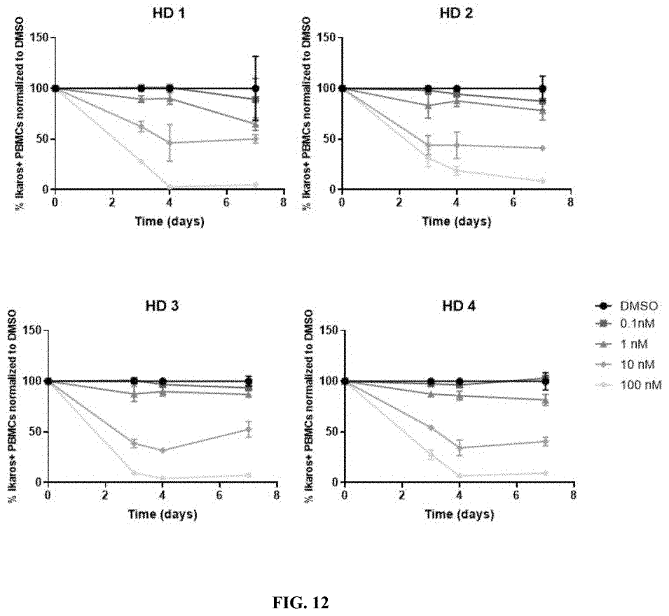

Purified PBMCs from four healthy donors were plated on anti-CD3-coated plates to stimulate T cells and subsequently treated with either DMSO (control) or Compound 1 at various concentrations. The ability of Compound 1 to promote the degradation of Ikaros was assessed over time by flow cytometry (FIG. 12). Compound 1 induced concentration-dependent degradation of Ikaros in PBMCs evidenced by a decrease in the percentage of cells that were positive for Ikaros over time. Moreover, degradation was maintained up to 7 days after just a single treatment of Compound 1.

To establish that this was due to the effect of the compound in promoting Ikaros degradation and not because of a lack of cell proliferation or viability of the CD3-stimulated PBMCs, the effect of Compound 1 on their viability was assessed by flow cytometry. Cells treated with Compound 1 did not show a significant effect on PBMC viability, which was comparable to that of control DMSO-treated cells over time. Peripheral blood mononuclear cells derived from four healthy donors were exposed to Compound 1 at concentrations of 0.1, 1, 10, or 100 nM for 3, 4, or 7 days, and viability was measured by staining with Viobility™ Fixable Dye. The results indicated that on day 3 and day 4, roughly 80% of the PBMCs were viable, and Compound 1 had no significant effect at any of the concentrations. The trend in cell viability continued through day 7, with Compound 1 treatment not effecting the viability of the PBMCs.

These results demonstrate that the expression levels of Ikaros in T cells can be degraded by Compound 1, and that Ikaros levels can serve as a marker of response to Compound 1 in T cells.

### Example 15: Effect of Compound 1 on Effector Lymphocytes and Cytokine Production

The results from Example 14 demonstrated that Compound 1 promotes Ikaros degradation, and Ikaros is known to act as a repressor of IL-2 expression and secretion, a marker of T-cell activation. Accordingly, the effect of Compound 1 on effector T cell cytokine secretion was assessed.

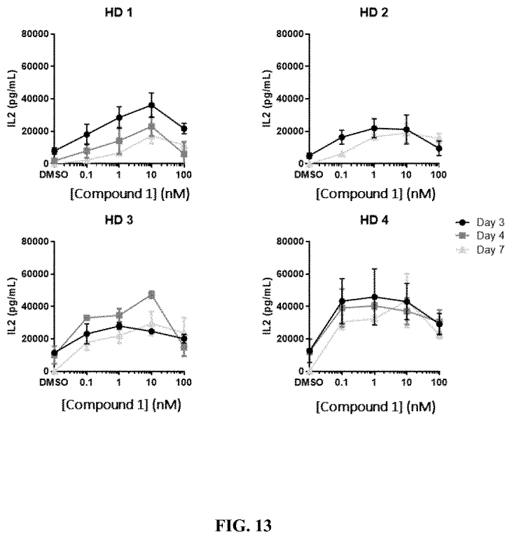

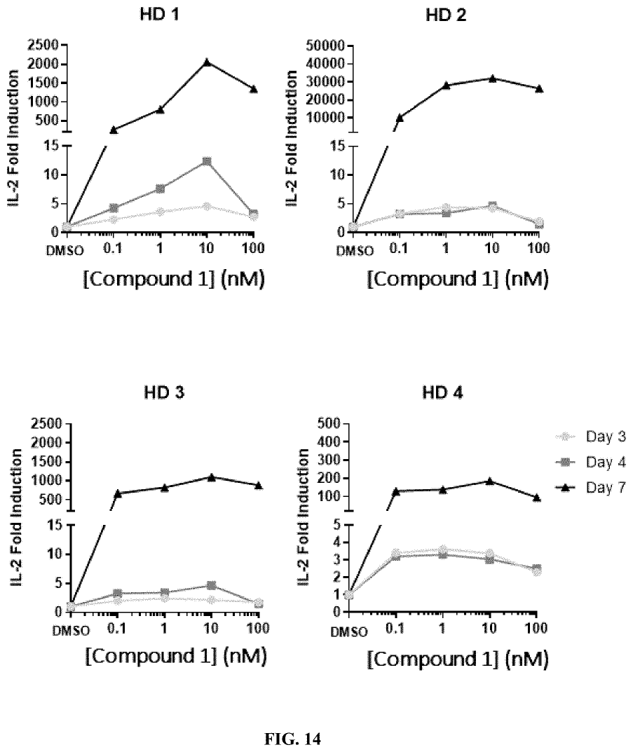

Purified PBMCs from four healthy donors were plated on anti-CD3 antibody-coated plates to stimulate T cells and were subsequently treated with either DMSO (control) or Compound 1 at various concentrations for 3, 4, and 7 days. Secretion of cytokines was measured in supernatant fluid from PBMC cultures over time by using mesoscale (MSD) assays. Interleukin-2 secretion by PBMCs during in vitro culture was increased upon exposure to Compound 1 as shown in FIG. 13 and FIG. 14. Compound 1 induced IL-2 secretion at all concentrations tested. This activity in IL-2 occurred at concentrations of Compound 1 (0.1 to 100 nM) that have been shown to produce strong antiproliferative activity against DLBCL tumor cells. Transient or no effect was observed with other monitored cytokines (TNFα, IFNγ, IL-4, IL-13, IL-10, and IL-6). This demonstrated that IL-2 secretion can serve as a marker of response to Compound 1, particularly for T-cell activation.

### Example 16: Compound 1 Induced the Secretion of Cytokine/Chemokines in Exhausted T Cells

Exhausted T cells differ phenotypically from functional effector T cells as they acquire the expression of inhibitory signaling pathways including the programmed cell death protein 1 (PD1) and the lymphocyte activation gene 3 protein (LAG3). Exhausted T cells show reduced differentiation, proliferation, and production of cytokines.

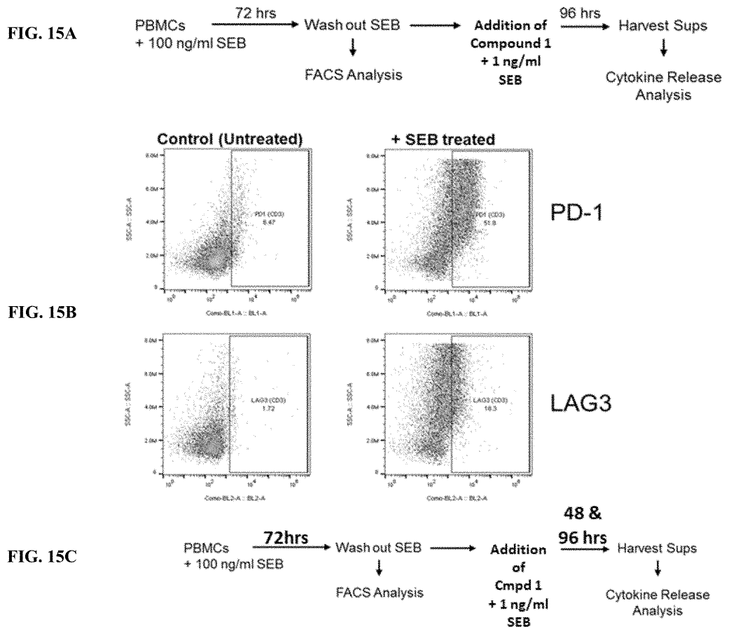

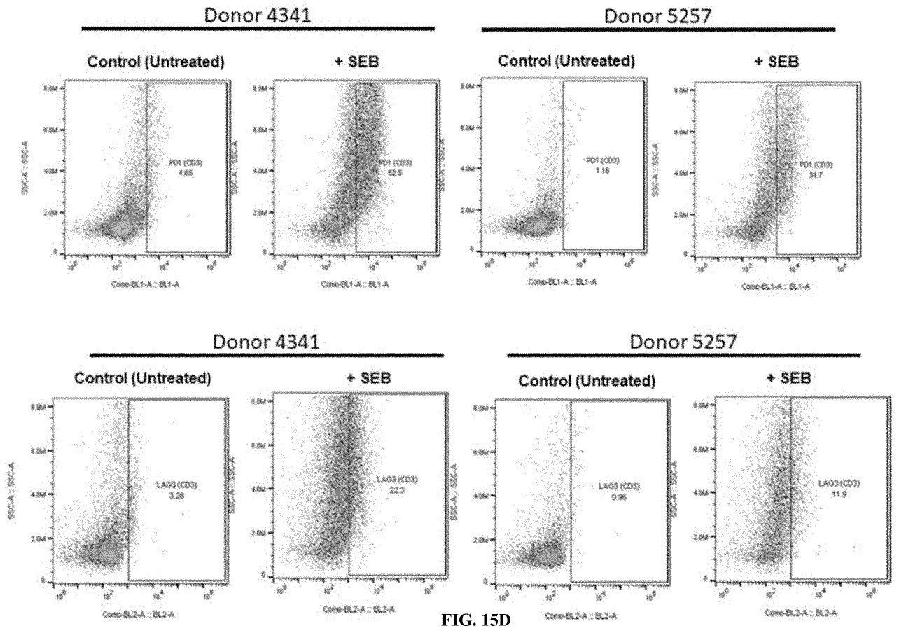

To induce T cell exhaustion, PBMCs from three donors were treated for 72 hours with 100 ng/mL staphylococcal enterotoxin B (SEB) (FIG. 15, Panel A and panel C). On Day 3, the SEB was washed out and the expression of the exhaustion markers PD1 and LAG3 of the CD3− positive T cells was assayed by FACS (FIG. 15, Panel B). The exhausted T cells of one donor were then subsequently treated with Compound 1 for 96 hours and exhausted T cells of two additional donors were treated with Compound 1 for 48 and 96 hours in the presence of 1 ng/mL SEB and the release of effector cytokines into the culture medium was assessed by MSD analysis. The results indicated that Compound 1 increased the secretion levels of granulocyte macrophage colony stimulating factor (GM-CSF), interferon gamma (IFNγ), and tumor necrosis factor alpha (TNFα) in a concentration and time dependent manner, as measured by mesoscale (MSD) analysis after 48 hours or 96 hours. For example, approximately 0.001 μM of Compound 1 for 96 hours resulted in 3000 pg/mL of GM-CSF, whereas approximately 0.01 μM of Compound 1 for 96 hours resulted in 10,000 pg/mL of GM-CS, and the amount secreted continued to increase until it plateaued just under 15,000 pg/mL at concentrations greater than 0.1 μM of Compound 1. Exposure to Compound 1 for 96 hours indicated that 96 hours of treatment with Compound 1 led to higher amount of GM-CSF and TNFα release, and to a lesser extent IFNγ, relative to exposure for 48 hours.

Compound 1 induced the effector cytokines/chemokines GM-CSF, TNFα and IFNγ secretion in the T cell restimulation assay. Compound 1 also showed activity with inducing the secretion of the three effector cytokines/chemokine at concentrations in the range of 0.01 to 10 μM and in the three donor PBMCs evaluated. The EC₅₀ values achieved for secreted GM-CSF levels (EC₅₀=0.006 μM), TNFα (EC₅₀=0.01 μM), and IFNγ (EC₅₀=0.01 μM) supports that the immunomodulatory activity of Compound 1 occurs at concentrations with strong antitumor effect in vitro.

Taken together, these results demonstrated that Compound 1 has immunomodulatory activity. Notably, the immunomodulatory activity occurs at similar concentrations that lead to antitumor effects in the panel of DLBCL cell lines. Furthermore, these results demonstrate that secretion of the effector cytokines/chemokines GM-CSF, TNFα and IFNγ from T-cells can serve as a marker of response to Compound 1, and that expression of PD-1 and LAG3 can be used as markers of identifying cells that might respond to Compound 1 treatment.

### Example 17: Compound 1 and its R-Enantiomer Exhibited Similar Antiproliferative Activity and Bind Cereblon

To evaluate the activity of both Compound 1 and Compound 2 in DLBCL cells, a CellTiter-Glo (CTG) assay was performed to measure the antiproliferative activity. Potent antiproliferative activity in the DLBCL cell line SU-DHL-4 was observed for both Compounds 2 and 3 (Table 8).

TABLE 8
Antiproliferative Potency of Enantiomers in Diffuse
LargeB-Cell Lymphoma Cells Assessed in a 5-day Assay

| | Antiproliferative Activity | |
|---|---|---|
| | IC₅₀ (μM) | |
| | Compound 1 | Compound 2 |
| DLBCL Cell Line | (Senantiomer) | (Renantiomer) |
| SU-DHL-4 | 0.022 (n = 6) | 0.032 (n = 6) |

DLBCL = diffuse large B-cell lymphoma;
IC₅₀ = 50% inhibitory concentration.

To investigate whether Compound 1 and its R-enantiomer Compound 2 could bind to cereblon, a short duration (10 to 20 minutes) ligand competition assay was used. Samples of Compound 1 and Compound 2 with greater than 99% chiral purity were evaluated for cereblon binding affinity in a TR-fluorescence resonance energy transfer (FRET) assay (Table 8). The results revealed that both Compound 1 and Compound 2 bind to CRBN in a concentration-dependent manner. Specifically, Compound 1, had an eleven-fold higher binding affinity for CRBN binding (IC50=0.86±0.1 μM, n=5) compared to Compound 2 (IC50=10.25±1 μM, n=5).

Taken together, these results indicate that both Compound 1 and Compound 2 display activity in DLBCL cells, and that Cereblon can serve as a marker of response.

### Example 18: Compound 1 and Compound 2 Induces Degradation of Aiolos, Ikaros, and ZFP91

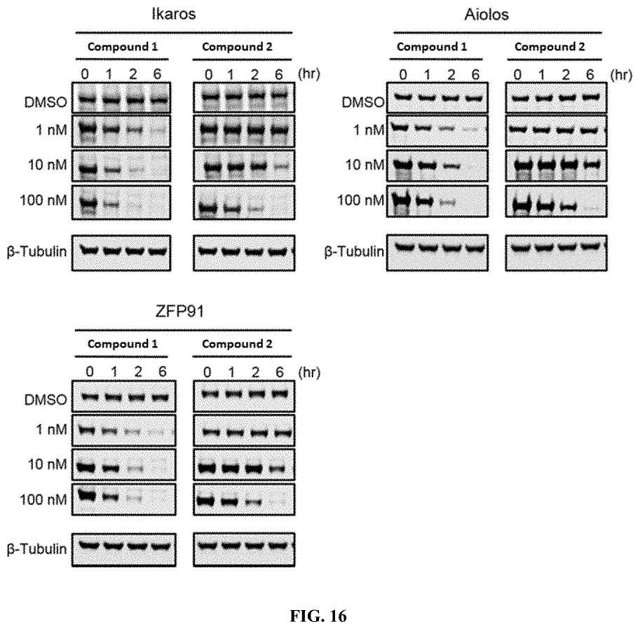

Pure samples of each enantiomer were tested for cellular degradation of substrate proteins measured over a short time-course (FIG. 16). SU-DHL-2 cells were treated for 1, 2, and 6 hours with vehicle control (0.1% DMSO), Compound 1 (1, 10, 100 nM), or Compound 2 (1, 10, 100 nM). Western blot analysis was used to evaluate the extent of degradation of the protein substrates Aiolos, Ikaros, and ZFP91. Compound 1 inhibited Aiolos, Ikaros, and ZFP91 in as early as 1 hour of treatment with as little as 1 nM. More than 50% degradation of each of the three proteins occurred within 2 hours at 1 nM Compound 1, and all three proteins showed similar rates of degradation. Compound 2 also led to decreased expression of Aiolos, Ikaros, and ZFP91, although with different kinetics (FIG. 16). For example, decreased expression of Aiolos, Ikaros, and ZFP91 was observed at higher concentrations and a longer period of exposure to Compound 2.

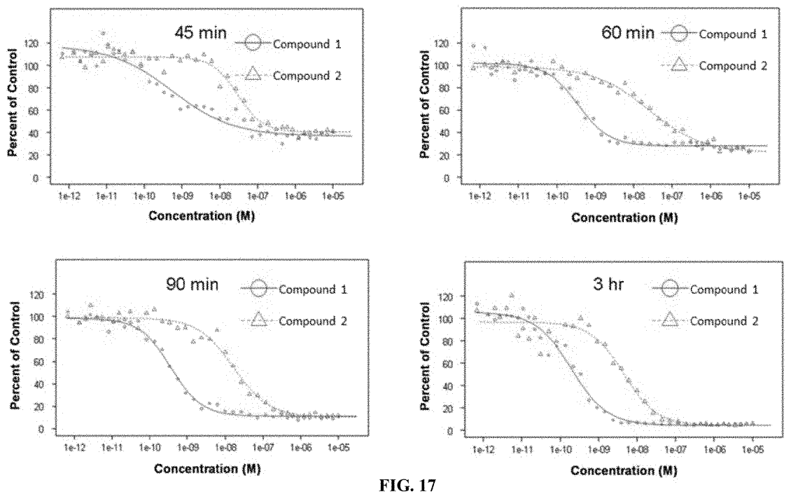

In a second assay, DF15 cell lines expressing ePL-tagged target protein substrates were used as a model system to monitor protein degradation at 0.75, 1, 1.5, 3, 4, and 24 hours over a concentration range from 1 pM to 10 μM. The proteins evaluated were Ikaros (FIG. 17), Aiolos, and ZFP91. Compound 1 and Compound 2 were both able to degrade the substrate proteins at all time points (Table 9). In a further assay, additional time points were included to monitor the effect of treatment with Compound 1 and Compound 2. The results confirm that Compound 1 and Compound 2 degraded Aiolos, Ikaros, and ZFP91 in a concentration and time dependent manner in DF-15 cells expressing Enhanced ProLabel (ePL)-Aiolos, ePL-Ikaros, or ePL-ZFP91 after exposure for 1, 2, 6, or 24 hours, with the cells being more sensitive to Compound 1, as compared to Compound 2.

The depth of substrate protein degradation described by the Y-constant was also measured in this assay against a negative control luciferase inhibitor (CC1071297, Y-constant=0). Both enantiomers showed similar Y-constant values (Table 9), which corresponds to similar lower Y asymptotes in the drug response curves (FIG. 17).

TABLE 9
Comparison of the Kinetics of Substrate Degradation Induced
by Compound 1 and its R-enantiomer in DF15 Cells

| | Time | | | | |
|---|---|---|---|---|---|
| Substrate | point | Compound 1 | | Compound 2 | |
| Protein | (hours) | EC₅₀ (nM) | Y-constant | EC₅₀ (nM) | Y-constant |

Ikaros

| | | | | |
|---|---|---|---|---|
| 0.75 | 1.25 | 39.3 | 39.7 | 47.0 |
| 1 | 1.01 | 27.5 | 27.0 | 34.4 |
| 1.5 | 0.96 | 12.5 | 22.5 | 15.3 |
| 3 | 0.81 | 8.6 | 8.88 | 7.3 |
| 4 | 0.99 | 6.8 | 7.31 | 5.5 |
| 24 | 0.35 | 2.6 | 0.70 | 2.4 |

Aiolos

| | | | | |
|---|---|---|---|---|
| 0.75 | 0.73 | 46 | 38 | 54 |
| 1 | 0.94 | 29 | 24 | 37 |
| 1.5 | 0.72 | 11 | 17 | 15 |
| 3 | 0.60 | 4.8 | 10 | 3.5 |
| 4 | 0.56 | 3.9 | 7.3 | 3.5 |
| 24 | 0.52 | 4.0 | 5.3 | 3.8 |

ZFP91

| | | | | |
|---|---|---|---|---|
| 0.75 | 0.54 | 37 | 29 | 41 |
| 1 | 0.36 | 28 | 25 | 22 |
| 1.5 | 0.36 | 11 | 16 | 10 |
| 3 | 0.19 | 4.9 | 4.5 | 4.6 |
| 4 | 0.17 | 4.3 | 2.6 | 4.1 |
| 24 | 0.15 | 2.0 | 0.35 | 2.5 |

EC₅₀ = half-maximal effective concentration;
ZFP91 = zinc finger protein 91.

Taken together, these results demonstrate that Ikaros, Aiolos, and ZFP91 levels can serve as markers of response to both Compound 1 and Compound 2.

### Example 19: Effect of Compound 1 on Maturation of Neutrophil Precursors

The effects of Compound 1 on maturation of myeloid progenitors to neutrophils were assessed in ex vivo cultures of bone marrow CD34+ cells from healthy donors and different dosing schedules were evaluated to gain insight into the schedule dependence of these events.

Myeloid differentiation was induced by adding stem cell factor (SCF), FMS-related tyrosine kinase 3 ligand (FLT3-L), and granulocyte colony stimulating factor (G-CSF) to culture media. Cell differentiation in the presence or absence of Compound 1 was evaluated at prespecified time points by flow cytometry as the percentage of cells in 5 subpopulations: (1) hematopoietic stem cells (HSC, CD34+/CD33−/CD11b−); (2) Stage I cells (CD34+/CD33+/CD11b−); (3) Stage II cells (CD34−/CD33+/CD11b−); (4) Stage III cells (CD34−/CD33+/CD11b+) and (5) Stage IV cells (CD34−/CD33−/CD11b+) cells (from immature to mature). Differentiation and viability were monitored during the complete assay every two or three days.

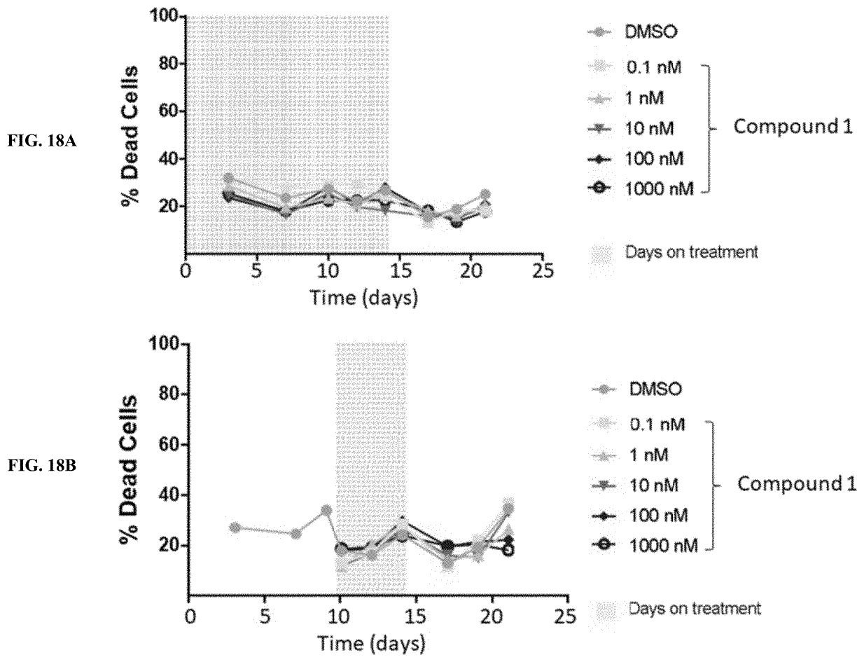

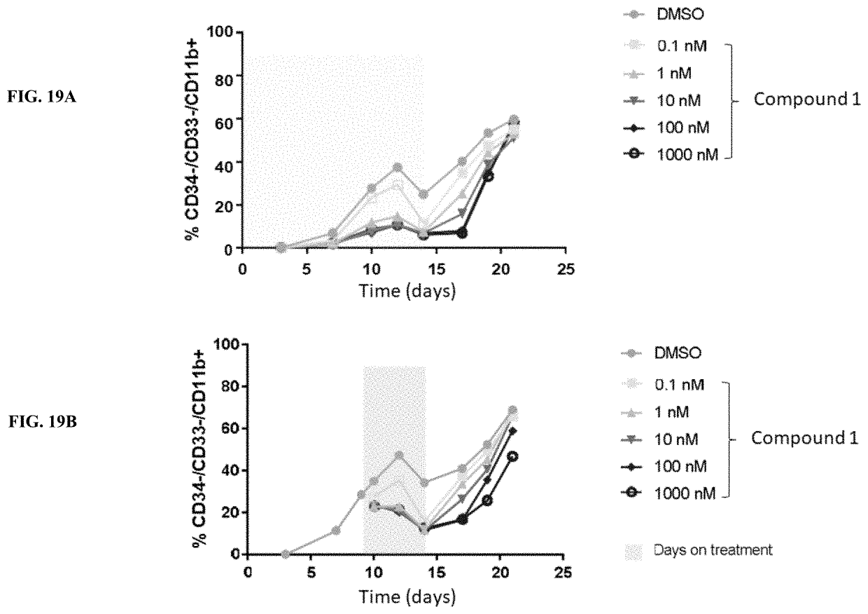

The effects of different Compound 1 exposure periods (14 or 5 days on treatment) on viability and maturation of myeloid progenitors to neutrophils were evaluated at concentrations of 1, 10, and 100 nM of Compound 1 at the indicated times (FIG. 18; FIG. 19) for up to 21 consecutive days using flow cytometry. Results showed that late-stage maturation of neutrophil progenitors was blocked by Compound 1, with mature neutrophils significantly reduced in number at the higher concentrations after 14 (FIG. 19A) or 5 days (FIG. 19B) of exposure. Maturational arrest appears to occur primarily at Stage III neutrophil progenitor development, as evidenced by an accumulation of cells with Stage III cell surface immunophenotype and a reduction in the population of cells with Stage IV cell surface immunophenotype (mature neutrophils). However, the viability of neutrophil precursors exposed to Compound 1 was not affected (FIG. 18).

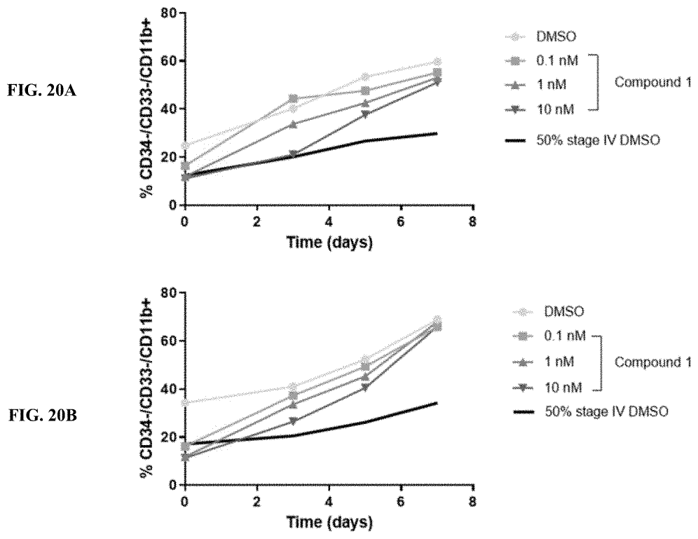

Recovery of mature neutrophils after Compound 1 exposure was also evaluated in the system. After a period of 1 week without Compound 1, the proportion of mature neutrophils (Stage IV cells) recovered by at least ˜50% from its nadir, with a trend towards more rapid and complete recovery at lower concentrations (FIG. 20; Table 10).

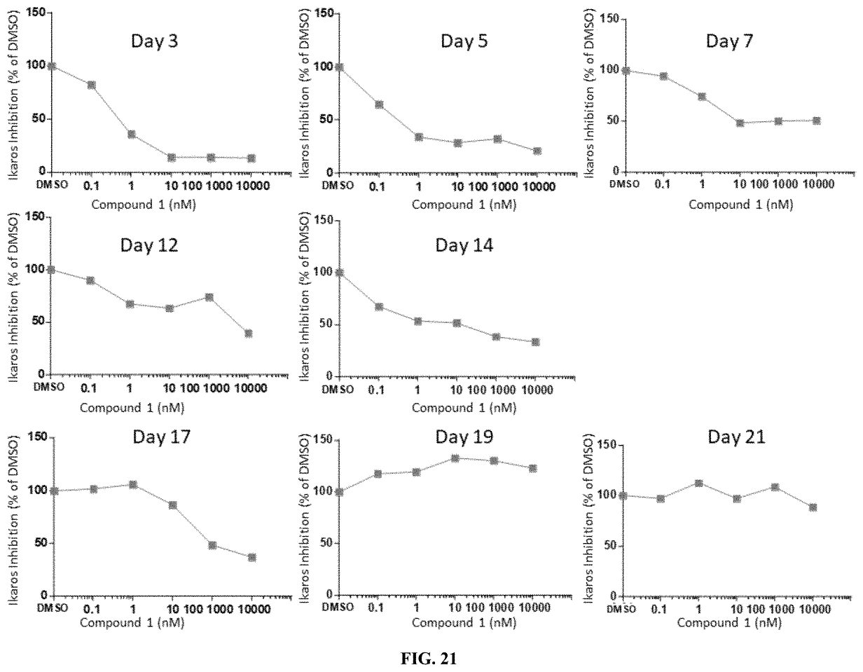

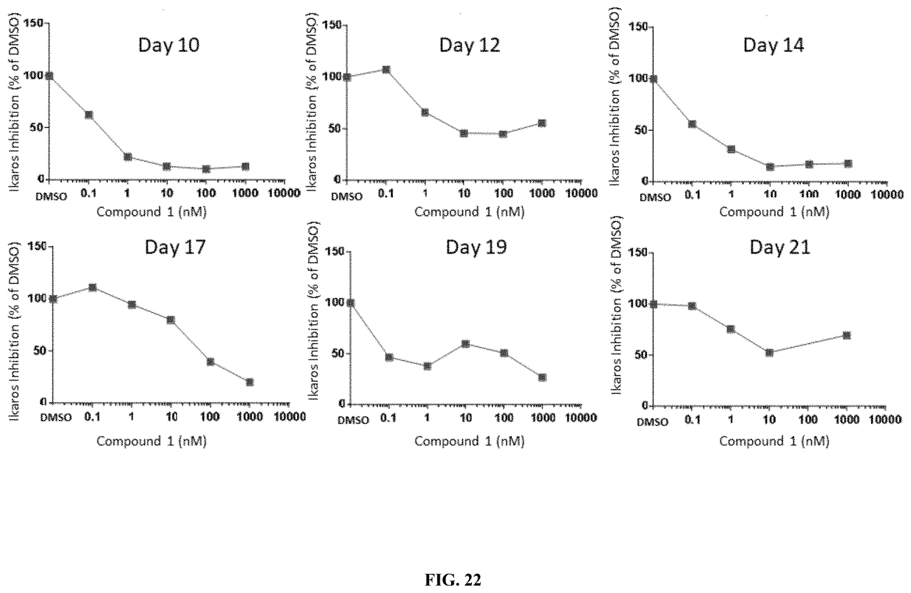

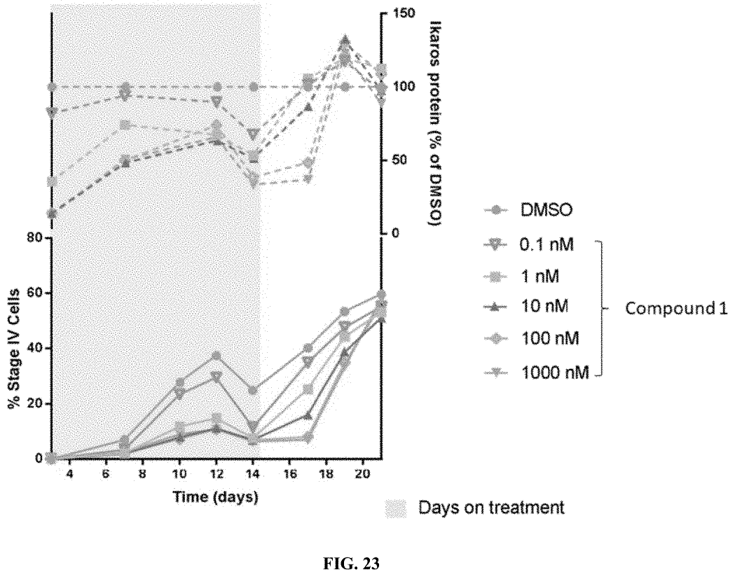

Ikaros protein levels were monitored during periods of Compound 1 exposure and recovery. Ikaros levels were reduced during Compound 1 exposure and recovered following drug withdrawal in a concentration-dependent manner with no significant differences noted in association with different exposure schedules (FIG. 21; FIG. 22). Ikaros levels began returning to normal after at least 3 days following washout, predating full recovery of maturation of late-stage neutrophil precursors (FIG. 23). These results demonstrate that Ikaros degradation in late stage neutrophil precursors could be an important mediator of neutropenia in recipients of Compound 1. Furthermore, the findings suggest that restoration of Ikaros levels precedes recovery of maturation of neutrophil progenitors. Accordingly, Ikaros levels in neutrophils can serve as a marker for response to Compound 1.

Recovery of mature neutrophil levels to at least 50% of the untreated control level in the in vitro assay system utilized in the present study correlates to the absence of clinically significant neutropenia in patients. In the current study, after a period of one week without drug, the population of Stage IV cells were able to recover in a concentration dependent manner to a level equal to at least 50% that of the Stage IV cell population in the DMSO control (FIG. 20) under all tested conditions evaluated and even reach DMSO control levels under some of the conditions tested.

TABLE 10
Amount of Time Needed for Recovery to
50% Stage IV Cells after a Continuous
Exposure to Compound 1 for 14 Days
or 5 Days with a One-week Washout after Treatment

| | Number of Days in Washout Period Required | | | | |
|---|---|---|---|---|---|
| Concentration of | for Recovery to 50% of Stage IV Cells | | | | |
| Compound 1 | 0.1 nM | 1 nM | 10 nM | 100 nM | 1000 nM |
| 14 days treatment | 0 | 0 | 0 to 2 | 4 | 4 |
| 5 days treatment | 0 | 1 | 1 | 3 to 4 | 5 |

Ikaros protein level was analyzed by flow cytometry every two or three days. As shown in FIG. 21 and FIG. 22, Ikaros protein was degraded under both Compound 1 treatment schedules (14 days and 5 days) and its expression was restored after drug washout in a concentration dependent manner. At Day 19, a complete recovery of Ikaros was observed at all concentrations after 14 days of treatment Recovery of Ikaros protein expression was slower in cells treated for 5 days than in cells treated for 14 days: at Day 19, recovery of Ikaros protein was not complete at any concentration of Compound 1 and, at Day 21, the level of Ikaros protein had fully recovered in cultures exposed to 10 nM Compound 1 only.

In the current study, as shown FIG. 23 in cells exposed to Compound 1 for 14 days, Ikaros control levels were restored before recovery of differentiation of neutrophil precursors. At concentrations of 10 to 1000 nM Compound 1, recovery of maturation of neutrophil precursors occurred more slowly than recovery of Ikaros protein levels.

Taken together, these results demonstrate that Ikaros protein levels in myeloid cell, as well as apoptosis and myeloid differentiation in CD34+ cells can serve as markers of response to Compound 1.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

### Example 20: Compound 1 Response Markers Identified from Integrated Analysis of Transcriptomic and Proteomic Profiling

Biomarkers associated with the cytotoxic effects of Compound 1 treatment in DLBCL, such as signaling and regulatory pathways, were determined by integrative network analysis using transcriptomic and proteomic profiling. The approach consisted of a network flow optimization using a knowledge driven backbone network with protein interactions and regulatory associations similar to what was described previously (Basha, Mauer, Simonovsky, Shpringer, & Yeger-Lotem, 2019; Gosline, Spencer, Ursu, & Fraenkel, 2012).

Briefly, differential expression analysis and differential protein abundance analysis at different timepoints were integrated using a model framework. This network is a tri-partite graph that includes protein-protein interaction (PPi) network layers, transcriptional regulatory network layers and pathway definitions compiled from the following sources:
Transcriptional Regulatory Network (TRN)
   In-house IKZF3 & ZFP91 gene signatures and ReMAP (492 TFs) (Cheneby, Gheorghe, Artufel, Mathelier, & Ballester, 2018)
   ChIP-Seq peaks annotated to transcriptional start sites (TSS), and filtered by proximity (TSS±2 kbp)
   TRN based on 436 transcription factors and 21,633 genes (regulon sizes E [10,7500])
Protein-Protein interaction Network (PPiN)
   Integration of HINT (Das & Yu, 2012), H-II-14 (Rolland et al., 2014), Human soluble protein complexes (Ruepp et al., 2010) & BioPlex (Huttlin et al., 2015) and filtering by high-confidence experimental evidences
   13872 proteins×144344 edges (after taking account for directionality)
Pathways from MSIgDB-C2 v6.1
   8.904 genes×1,329 pathways (Subramanian et al., 2005)

Edge capacity was weighted by the harmonic mean of it-scores (Xiao et al., 2014) as the standardized log₂ fold change from the differential expression and protein abundance analysis regularized by log₁₀ p-values for each pair of interaction partners. Scores were standardized and scaled between −1 and 1.

Flux calculation was done using maximum-flow method as implemented in Bioconductor's graph.maxflow package. An iterative process was implemented to filter out the least relevant interactions while maintaining a significant amount of the flux running through the network. Edges with a flux lower than the 5th quantile were removed at each iteration, and paths disconnected from the source/sink were pruned until the optimization criteria was satisfied.

To begin, cell line sensitivity to Compound 1 was determined by flow cytometry for 40 cell line models using DRAQ7 and Annexin V staining to quantify the reduction in proliferation and induction of apoptosis after five days of compound treatment. Dose dependency curves were drawn and area under the curves (AUC) were computed for each cell line. Sensitivity was calculated by calculating the ratio of (AUCₐₚₒₚₜₒₛᵢₛ/AUC_{live cells}), where smaller ratios indicated resistance, and larger ratios indicated sensitivity. A panel of 11 DLBCL cell lines were found to have various sensitivities to Compound 1 treatment, ranging from resistant to sensitive.

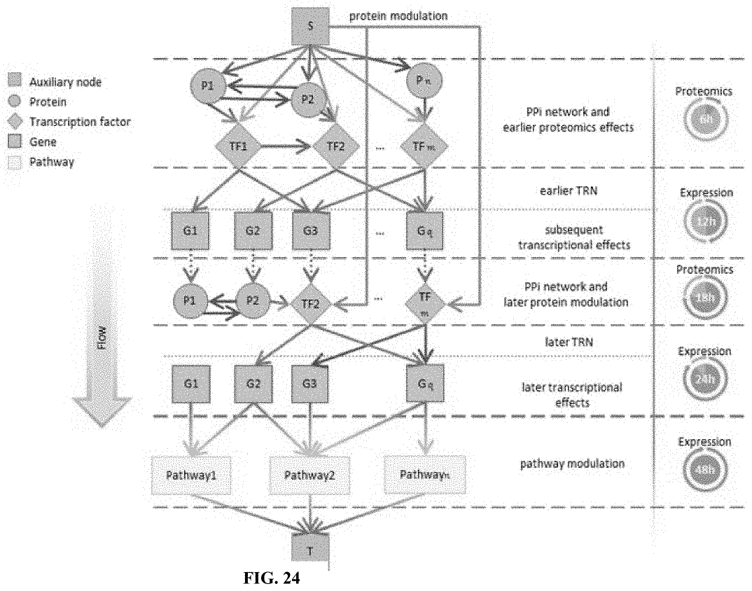

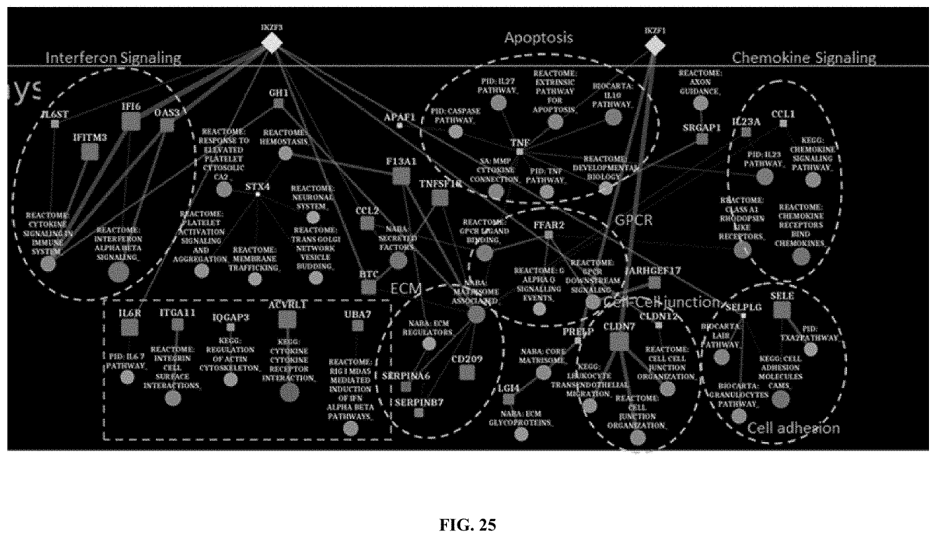

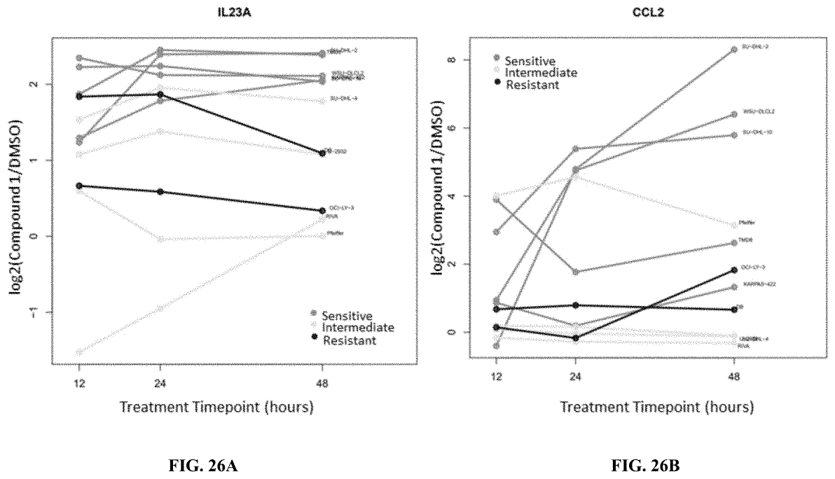

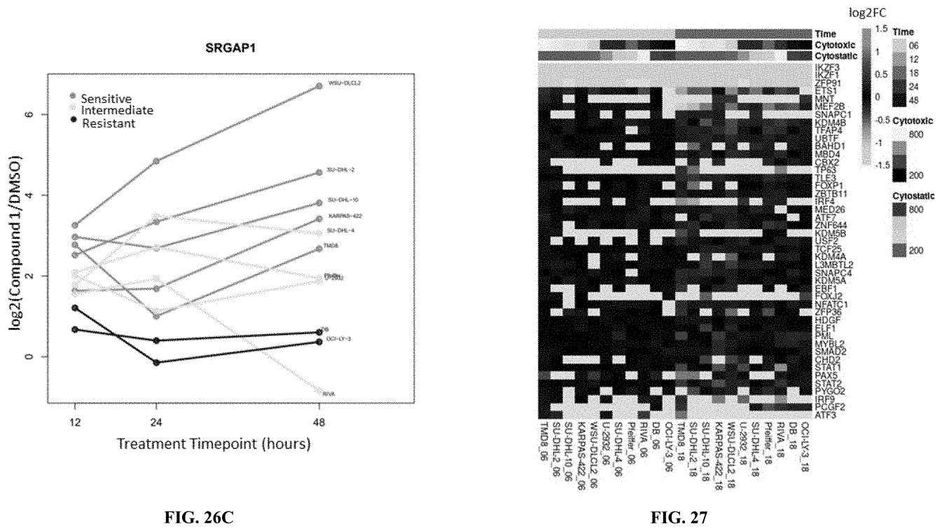

Next, the 11 DLBCL cell lines covering a wide range of the spectrum for sensitivity to Compound 1 were exposed to 0.04 μM of Compound 1 or DMSO controls, and proteomics (TMT-MS) and gene expression (RNA-Seq) profiles were obtained in triplicate for the panel. Samples were profiled at five timepoints after exposure: 6, and 18 hours for proteomics; and 12, 24 and 48 hours for transcriptomics profiles. A scheme of the dynamic integration of the proteomic and transcriptomic information is shown in FIG. 24. The integrated mechanistic analysis revealed that Compound 1 treatment produced an Ikaros/Aiolos-driven up-regulation of genes associated with interferon signaling (e.g., IL6ST, IFITM3, IFI6, OAS3, interferon α/β signaling), cytokine/chemokine signaling (e.g., IL23A, CCL1), apoptosis (e.g., IL27, TNF, IL10, caspase), cell adhesion (e.g., SELE, SELPLG, TXA2), cell-cell junction (e.g., CLDN7, CLDN12), G-protein coupled receptors (e.g., FFAR2), extracellular matrix (e.g., CD209, SERPINA, SERPINB7), and a global down-regulation of genes associated with cell cycle and transcription. A summary of the different pathways involved in Compound 1 sensitivity and genes associated with the pathways is shown in FIG. 25. Several genes in particular were found to be associated with the cytotoxic effects of Compound 1, which demonstrates that activation of these genes can serve as biomarkers for Compound 1 response in DLBCL. This list includes interferon and chemokine related genes (e.g., IL23A, CCL2, IFITM3), cell adhesion genes (e.g., CLDN7), GPCR signaling genes, and apoptosis related genes (e.g., TNF). For example, a comparative representation of the fold changes of exemplary genes, such as IL23A (FIG. 26A), CCL2 (FIG. 26B), and SRGAP1 (FIG. 26C) across different timepoints and different cell line models indicated the increased modulation of these markers in the cell lines where the cytotoxic effects are stronger, were found to be associated with sensitivity to Compound 1 treatment. In addition, comparison of the proteomic results measured at 6 and 18 hours, indicated differential expression of proteins can be associated with sensitivity to Compound 1 treatment (FIG. 27). For example, the protein levels of genes, such as, IKZF3, IKZF1, ZFP91, ETS1, MNT, MEF2B, SNAPC1, KDM4B, TFAP4, UBTF, BAHD1, MBD4, CBX2, TP63, TLE3, FOXP1, ZBTB11, IRF4, MED26, ATF7, ZNF644, KDM5B, USF2, TCF25, KDM4A, L3MBTL2, SNAPC4, KDM5, EBF1, FOXJ2, NFATC1, ZFP36, HDGF, ELF1, PML, MYBL2, SMAD2, CHD2, STAT1, PAX5, STAT2, PYGO2, IRF9, PCGF2, and ATF3 were found to change in response to treatment with Compound 1, with greater changes observed after 18 hours of treatment, relative to 6 hours of treatment.

Taken together, these results demonstrate that up-regulation of genes associated with interferon signaling, cytokine/chemokine signaling, apoptosis, and cell adhesion, and a global down-regulation of genes associated with cell cycle and transcription can serve as biomarkers for sensitivity to Compound 1 treatment. In particular, exemplary genes such as IL23A, CCL2, IFITM3, CLDN7, TNF, and SRGAP1, can serve as biomarkers for sensitivity to Compound 1 treatment.

1. A method of identifying a subject having a hematological cancer who is likely to be responsive to a treatment compound or predicting the responsiveness of a subject having or suspected of having a hematological cancer to a treatment compound, comprising:
(a) obtaining a sample from the subject, wherein the sample optionally comprises a hematological cancer cell;
(b) determining a biomarker level in the sample;
(c) diagnosing the subject as being likely to be responsive to the treatment compound if:
(i) the biomarker level in the sample is detectable; or
(ii) the biomarker level in the sample is an altered level relative to a reference biomarker level;
wherein the treatment compound is a compound of Formula (I): 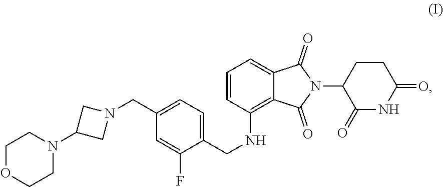 [SMILES: O=C1CCC(N2C(=O)c3cccc(NCc4ccc(CN5CC(N6CCOCC6)C5)cc4F)c3C2=O)C(=O)N1]
or an enantiomer, mixture of enantiomers, tautomer, isotopolog, or pharmaceutically acceptable salt thereof, wherein the compound of Formula (I) optionally comprises
(a) (S)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof;
(b) (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof; or
(c) a mixture of (S)-2-(2,6-Dioxopiperidin-3-yl)-4-((2-fluoro-4-((3-morpholinoazetidin-1-yl)methyl)benzyl)amino)isoindoline-1,3-dione, and (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof;
wherein the biomarker is optionally:
(a) cereblon (CRBN), and wherein the method comprises diagnosing the subject as being likely to be responsive to the treatment compound if CRBN is detectable or higher than a reference level in the sample;
(b) Ikaros, Aiolos, ZFP91, or a combination thereof, and wherein the method comprises diagnosing the subject as being likely to be responsive to the treatment compound if the level of biomarker in the sample is lower than a reference level; or
(c) Ikaros and Aiolos, and wherein the method comprises diagnosing the subject as being likely to be responsive to the treatment compound if the level of both Ikaros and Aiolos are lower than their respective reference levels;
wherein the altered level of the biomarker in the sample is optionally:
(a) an increased biomarker level relative to the reference biomarker level, wherein the increased biomarker level relative to the reference biomarker level is optionally indicative of the efficacy of the treatment compound in treating the hematological cancer in the subject; or
(b) a decreased biomarker level relative to the reference level of the biomarker, wherein the decreased biomarker level relative to the reference biomarker level is optionally indicative of the efficacy of the treatment compound in treating the hematological cancer in the subject;
wherein the reference biomarker level is optionally:
(a) the biomarker level in a reference sample obtained from the subject prior to administering the treatment compound to the subject, and wherein the reference sample is from the same source as the sample;
(b) the biomarker level in a reference sample obtained from a healthy subject not having the hematological cancer, and wherein the reference sample is from the same source as the sample; or
(c) a pre-determined biomarker level;
wherein determining the biomarker level optionally comprises
(a) determining the protein level of the biomarker;
(b) determining the mRNA level of the biomarker; or
(c) determining the cDNA level of the biomarker; and
wherein the hematological cancer optionally:
(a) affects the hematopoietic or lymphoid tissues,
(b) comprises chronic lymphocytic leukemia/small lymphocytic lymphoma (CLL/SLL), wherein the CLL/SLL is optionally relapsed, refractory, or resistant to conventional therapy; and/or
(c) comprises non-Hodgkin's lymphoma, wherein the non-Hodgkin's lymphoma optionally comprises diffuse large B-cell lymphoma (DLBCL), and wherein the DLBCL is optionally relapsed, refractory, or resistant to conventional therapy.

2. A method of selectively treating a hematological cancer in a subject having a hematological cancer, comprising:
(a) obtaining a sample from the subject having a hematological cancer, wherein the sample optionally comprises a hematological cancer cell;
(b) determining a biomarker level in the sample;
(c) diagnosing the subject as being likely to be responsive to a treatment compound if:
(i) the biomarker level in the sample is detectable; or
(ii) the biomarker level is an altered level relative to a reference level of the biomarker; and
(d) administering a therapeutically effective amount of the treatment compound to the subject diagnosed as being likely to be responsive to the treatment compound,
wherein the treatment compound is a compound of Formula (I): 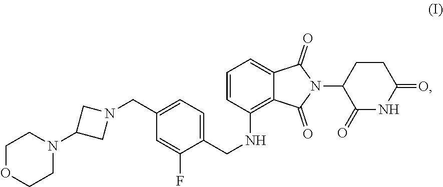 [SMILES: O=C1CCC(N2C(=O)c3cccc(NCc4ccc(CN5CC(N6CCOCC6)C5)cc4F)c3C2=O)C(=O)N1]
or an enantiomer, mixture of enantiomers, tautomer, isotopolog, or pharmaceutically acceptable salt thereof, wherein the compound of Formula (I) optionally comprises
(a) (S)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof;
(b) (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof; or
(c) a mixture of (S)-2-(2,6-Dioxopiperidin-3-yl)-4-((2-fluoro-4-((3-morpholinoazetidin-1-yl)methyl)benzyl)amino)isoindoline-1,3-dione, and (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof;
wherein the biomarker is optionally:
(a) cereblon (CRBN), and wherein the method comprises diagnosing the subject as being likely to be responsive to the treatment compound if CRBN is detectable or higher than a reference level in the sample;
(b) Ikaros, Aiolos, ZFP91, or a combination thereof, and wherein the method comprises diagnosing the subject as being likely to be responsive to the treatment compound if the level of biomarker in the sample is lower than a reference level; or
(c) Ikaros and Aiolos, and wherein the method comprises diagnosing the subject as being likely to be responsive to the treatment compound if the level of both Ikaros and Aiolos are lower than their respective reference levels;
wherein the altered level of the biomarker in the sample is optionally:
(a) an increased biomarker level relative to the reference biomarker level, wherein the increased biomarker level relative to the reference biomarker level is optionally indicative of the efficacy of the treatment compound in treating the hematological cancer in the subject; or
(b) a decreased biomarker level relative to the reference level of the biomarker, wherein the decreased biomarker level relative to the reference biomarker level is optionally indicative of the efficacy of the treatment compound in treating the hematological cancer in the subject;
wherein the reference biomarker level is optionally:
(a) the biomarker level in a reference sample obtained from the subject prior to administering the treatment compound to the subject, and wherein the reference sample is from the same source as the sample;
(b) the biomarker level in a reference sample obtained from a healthy subject not having the hematological cancer, and wherein the reference sample is from the same source as the sample; or
(c) a pre-determined biomarker level;
wherein determining the biomarker level optionally comprises
(a) determining the protein level of the biomarker;
(b) determining the mRNA level of the biomarker; or
(c) determining the cDNA level of the biomarker; and
wherein the hematological cancer optionally:
(a) affects the hematopoietic or lymphoid tissues,
(b) comprises chronic lymphocytic leukemia/small lymphocytic lymphoma (CLL/SLL), wherein the CLL/SLL is optionally relapsed, refractory, or resistant to conventional therapy; and/or
(c) comprises non-Hodgkin's lymphoma, wherein the non-Hodgkin's lymphoma optionally comprises diffuse large B-cell lymphoma (DLBCL), and wherein the DLBCL is optionally relapsed, refractory, or resistant to conventional therapy.

3-5. (canceled)

6. A method of identifying a subject having a hematological cancer who is likely to be responsive to a treatment compound or predicting the responsiveness of a subject having or suspected of having a hematological cancer to a treatment compound, comprising:
(1) (a) obtaining a sample from the subject, wherein the sample optionally comprises a hematological cancer cell;
(b) administering the treatment compound to the sample;
(c) determining a biomarker level in the sample;
(d) diagnosing the subject as being likely to be responsive to the treatment compound if the biomarker level in the sample is an altered level relative to a reference biomarker level; and
optionally further administering a therapeutically effective amount of the treatment compound to the subject diagnosed as being likely to be responsive to the treatment compound; or
(2) (a) administering a treatment compound to a subject;
(b) obtaining a sample from the subject, wherein the sample optionally comprises a hematological cancer cell;
(c) determining a biomarker level in the sample;
(d) diagnosing the subject as being likely to be responsive to the treatment compound if the biomarker level in the sample is an altered level relative to a reference biomarker level, and
optionally further administering a therapeutically effective amount of the treatment compound to the subject diagnosed as being likely to be responsive to the treatment compound;
wherein the treatment compound is a compound of Formula (I): 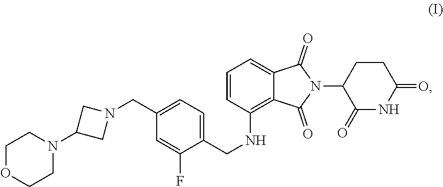 [SMILES: O=C1CCC(N2C(=O)c3cccc(NCc4ccc(CN5CC(N6CCOCC6)C5)cc4F)c3C2=O)C(=O)N1]
or an enantiomer, mixture of enantiomers, tautomer, isotopolog, or pharmaceutically acceptable salt thereof, wherein the compound of Formula (I) optionally comprises
(a) (S)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof;
(b) (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof; or
(c) a mixture of (S)-2-(2,6-Dioxopiperidin-3-yl)-4-((2-fluoro-4-((3-morpholinoazetidin-1-yl)methyl)benzyl)amino)isoindoline-1,3-dione, and (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof,
wherein the altered level of the biomarker in the sample is optionally:
(a) an increased biomarker level relative to the reference biomarker level, wherein the increased biomarker level relative to the reference biomarker level is optionally indicative of the efficacy of the treatment compound in treating the hematological cancer in the subject; or
(b) a decreased biomarker level relative to the reference level of the biomarker, wherein the decreased biomarker level relative to the reference biomarker level is optionally indicative of the efficacy of the treatment compound in treating the hematological cancer in the subject;
wherein the reference biomarker level is optionally:
(a) the biomarker level in a reference sample obtained from the subject prior to administering the treatment compound to the subject, and wherein the reference sample is from the same source as the sample;
(b) the biomarker level in a reference sample obtained from a healthy subject not having the hematological cancer, and wherein the reference sample is from the same source as the sample; or
(c) a pre-determined biomarker level;
wherein determining the biomarker level optionally comprises
(a) determining the protein level of the biomarker;
(b) determining the mRNA level of the biomarker; or
(c) determining the cDNA level of the biomarker; and
wherein the hematological cancer optionally:
(a) affects the hematopoietic or lymphoid tissues,
(b) comprises chronic lymphocytic leukemia/small lymphocytic lymphoma (CLL/SLL), wherein the CLL/SLL is optionally relapsed, refractory, or resistant to conventional therapy; and/or
(c) comprises non-Hodgkin's lymphoma, wherein the non-Hodgkin's lymphoma optionally comprises diffuse large B-cell lymphoma (DLBCL), and wherein the DLBCL is optionally relapsed, refractory, or resistant to conventional therapy.

7. (canceled)

8. A method of monitoring the efficacy of a treatment compound in treating a hematological cancer in a subject, comprising:
(a) administering a treatment compound to a subject;
(b) obtaining a sample from the subject, wherein the sample optionally comprises a hematological cancer cell;
(c) determining a biomarker level in the sample; and
(d) comparing the biomarker level in the sample with a reference biomarker level, wherein an altered biomarker level is indicative of the efficacy of the treatment compound in treating a hematological cancer in the subject,
wherein the method optionally further comprises administering a therapeutically effective amount of the treatment compound to the subject diagnosed as being likely to be responsive to the treatment compound,
wherein the treatment compound is a compound of Formula (I): 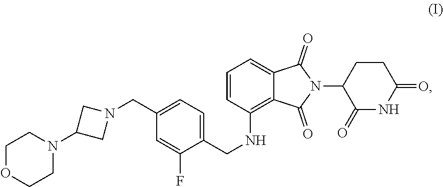 [SMILES: O=C1CCC(N2C(=O)c3cccc(NCc4ccc(CN5CC(N6CCOCC6)C5)cc4F)c3C2=O)C(=O)N1]
or an enantiomer, mixture of enantiomers, tautomer, isotopolog, or pharmaceutically acceptable salt thereof, wherein the compound of Formula (I) optionally comprises
(a) (S)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof;
(b) (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof; or
(c) a mixture of (S)-2-(2,6-Dioxopiperidin-3-yl)-4-((2-fluoro-4-((3-morpholinoazetidin-1-yl)methyl)benzyl)amino)isoindoline-1,3-dione, and (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof; and
wherein the altered level of the biomarker in the sample is optionally:
(a) an increased biomarker level relative to the reference biomarker level,
wherein the increased biomarker level relative to the reference biomarker level is optionally indicative of the efficacy of the treatment compound in treating the hematological cancer in the subject; or
(b) a decreased biomarker level relative to the reference level of the biomarker,
wherein the decreased biomarker level relative to the reference biomarker level is optionally indicative of the efficacy of the treatment compound in treating the hematological cancer in the subject;
wherein the reference biomarker level is optionally:
(a) the biomarker level in a reference sample obtained from the subject prior to administering the treatment compound to the subject, and wherein the reference sample is from the same source as the sample;
(b) the biomarker level in a reference sample obtained from a healthy subject not having the hematological cancer, and wherein the reference sample is from the same source as the sample; or
(c) a pre-determined biomarker level;
wherein determining the biomarker level optionally comprises
(a) determining the protein level of the biomarker;
(b) determining the mRNA level of the biomarker; or
(c) determining the cDNA level of the biomarker; and
wherein the hematological cancer optionally:
(a) affects the hematopoietic or lymphoid tissues,
(b) comprises chronic lymphocytic leukemia/small lymphocytic lymphoma (CLL/SLL), wherein the CLL/SLL is optionally relapsed, refractory, or resistant to conventional therapy; and/or
(c) comprises non-Hodgkin's lymphoma, wherein the non-Hodgkin's lymphoma optionally comprises diffuse large B-cell lymphoma (DLBCL), and wherein the DLBCL is optionally relapsed, refractory, or resistant to conventional therapy.

9. A method of adjusting a dosage amount or frequency for treating a subject having a hematological cancer with a treatment compound comprising:
(a) administering a dosage of a treatment compound to a subject;
(b) obtaining one or more samples from the subject at different time points, wherein the sample optionally comprises a hematological cancer cell;
(c) monitoring a biomarker level in the one or more samples, and
(d) adjusting the dosage for subsequent administration of the treatment compound to the subject based upon an altered level of the biomarker in a reference sample,
wherein the method optionally further comprises administering a therapeutically effective amount of the treatment compound to the subject diagnosed as being likely to be responsive to the treatment compound,
wherein the treatment compound is a compound of Formula (I): 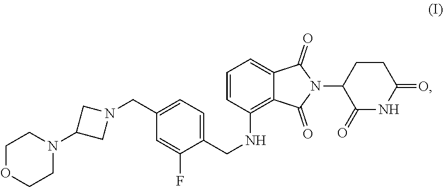 [SMILES: O=C1CCC(N2C(=O)c3cccc(NCc4ccc(CN5CC(N6CCOCC6)C5)cc4F)c3C2=O)C(=O)N1]
or an enantiomer, mixture of enantiomers, tautomer, isotopolog, or pharmaceutically acceptable salt thereof, wherein the compound of Formula (I) optionally comprises
(a) (S)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof;
(b) (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof; or
(c) a mixture of (S)-2-(2,6-Dioxopiperidin-3-yl)-4-((2-fluoro-4-((3-morpholinoazetidin-1-yl)methyl)benzyl)amino)isoindoline-1,3-dione, and (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof;
wherein the altered level of the biomarker in the sample is optionally:
(a) an increased biomarker level relative to the reference biomarker level,
wherein the increased biomarker level relative to the reference biomarker level is optionally indicative of the efficacy of the treatment compound in treating the hematological cancer in the subject; or
(b) a decreased biomarker level relative to the reference level of the biomarker,
wherein the decreased biomarker level relative to the reference biomarker level is optionally indicative of the efficacy of the treatment compound in treating the hematological cancer in the subject;
wherein the reference biomarker level is optionally:
(a) the biomarker level in a reference sample obtained from the subject prior to administering the treatment compound to the subject, and wherein the reference sample is from the same source as the sample;
(b) the biomarker level in a reference sample obtained from a healthy subject not having the hematological cancer, and wherein the reference sample is from the same source as the sample; or
(c) a pre-determined biomarker level;
wherein determining the biomarker level optionally comprises
(a) determining the protein level of the biomarker;
(b) determining the mRNA level of the biomarker; or
(c) determining the cDNA level of the biomarker; and
wherein the hematological cancer optionally:
(a) affects the hematopoietic or lymphoid tissues,
(b) comprises chronic lymphocytic leukemia/small lymphocytic lymphoma (CLL/SLL), wherein the CLL/SLL is optionally relapsed, refractory, or resistant to conventional therapy; and/or
(c) comprises non-Hodgkin's lymphoma, wherein the non-Hodgkin's lymphoma optionally comprises diffuse large B-cell lymphoma (DLBCL), and wherein the DLBCL is optionally relapsed, refractory, or resistant to conventional therapy.

10-17. (canceled)

18. The method of claim 6, wherein the biomarker optionally comprises:
(a) a marker of apoptosis, and wherein the alteration of the biomarker level is indicative of the induction of apoptosis,
wherein the marker of apoptosis is optionally selected from the group consisting of cleaved caspase 3, cleaved caspase 7, cleaved poly (ADP-ribose) polymerase (PARP), BCL2, survivin, phosphatidylserine (PS) and DNA, Bcl-2-like protein 11 (BIM), tumor necrosis factor (TNF), interleukin-10 (IL-10), interleukin-27 (IL27), Annexin-V, 7-amino-actinomycin D (7-AAD), and Deep Red Anthraquinone 7 (DRAQ7), or a combination thereof,
wherein the biomarker is selected from the group consisting of, or a combination thereof
(b) a biomarker selected from the group consisting of IL-8, IL-1a, sPGE2, sTNFα, sIgG, sIL-17A, sIL-17F, sIL-2, sIL-6, collagen-I and -III, PAI-1, CD69, and sIL-10, or a combination thereof;
(c) a biomarker associated with interferon signaling, wherein the biomarker associated with interferon signaling optionally comprises interleukin-6 signal transducer (IL6ST), interferon-induced transmembrane protein 3 (IFITM3), interferon alpha-inducible protein 6 (IFI6), 2′-5′-oligoadenylate synthase 3 (OAS3), interferon α (IFNα), interferon β (IFN β), or a combination thereof;
(d) a biomarker associated with cytokine/chemokine signaling, wherein the biomarker associated with cytokine/chemokine signaling optionally comprises interleukin-23 subunit alpha (IL23A), C—C motif chemokine 1 (CCL1), or a combination thereof;
(e) a biomarker associated with cell adhesion, wherein the biomarker associated with cell adhesion optionally comprises E-selectin (SELE), P-selectin glycoprotein ligand 1 (SELPLG), thromboxane A2 (TXA2), or a combination thereof;
(f) a biomarker associated with cell-cell junction, wherein the biomarker associated with cell-cell junction optionally comprises claudin 7 (CLDN7), claudin 12 (CLDN12), or a combination thereof;
(g) a G-protein coupled receptor, wherein the G-protein coupled receptor optionally comprises free fatty acid receptor 2 (FFAR2);
(h) a biomarker associated with extracellular matrix, wherein the biomarker associated with extracellular matrix optionally comprises CD209, SERPINA, SERPINB7, or a combination thereof;
(i) a biomarker associated with cell cycle;
(j) a biomarker associated with transcription;
(k) one or more proteins selected from the group consisting of Aiolos (IKZF3), Ikaros (IKZF1), E3 ubiquitin-protein ligase ZFP91 (ZFP91), Protein C-ets-1 (ETS1), Max-binding protein MNT (MNT), myocyte-specific enhancer factor 2B (MEF2B), snRNA-activating protein complex subunit 1 (SNAPC1), lysine-specific demethylase 4B (KDM4B), transcription factor AP-4 (TFAP4), nucleolar transcription factor 1 (UBTF), bromo adjacent homology domain-containing 1 protein (BAHD1), methyl-CpG-binding domain protein 4 (MBD4), chromobox protein homolog 2 (CBX2), tumor protein 63 (TP63), transducin-like enhancer protein 3 (TLE3), forkhead box protein P1 (FOXP1), zinc finger and BTB domain-containing protein 11 (ZBTB11), interferon regulatory factor 4 (IRF4), mediator of RNA polymerase II transcription subunit 26 (MED26), cyclic AMP-dependent transcription factor ATF-7 (ATF7), zinc finger protein 644 (ZNF644), lysine-specific demethylase 5B (KDM5B), upstream stimulatory factor 2 (USF2), transcription factor 25 (TCF25), lysine-specific demethylase 4A (KDM4A), lethal (3) malignant brain tumor-like protein 2 (L3MBTL2), nRNA-activating protein complex subunit 4 (SNAPC4), lysine-specific demethylase 5 (KDM5), transcription factor COE1 (EBF1), forkhead box protein J2 (FOXJ2), nuclear factor of activated T-cells, cytoplasmic 1 (NFATC1), mRNA decay activator protein ZFP36 (ZFP36), hepatoma-derived growth factor (HDGF), ETS-related transcription factor Elf-1 (ELF1), promyelocytic leukemia protein (PML), Myb-related protein B MYBL2, mothers against decapentaplegic homolog 2 (SMAD2), chromodomain-helicase-DNA-binding protein 2 (CHD2), signal transducer and activator of transcription 1 (STAT1), Paired box protein Pax-5 (PAX5), Signal transducer and activator of transcription 2 (STAT2), pygopus homolog 2 (PYGO2), interferon regulatory factor 9 (IRF9), polycomb group RING finger protein 2 (PCGF2), and cyclic AMP-dependent transcription factor ATF-3 (ATF3),
(l) one or more genes selected from the group consisting of Interleukin-23 subunit alpha (IL23A), C—C motif chemokine 2 (CCL2), and SLIT-ROBO Rho GTPase-activating protein 1 (SRGAP1);
(m) a CRBN-associated protein or a transcriptional target of a CRBN-associated protein,
wherein the CRBN-associated protein optionally comprises
(i) a CRBN-associated protein selected from the group consisting of IKAROS, AIOLOS, and ZFP91,
(ii) an interferon inducible gene, wherein the interferon inducible gene is optionally selected from the group consisting of interferon regulatory 7 (IRF7), interferon induced protein with tetratricopeptide repeats 3 (IFIT3), DEAD box protein 58 (DDX58), and a combination thereof;
(iii) cyclin dependent kinase inhibitor 1 (p21); and
wherein the transcriptional target of a CRBN-associated protein is optionally selected from the group consisting of BCL6, c-MYC, and IRF4;
(n) a marker of T-cell activation, wherein the marker of T-cell activation optionally comprises a T-cell activation associated cytokine, wherein the T-cell activation associated cytokine optionally comprises interleukin 2 (IL-2);
(o) PD1 and LAG3; and
(p) an effector cytokine or effector chemokine, wherein the effector cytokine or effector chemokine is optionally selected from the group consisting of granulocyte-macrophage colony-stimulating factor (GM-CSF), tumor necrosis factor alpha (TNFα), interferon gamma (IFNγ), and a combination thereof,
wherein the biomarker in the sample is optionally higher than the reference level of the biomarker or optionally lower than the reference level of the biomarker.

19-87. (canceled)

88. The method of claim 1, wherein the biomarker is expressed in a white blood cell, wherein the white blood cell optionally comprises a lymphoid cell, and wherein the lymphoid cell optionally comprises a T-cell.

89-90. (canceled)

91. A method of treating a hematological cancer, comprising:
(a) obtaining a first sample from a subject having a hematological cancer;
(b) determining a biomarker level in the first sample;
(c) administering a therapeutically effective amount of a treatment compound to the subject;
(d) obtaining at least one additional sample from the subject after the treatment; and
(e) determining the biomarker level in the at least one additional sample; and
if the biomarker level in the at least one additional sample is at or near the biomarker level of the first sample, then administering another therapeutically effective amount of the treatment compound to the subject,
wherein the sample optionally comprises a hematological cancer cell;
wherein the treatment compound is a compound of Formula (I): 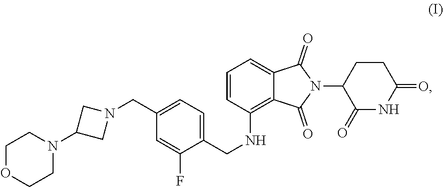 [SMILES: O=C1CCC(N2C(=O)c3cccc(NCc4ccc(CN5CC(N6CCOCC6)C5)cc4F)c3C2=O)C(=O)N1]
or an enantiomer, mixture of enantiomers, tautomer, isotopolog, or pharmaceutically acceptable salt thereof, wherein the compound of Formula (I) optionally comprises
(a) (S)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof;
(b) (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof; or
(c) a mixture of (S)-2-(2,6-Dioxopiperidin-3-yl)-4-((2-fluoro-4-((3-morpholinoazetidin-1-yl)methyl)benzyl)amino)isoindoline-1,3-dione, and (R)-2-(2,6-Dioxopiperidin-3-yl)-4-((2-fluoro-4-((3-morpholinoazetidin-1-yl)methyl)benzyl)amino)isoindoline-1,3-dione, or a tautomer, isotopolog, or pharmaceutically acceptable salt thereof,
wherein the biomarker optionally comprises:
(a) Ikaros, wherein the biomarker is optionally expressed in a white blood cell, wherein the white blood cell optionally comprises a myeloid cell, and wherein the myeloid cell optionally comprises a neutrophil; or
(b) neutrophils having a phenotype of CD11b⁺, CD34⁻, and CD33⁻;
wherein determining the biomarker level optionally comprises:
(a) determining the protein level of the biomarker;
(b) determining the mRNA level of the biomarker; or
(c) determining the cDNA level of the biomarker; and
wherein the hematological cancer optionally:
(a) affects the hematopoietic or lymphoid tissues;
(b) comprises chronic lymphocytic leukemia/small lymphocytic lymphoma (CLL/SLL), wherein the CLL/SLL is optionally relapsed, refractory, or resistant to conventional therapy; and/or
(c) comprises non-Hodgkin's lymphoma, wherein the non-Hodgkin's lymphoma optionally comprises diffuse large B-cell lymphoma (DLBCL), and wherein the DLBCL is optionally relapsed, refractory, or resistant to conventional therapy.

92-99. (canceled)

100. The method of claim 1, further comprising administering a therapeutically effective amount of a second active agent or a support care therapy,
wherein the second active agent optionally comprises:
(a) a second active agent selected from the group consisting of an HDAC inhibitor (e.g., panobinostat, romidepsin, vorinostat, or citarinostat), a BCL2 inhibitor (e.g., venetoclax), a BTK inhibitor (e.g., ibrutinib or acalabrutinib), an mTOR inhibitor (e.g., everolimus), a PI3K inhibitor (e.g., idelalisib), a PKCβ inhibitor (e.g., enzastaurin), a SYK inhibitor (e.g., fostamatinib), a JAK2 inhibitor (e.g., fedratinib, pacritinib, ruxolitinib, baricitinib, gandotinib, lestaurtinib, or momelotinib), an Aurora A kinase inhibitor (e.g., alisertib), an EZH2 inhibitor (e.g., tazemetostat, GSK126, CPI-1205, 3-deazaneplanocin A, EPZ005687, EI1, UNC1999, or sinefungin), a BET inhibitor (e.g., birabresib or 4-[2-(cyclopropylmethoxy)-5-(methanesulfonyl)phenyl]-2-methylisoquinolin-1(2H)-one), a hypomethylating agent (e.g., 5-azacytidine or decitabine), a chemotherapy (e.g., bendamustine, doxorubicin, etoposide, methotrexate, cytarabine, vincristine, ifosfamide, melphalan, dexamethasone, or oxaliplatin), an anti-CD20 monoclonal antibody (e.g., rituximab, or obinutuzumab), an epigenetic compound, or a combination thereof;
(b) rituximab; or
(c) obinutuzumab.

101-113. (canceled)

114. The method of claim 8, wherein the biomarker optionally comprises:
(a) a marker of apoptosis, and wherein the alteration of the biomarker level is indicative of the induction of apoptosis, wherein the marker of apoptosis is optionally selected from the group consisting of cleaved caspase 3, cleaved caspase 7, cleaved poly (ADP-ribose) polymerase (PARP), BCL2, survivin, phosphatidylserine (PS) and DNA, Bcl-2-like protein 11 (BIM), tumor necrosis factor (TNF), interleukin-10 (IL-10), interleukin-27 (IL27), Annexin-V, 7-amino-actinomycin D (7-AAD), and Deep Red Anthraquinone 7 (DRAQ7), or a combination thereof;
(b) a biomarker selected from the group consisting of IL-8, IL-1a, sPGE2, sTNFα, sIgG, sIL-17A, sIL-17F, sIL-2, sIL-6, collagen-I and —III, PAI-1, CD69, and sIL-10, or a combination thereof;
(c) a biomarker associated with interferon signaling, wherein the biomarker associated with interferon signaling optionally comprises interleukin-6 signal transducer (IL6ST), interferon-induced transmembrane protein 3 (IFITM3), interferon alpha-inducible protein 6 (IFI6), 2′-5′-oligoadenylate synthase 3 (OAS3), interferon α (IFNα), interferon (3 (IFN β), or a combination thereof;
(d) a biomarker associated with cytokine/chemokine signaling, wherein the biomarker associated with cytokine/chemokine signaling optionally comprises interleukin-23 subunit alpha (IL23A), C—C motif chemokine 1 (CCL1), or a combination thereof;
(e) a biomarker associated with cell adhesion, wherein the biomarker associated with cell adhesion optionally comprises E-selectin (SELE), P-selectin glycoprotein ligand 1 (SELPLG), thromboxane A2 (TXA2), or a combination thereof;
(f) a biomarker associated with cell-cell junction, wherein the biomarker associated with cell-cell junction optionally comprises claudin 7 (CLDN7), claudin 12 (CLDN12), or a combination thereof;
(g) a G-protein coupled receptor, wherein the G-protein coupled receptor optionally comprises free fatty acid receptor 2 (FFAR2);
(h) a biomarker associated with extracellular matrix, wherein the biomarker associated with extracellular matrix optionally comprises CD209, SERPINA, SERPINB7, or a combination thereof;
(i) a biomarker associated with cell cycle;
(j) a biomarker associated with transcription;
(k) one or more proteins selected from the group consisting of Aiolos (IKZF3), Ikaros (IKZF1), E3 ubiquitin-protein ligase ZFP91 (ZFP91), Protein C-ets-1 (ETS1), Max-binding protein MNT (MNT), myocyte-specific enhancer factor 2B (MEF2B), snRNA-activating protein complex subunit 1 (SNAPC1), lysine-specific demethylase 4B (KDM4B), transcription factor AP-4 (TFAP4), nucleolar transcription factor 1 (UBTF), bromo adjacent homology domain-containing 1 protein (BAHD1), methyl-CpG-binding domain protein 4 (MBD4), chromobox protein homolog 2 (CBX2), tumor protein 63 (TP63), transducin-like enhancer protein 3 (TLE3), forkhead box protein P1 (FOXP1), zinc finger and BTB domain-containing protein 11 (ZBTB11), interferon regulatory factor 4 (IRF4), mediator of RNA polymerase II transcription subunit 26 (MED26), cyclic AMP-dependent transcription factor ATF-7 (ATF7), zinc finger protein 644 (ZNF644), lysine-specific demethylase 5B (KDM5B), upstream stimulatory factor 2 (USF2), transcription factor 25 (TCF25), lysine-specific demethylase 4A (KDM4A), lethal (3) malignant brain tumor-like protein 2 (L3MBTL2), nRNA-activating protein complex subunit 4 (SNAPC4), lysine-specific demethylase 5 (KDM5), transcription factor COE1 (EBF1), forkhead box protein J2 (FOXJ2), nuclear factor of activated T-cells, cytoplasmic 1 (NFATC1), mRNA decay activator protein ZFP36 (ZFP36), hepatoma-derived growth factor (HDGF), ETS-related transcription factor Elf-1 (ELF1), promyelocytic leukemia protein (PML), Myb-related protein B MYBL2, mothers against decapentaplegic homolog 2 (SMAD2), chromodomain-helicase-DNA-binding protein 2 (CHD2), signal transducer and activator of transcription 1 (STAT1), Paired box protein Pax-5 (PAX5), Signal transducer and activator of transcription 2 (STAT2), pygopus homolog 2 (PYGO2), interferon regulatory factor 9 (IRF9), polycomb group RING finger protein 2 (PCGF2), and cyclic AMP-dependent transcription factor ATF-3 (ATF3);
(l) one or more genes selected from the group consisting of Interleukin-23 subunit alpha (IL23A), C—C motif chemokine 2 (CCL2), and SLIT-ROBO Rho GTPase-activating protein 1 (SRGAP1);
(m) a CRBN-associated protein or a transcriptional target of a CRBN-associated protein,
wherein the CRBN-associated protein optionally comprises
(i) a CRBN-associated protein selected from the group consisting of IKAROS, AIOLOS, and ZFP91,
(ii) an interferon inducible gene, wherein the interferon inducible gene is optionally selected from the group consisting of interferon regulatory 7 (IRF7), interferon induced protein with tetratricopeptide repeats 3 (IFIT3), DEAD box protein 58 (DDX58), and a combination thereof;
(iii) cyclin dependent kinase inhibitor 1 (p21); and
wherein the transcriptional target of a CRBN-associated protein is optionally selected from the group consisting of BCL6, c-MYC, and IRF4;
(n) a marker of T-cell activation, wherein the marker of T-cell activation optionally comprises a T-cell activation associated cytokine, wherein the T-cell activation associated cytokine optionally comprises interleukin 2 (IL-2);
(o) PD1 and LAG3; and
(p) an effector cytokine or effector chemokine, wherein the effector cytokine or effector chemokine is optionally selected from the group consisting of granulocyte-macrophage colony-stimulating factor (GM-CSF), tumor necrosis factor alpha (TNFα), interferon gamma (IFNγ), and a combination thereof,
wherein the biomarker in the sample is optionally higher than the reference level of the biomarker or optionally lower than the reference level of the biomarker.

115. The method of claim 9, wherein the biomarker optionally comprises:
(a) a marker of apoptosis, and wherein the alteration of the biomarker level is indicative of the induction of apoptosis, wherein the marker of apoptosis is optionally selected from the group consisting of cleaved caspase 3, cleaved caspase 7, cleaved poly (ADP-ribose) polymerase (PARP), BCL2, survivin, phosphatidylserine (PS) and DNA, Bcl-2-like protein 11 (BIM), tumor necrosis factor (TNF), interleukin-10 (IL-10), interleukin-27 (IL27), Annexin-V, 7-amino-actinomycin D (7-AAD), and Deep Red Anthraquinone 7 (DRAQ7), or a combination thereof;
(b) a biomarker selected from the group consisting of IL-8, IL-1a, sPGE2, sTNFα, sIgG, sIL-17A, sIL-17F, sIL-2, sIL-6, collagen-I and —III, PAI-1, CD69, and sIL-10, or a combination thereof;
(c) a biomarker associated with interferon signaling, wherein the biomarker associated with interferon signaling optionally comprises interleukin-6 signal transducer (IL6ST), interferon-induced transmembrane protein 3 (IFITM3), interferon alpha-inducible protein 6 (IFI6), 2′-5′-oligoadenylate synthase 3 (OAS3), interferon α (IFNα), interferon β (IFN β), or a combination thereof;
(d) a biomarker associated with cytokine/chemokine signaling, wherein the biomarker associated with cytokine/chemokine signaling optionally comprises interleukin-23 subunit alpha (IL23A), C—C motif chemokine 1 (CCL1), or a combination thereof;
(e) a biomarker associated with cell adhesion, wherein the biomarker associated with cell adhesion optionally comprises E-selectin (SELE), P-selectin glycoprotein ligand 1 (SELPLG), thromboxane A2 (TXA2), or a combination thereof;
(f) a biomarker associated with cell-cell junction, wherein the biomarker associated with cell-cell junction optionally comprises claudin 7 (CLDN7), claudin 12 (CLDN12), or a combination thereof;
(g) a G-protein coupled receptor, wherein the G-protein coupled receptor optionally comprises free fatty acid receptor 2 (FFAR2);
(h) a biomarker associated with extracellular matrix, wherein the biomarker associated with extracellular matrix optionally comprises CD209, SERPINA, SERPINB7, or a combination thereof;
(i) a biomarker associated with cell cycle;
(j) a biomarker associated with transcription;
(k) one or more proteins selected from the group consisting of Aiolos (IKZF3), Ikaros (IKZF1), E3 ubiquitin-protein ligase ZFP91 (ZFP91), Protein C-ets-1 (ETS1), Max-binding protein MNT (MNT), myocyte-specific enhancer factor 2B (MEF2B), snRNA-activating protein complex subunit 1 (SNAPC1), lysine-specific demethylase 4B (KDM4B), transcription factor AP-4 (TFAP4), nucleolar transcription factor 1 (UBTF), bromo adjacent homology domain-containing 1 protein (BAHD1), methyl-CpG-binding domain protein 4 (MBD4), chromobox protein homolog 2 (CBX2), tumor protein 63 (TP63), transducin-like enhancer protein 3 (TLE3), forkhead box protein P1 (FOXP1), zinc finger and BTB domain-containing protein 11 (ZBTB11), interferon regulatory factor 4 (IRF4), mediator of RNA polymerase II transcription subunit 26 (MED26), cyclic AMP-dependent transcription factor ATF-7 (ATF7), zinc finger protein 644 (ZNF644), lysine-specific demethylase 5B (KDM5B), upstream stimulatory factor 2 (USF2), transcription factor 25 (TCF25), lysine-specific demethylase 4A (KDM4A), lethal (3) malignant brain tumor-like protein 2 (L3MBTL2), nRNA-activating protein complex subunit 4 (SNAPC4), lysine-specific demethylase 5 (KDM5), transcription factor COE1 (EBF1), forkhead box protein J2 (FOXJ2), nuclear factor of activated T-cells, cytoplasmic 1 (NFATC1), mRNA decay activator protein ZFP36 (ZFP36), hepatoma-derived growth factor (HDGF), ETS-related transcription factor Elf-1 (ELF1), promyelocytic leukemia protein (PML), Myb-related protein B MYBL2, mothers against decapentaplegic homolog 2 (SMAD2), chromodomain-helicase-DNA-binding protein 2 (CHD2), signal transducer and activator of transcription 1 (STAT1), Paired box protein Pax-5 (PAX5), Signal transducer and activator of transcription 2 (STAT2), pygopus homolog 2 (PYGO2), interferon regulatory factor 9 (IRF9), polycomb group RING finger protein 2 (PCGF2), and cyclic AMP-dependent transcription factor ATF-3 (ATF3);
(l) one or more genes selected from the group consisting of Interleukin-23 subunit alpha (IL23A), C—C motif chemokine 2 (CCL2), and SLIT-ROBO Rho GTPase-activating protein 1 (SRGAP1);
(m) a CRBN-associated protein or a transcriptional target of a CRBN-associated protein,
wherein the CRBN-associated protein optionally comprises
(i) a CRBN-associated protein selected from the group consisting of IKAROS, AIOLOS, and ZFP91,
(ii) an interferon inducible gene, wherein the interferon inducible gene is optionally selected from the group consisting of interferon regulatory 7 (IRF7), interferon induced protein with tetratricopeptide repeats 3 (IFIT3), DEAD box protein 58 (DDX58), and a combination thereof;
(iii) cyclin dependent kinase inhibitor 1 (p21); and
wherein the transcriptional target of a CRBN-associated protein is optionally selected from the group consisting of BCL6, c-MYC, and IRF4;
(n) a marker of T-cell activation, wherein the marker of T-cell activation optionally comprises a T-cell activation associated cytokine, wherein the T-cell activation associated cytokine optionally comprises interleukin 2 (IL-2);
(o) PD1 and LAG3; and
(p) an effector cytokine or effector chemokine, wherein the effector cytokine or effector chemokine is optionally selected from the group consisting of granulocyte-macrophage colony-stimulating factor (GM-CSF), tumor necrosis factor alpha (TNFα), interferon gamma (IFNγ), and a combination thereof,
wherein the biomarker in the sample is optionally higher than the reference level of the biomarker or optionally lower than the reference level of the biomarker.

116. The method of claim 2, further comprising administering a therapeutically effective amount of a second active agent or a support care therapy,
wherein the second active agent optionally comprises:
(a) a second active agent selected from the group consisting of an HDAC inhibitor (e.g., panobinostat, romidepsin, vorinostat, or citarinostat), a BCL2 inhibitor (e.g., venetoclax), a BTK inhibitor (e.g., ibrutinib or acalabrutinib), an mTOR inhibitor (e.g., everolimus), a PI3K inhibitor (e.g., idelalisib), a PKCβ inhibitor (e.g., enzastaurin), a SYK inhibitor (e.g., fostamatinib), a JAK2 inhibitor (e.g., fedratinib, pacritinib, ruxolitinib, baricitinib, gandotinib, lestaurtinib, or momelotinib), an Aurora A kinase inhibitor (e.g., alisertib), an EZH2 inhibitor (e.g., tazemetostat, GSK126, CPI-1205, 3-deazaneplanocin A, EPZ005687, EI1, UNC1999, or sinefungin), a BET inhibitor (e.g., birabresib or 4-[2-(cyclopropylmethoxy)-5-(methanesulfonyl)phenyl]-2-methylisoquinolin-1(2H)-one), a hypomethylating agent (e.g., 5-azacytidine or decitabine), a chemotherapy (e.g., bendamustine, doxorubicin, etoposide, methotrexate, cytarabine, vincristine, ifosfamide, melphalan, dexamethasone, or oxaliplatin), an anti-CD20 monoclonal antibody (e.g., rituximab, or obinutuzumab), an epigenetic compound, or a combination thereof;
(b) rituximab; or
(c) obinutuzumab.

117. The method of claim 6, further comprising administering a therapeutically effective amount of a second active agent or a support care therapy,
wherein the second active agent optionally comprises:
(a) a second active agent selected from the group consisting of an HDAC inhibitor (e.g., panobinostat, romidepsin, vorinostat, or citarinostat), a BCL2 inhibitor (e.g., venetoclax), a BTK inhibitor (e.g., ibrutinib or acalabrutinib), an mTOR inhibitor (e.g., everolimus), a PI3K inhibitor (e.g., idelalisib), a PKCβ inhibitor (e.g., enzastaurin), a SYK inhibitor (e.g., fostamatinib), a JAK2 inhibitor (e.g., fedratinib, pacritinib, ruxolitinib, baricitinib, gandotinib, lestaurtinib, or momelotinib), an Aurora A kinase inhibitor (e.g., alisertib), an EZH2 inhibitor (e.g., tazemetostat, GSK126, CPI-1205, 3-deazaneplanocin A, EPZ005687, EI1, UNC1999, or sinefungin), a BET inhibitor (e.g., birabresib or 4-[2-(cyclopropylmethoxy)-5-(methanesulfonyl)phenyl]-2-methylisoquinolin-1(2H)-one), a hypomethylating agent (e.g., 5-azacytidine or decitabine), a chemotherapy (e.g., bendamustine, doxorubicin, etoposide, methotrexate, cytarabine, vincristine, ifosfamide, melphalan, dexamethasone, or oxaliplatin), an anti-CD20 monoclonal antibody (e.g., rituximab, or obinutuzumab), an epigenetic compound, or a combination thereof;
(b) rituximab; or
(c) obinutuzumab.

118. The method of claim 8, further comprising administering a therapeutically effective amount of a second active agent or a support care therapy,
wherein the second active agent optionally comprises:
(a) a second active agent selected from the group consisting of an HDAC inhibitor (e.g., panobinostat, romidepsin, vorinostat, or citarinostat), a BCL2 inhibitor (e.g., venetoclax), a BTK inhibitor (e.g., ibrutinib or acalabrutinib), an mTOR inhibitor (e.g., everolimus), a PI3K inhibitor (e.g., idelalisib), a PKCβ inhibitor (e.g., enzastaurin), a SYK inhibitor (e.g., fostamatinib), a JAK2 inhibitor (e.g., fedratinib, pacritinib, ruxolitinib, baricitinib, gandotinib, lestaurtinib, or momelotinib), an Aurora A kinase inhibitor (e.g., alisertib), an EZH2 inhibitor (e.g., tazemetostat, GSK126, CPI-1205, 3-deazaneplanocin A, EPZ005687, EI1, UNC1999, or sinefungin), a BET inhibitor (e.g., birabresib or 4-[2-(cyclopropylmethoxy)-5-(methanesulfonyl)phenyl]-2-methylisoquinolin-1(2H)-one), a hypomethylating agent (e.g., 5-azacytidine or decitabine), a chemotherapy (e.g., bendamustine, doxorubicin, etoposide, methotrexate, cytarabine, vincristine, ifosfamide, melphalan, dexamethasone, or oxaliplatin), an anti-CD20 monoclonal antibody (e.g., rituximab, or obinutuzumab), an epigenetic compound, or a combination thereof;
(b) rituximab; or
(c) obinutuzumab.

119. The method of claim 9, further comprising administering a therapeutically effective amount of a second active agent or a support care therapy,
wherein the second active agent optionally comprises:
(a) a second active agent selected from the group consisting of an HDAC inhibitor (e.g., panobinostat, romidepsin, vorinostat, or citarinostat), a BCL2 inhibitor (e.g., venetoclax), a BTK inhibitor (e.g., ibrutinib or acalabrutinib), an mTOR inhibitor (e.g., everolimus), a PI3K inhibitor (e.g., idelalisib), a PKCβ inhibitor (e.g., enzastaurin), a SYK inhibitor (e.g., fostamatinib), a JAK2 inhibitor (e.g., fedratinib, pacritinib, ruxolitinib, baricitinib, gandotinib, lestaurtinib, or momelotinib), an Aurora A kinase inhibitor (e.g., alisertib), an EZH2 inhibitor (e.g., tazemetostat, GSK126, CPI-1205, 3-deazaneplanocin A, EPZ005687, EI1, UNC1999, or sinefungin), a BET inhibitor (e.g., birabresib or 4-[2-(cyclopropylmethoxy)-5-(methanesulfonyl)phenyl]-2-methylisoquinolin-1(2H)-one), a hypomethylating agent (e.g., 5-azacytidine or decitabine), a chemotherapy (e.g., bendamustine, doxorubicin, etoposide, methotrexate, cytarabine, vincristine, ifosfamide, melphalan, dexamethasone, or oxaliplatin), an anti-CD20 monoclonal antibody (e.g., rituximab, or obinutuzumab), an epigenetic compound, or a combination thereof;
(b) rituximab; or
(c) obinutuzumab.

120. The method of claim 91, further comprising administering a therapeutically effective amount of a second active agent or a support care therapy,
wherein the second active agent optionally comprises:
(a) a second active agent selected from the group consisting of an HDAC inhibitor (e.g., panobinostat, romidepsin, vorinostat, or citarinostat), a BCL2 inhibitor (e.g., venetoclax), a BTK inhibitor (e.g., ibrutinib or acalabrutinib), an mTOR inhibitor (e.g., everolimus), a PI3K inhibitor (e.g., idelalisib), a PKCβ inhibitor (e.g., enzastaurin), a SYK inhibitor (e.g., fostamatinib), a JAK2 inhibitor (e.g., fedratinib, pacritinib, ruxolitinib, baricitinib, gandotinib, lestaurtinib, or momelotinib), an Aurora A kinase inhibitor (e.g., alisertib), an EZH2 inhibitor (e.g., tazemetostat, GSK126, CPI-1205, 3-deazaneplanocin A, EPZ005687, EI1, UNC1999, or sinefungin), a BET inhibitor (e.g., birabresib or 4-[2-(cyclopropylmethoxy)-5-(methanesulfonyl)phenyl]-2-methylisoquinolin-1(2H)-one), a hypomethylating agent (e.g., 5-azacytidine or decitabine), a chemotherapy (e.g., bendamustine, doxorubicin, etoposide, methotrexate, cytarabine, vincristine, ifosfamide, melphalan, dexamethasone, or oxaliplatin), an anti-CD20 monoclonal antibody (e.g., rituximab, or obinutuzumab), an epigenetic compound, or a combination thereof;
(b) rituximab; or
(c) obinutuzumab.

121. The method of claim 2, wherein the biomarker is expressed in a white blood cell, wherein the white blood cell optionally comprises a lymphoid cell, and wherein the lymphoid cell optionally comprises a T-cell.

122. The method of claim 6, wherein the biomarker is expressed in a white blood cell, wherein the white blood cell optionally comprises a lymphoid cell, and wherein the lymphoid cell optionally comprises a T-cell.

123. The method of claim 8, wherein the biomarker is expressed in a white blood cell, wherein the white blood cell optionally comprises a lymphoid cell, and wherein the lymphoid cell optionally comprises a T-cell.

124. The method of claim 9, wherein the biomarker is expressed in a white blood cell, wherein the white blood cell optionally comprises a lymphoid cell, and wherein the lymphoid cell optionally comprises a T-cell.
